(19) United States
(12) Patent Application Publication
(10) Pub. No.: US 2008/0014247 A1
(43) Pub. Date: Jan. 17, 2008

(54) METAL-CONTAINING FORMULATIONS AND METHODS OF USE

(75) Inventors: Cheng Zai Lu, Newton, MA (US); Paul Schechter, Dover, MA (US); Kailash C. Bhol, Tewksbury, MA (US); Wen Gang Chen, Woburn, MA (US)

Correspondence Address: FISH & RICHARDSON PC, P.O. BOX 1022, MINNEAPOLIS, MN, 55440-1022, US

(73) Assignee: Nucryst Pharmaceuticals

(21) Appl. No.: 11/766,897

(22) Filed: Jun. 22, 2007

Related U.S. Application Data
(60) Provisional application No. 60/841,940, filed on Sep. 1, 2006.
(60) Provisional application No. 60/818,053, filed on Jun. 30, 2006.

(51) Int. Cl. A61K 9/14; A61K 33/38; A61K 9/20; A61P 1/00; A61K 9/48; A61K 9/10

(52) U.S. Cl. 424/436; 424/451; 424/464; 424/489; 424/618

(57) ABSTRACT

Metal-containing materials, as well as their preparation, formulations, and use are disclosed.

### CROSS-REFERENCE TO RELATED APPLICATIONS

This application claims the benefit of the filing date of U.S. Provisional Patent Application Ser. No. 60/818,053, filed Jun. 30, 2006, and U.S. Provisional Patent Application Ser. No. 60/841,940, filed Sep. 1, 2006. The contents of these applications are hereby incorporated by reference.

### TECHNICAL FIELD

The disclosure relates to metal-containing materials, as well as formulations and uses thereof.

### BACKGROUND

It is generally desirable to treat a subject (e.g., a human) that has an undesirable condition. Many different formulations have been developed to treat undesirable conditions. For example, certain forms of silver have been reported to be effective in treating some undesirable skin conditions.

### SUMMARY

The disclosure relates to metal-containing materials, as well as their preparation and use.

In one aspect, the disclosure features a composition including a pharmaceutically acceptable carrier, from 0.1 to five percent by weight of a metal-containing material in the pharmaceutically acceptable carrier, and from two to 20 percent by weight stearic acid in the pharmaceutically acceptable carrier.

In another aspect, the disclosure features a composition including from two to 20 percent by weight stearic acid, from 0.5 to three percent by weight polyoxyl 40 stearate, from three to five percent by weight cetyl alcohol, from two to five percent by weight glycerol monostearate, from one to four percent by weight white petrolatum, from three to five percent by weight isopropyl myristate, from one to ten percent by weight titanium dioxide, and from one to ten percent by weight PEG 400.

In another aspect, the disclosure features a composition including a pharmaceutically acceptable carrier, from 0.1 to five weight percent of a metal-containing material in the pharmaceutically acceptable carrier, from two to 20 percent by weight stearic acid in the pharmaceutically acceptable carrier, from 0.5 to three percent by weight polyoxyl 40 stearate in the pharmaceutically acceptable carrier, from three to five percent by weight cetyl alcohol in the pharmaceutically acceptable carrier, from two to five percent by weight glycerol monostearate in the pharmaceutically acceptable carrier, from one to four percent by weight white petrolatum in the pharmaceutically acceptable carrier, from three to five percent by weight isopropyl myristate in the pharmaceutically acceptable carrier, from one to ten percent by weight titanium dioxide in the pharmaceutically acceptable carrier, and from one to ten weight percent PEG 400 in the pharmaceutically acceptable carrier.

In another aspect, the disclosure features a method of treating a subject including contacting an area of a subject having a condition with a composition. The composition includes a pharmaceutically acceptable carrier, from 0.1 to five percent by weight of a metal-containing material in the pharmaceutically acceptable carrier, and the pharmaceutically acceptable carrier includes from two to 20 percent by weight stearic acid.

In another aspect, the disclosure features a composition including a pharmaceutically acceptable carrier, from 0.01 to five percent by weight of a metal-containing material in the pharmaceutically acceptable carrier, and from 0.1 to ten percent by weight of a stabilizing agent. The composition is a nanodispersion.

In another aspect, the disclosure features a method of treating a subject including contacting an area of a subject having a condition with a composition that is a nanodispersion. The composition includes a pharmaceutically acceptable carrier, from 0.01 to five weight percent of a metal-containing material in the pharmaceutically acceptable carrier; and from 0.1 to ten percent by weight of a stabilizing agent.

In one aspect, the disclosure features a method of inhibiting biofilm proliferation including contacting an area of a subject having a bacterial biofilm with an amount of one or more nanocrystalline metal-containing materials. The bacterial biofilm includes a bacterium that is resistant to an antibiotic.

In one aspect, the disclosure features a method of treating inflammatory bowel disease including contacting a gastrointestinal area of a subject with a therapeutically effective amount of a composition in the form of a nanodispersion. The composition includes a pharmaceutically acceptable carrier, from 0.01 to five weight percent of a metal-containing material in the pharmaceutically acceptable carrier, and from 0.1 to ten percent by weight of a stabilizing agent in the pharmaceutically acceptable carrier.

In another aspect, the disclosure features a method of treating inflammatory bowel disease, including contacting a gastrointestinal area of a subject with a composition at a dosage of from one dose per three hours to once per 24 hours. The composition includes a pharmaceutically acceptable carrier, a metal-containing material, and a stabilizing agent. The composition includes from 20 to 600 mg of the metal-containing material per dose, and the composition is a nanodispersion.

In yet another aspect, the disclosure features a method of treating inflammatory bowel disease, including contacting a gastrointestinal area with a therapeutically effective amount of a composition. The composition includes a freeze-dried powder including a metal-containing material, a stabilizing agent, and a bulking agent.

In another aspect, the disclosure features a method of treating inflammatory bowel disease, including contacting a gastrointestinal area of a subject with a composition at a dosage of from one dose per three hours to once per 24 hours. The composition includes a pharmaceutically acceptable carrier, a metal-containing material, and a stabilizing agent. The composition includes at least 0.4 mg of metal-containing material per kilogram of subject per dose, and the composition is a nanodispersion.

In another aspect, the disclosure features a method of treating inflammatory bowel disease including reconstituting a freeze-dried powder of a metal-containing material to form a composition comprising the metal-containing material; and contacting a gastrointestinal area of a subject with a therapeutically effective amount of the composition.

Embodiments can include one or more of the following features.

In some embodiments, the composition includes from two to ten percent by weight stearic acid in the pharmaceutically acceptable carrier. In some embodiments the composition further comprises at most seven percent by weight (e.g., at most two percent by weight) benzyl alcohol in the pharmaceutically acceptable carrier, from 0.5 to three percent by weight polyoxyl 40 stearate in the pharmaceutically acceptable carrier, from three to five percent by weight cetyl alcohol in the pharmaceutically acceptable carrier, from two to five percent by weight glycerol monostearate in the pharmaceutically acceptable carrier, from one to ten percent by weight white petrolatum in the pharmaceutically acceptable carrier, from three to ten percent by weight isopropyl myristate in the pharmaceutically acceptable carrier, from one to ten percent by weight PEG 400 in the pharmaceutically acceptable carrier, and/or from one to ten percent by weight titanium dioxide in the pharmaceutically acceptable carrier. In some embodiments, the titanium oxide is coated with stearic acid.

In some embodiments, the composition includes from one to two percent by weight polyoxyl 40 stearate, from 3.5 to 4.5 percent by weight cetyl alcohol, from two to four percent by weight glycerol monostearate, from two to ten percent by weight stearic acid, from 1.5 to 3.5 percent by weight white petrolatum, from 3.5 to 4.5 percent by weight isopropyl myristate, from 4.5 to 5.5 percent by weight titanium dioxide, and/or from 5.5 to 6.5 percent by weight PEG 400. In some embodiments, the composition further includes from 0.1 to seven percent by weight benzyl alcohol.

In some embodiments, the composition (e.g., formulation) further includes water. In some embodiments, the composition is non-steroidal, non staining (e.g., non staining to fabric), and/or non nut-allergenic. The composition can be moisturizing and/or can be an emollient. In some embodiments, the composition is an anti-microbial barrier and/or anti-inflammatory. The composition can be spreadable (e.g., have good spreadability). In some embodiments, the composition is in a form selected from the group consisting of creams, foams, gels, lotions, pastes, ointments.

In some embodiments, the stabilizing agent includes docusate sodium, sodium lauryl sulfate, cetrimide, PEG, povidone, propylene glycol, propylene glycol alginate, benzalkonium chloride, poloxamer, polyethylene alkyl ethers, sorbitan esters, xanthan gum, polyvinyl alcohol, lecithin, pectin, polysorbate, and/or sorbitan. The composition comprises from 0.1 to two percent by weight of the stabilizing agent. In some embodiments, prior to incorporation of the stabilizing agent into the composition, the metal-containing material has a surface charge, and when incorporated into the composition, the stabilizing agent attenuates the surface charge. The composition can further include a buffered solution in the pharmaceutically acceptable carrier, for example, a lactate buffer, EDTA buffer, citrate buffer, and/or gluconate buffer. The buffer solution has a pH of at least 3 and/or at most 9 prior to incorporation in the composition. The pharmaceutically acceptable carrier can be in a form of an emulsion, an enema, an aerosol, a wash, a foam, a drop, and/or a spray.

In some embodiments, the metal-containing material includes a nanocrystalline and/or atomically disordered metal-containing material. The metal-containing material can include silver-containing compounds, gold-containing compounds, platinum-containing compounds, and/or palladium-containing compounds. In some embodiments, the metal-containing material includes silver and/or nanocrystalline silver. In some embodiments, the metal-containing material includes atomically disordered, nanocrystalline silver. In some embodiments, the metal-containing material includes silver oxide. The nanocrystalline material can be in the form of particles. The particles can be dispersed in the pharmaceutically acceptable carrier. The dispersed particles can have a maximum dimension of five microns.

In some embodiments, the composition includes particles having a maximum dimension of four hundred nanometers or less (e.g., two hundred nanometers or less) and/or a maximum dimension of at least 10 nanometers. In some embodiments, the composition can further include particles having a dimension of four hundred nanometers or more.

In some embodiments, the nanocrystalline metal-containing material is in a composition that is in the form of a cream. The cream can include from 0.1 to five percent (e.g., from 0.1 to two percent) by weight of nanocrystalline metal-containing material. In some embodiments, the nanocrystalline metal-containing material is contained in a nanodispersion. The nanodispersion can include from 0.00001 to five percent by weight of nanocrystalline metal-containing material. The nanodispersion can further include from 0.1 to ten percent by weight of a stabilizing agent. In some embodiments, the nanocrystalline metal-containing material is in a composition that is in the form of a solution. The solution can include from 0.00001 by weight to five percent by weight of nanocrystalline metal-containing material. In certain embodiments, the one or more nanocrystalline metal-containing materials can include nanocrystalline silver, nanocrystalline silver oxide, silver lactate, silver citrate, and/or ionic silver.

In some embodiments, the composition includes a pharmaceutically acceptable carrier, from 0.01 to five weight percent of a metal-containing material in the pharmaceutically acceptable carrier, and from 0.1 to ten percent by weight of a stabilizing agent in the pharmaceutically acceptable carrier.

In some embodiments, the condition is a skin condition. The condition can be eczema, dermatitis, acne, a microbial condition, a biofilm condition, atopic dermatis, pruritis, itching, ichtyosis, psoriasis, seborrheic dermatis, eczematous dermatitis, ulcer and erosion due to cutaneous trauma (diabetic foot ulcer), cutaneous changes of intrinsic or extrinsic aging, dry skin, and/or epidermolysis bullosa. In some embodiments, the condition is an oral condition, a periodontal condition, an eye condition, a gastrointestinal condition, ulcerative colitis, a respiratory condition, a microbial condition, and/or a biofilm condition.

The composition can be in the form of an intracolonic wash or an enema. In some embodiments, the composition is in the form of an aerosol.

In some embodiments, the area of the subject is the skin, the mucosal membrane, the lungs, and/or the oral cavity.

In some embodiments, the antibiotic is different from the one or more nanocrystalline metal-containing materials. As an example, the antibiotic-resistant bacterium can include methicillin-resistant bacterium, vancomycin-resistant bacterium, benzalkonium chloride-resistant bacterium, gram-positive bacterium, and/or gram-negative bacterium. The metal-containing material can decrease the level of ATP in bacterial cells.

In some embodiments, the amount of the one or more nanocrystalline metal-containing materials is less than or equal to the minimum inhibitory concentration of the one or more nanocrystalline metal-containing materials relative to the bacterium. In some embodiments, the bacterial biofilm is an established bacterial biofilm and the method kills bacteria in the established bacterial biofilm. The method can further include contacting the area at least once per 24 hours, at least once per 12 hours, at least once per 6 hours, at least once per 3 hours, or contacting the area continuously.

In some embodiments, the therapeutically effective amount for treating inflammatory bowel disease is at least 0.4 mg (e.g., at least 4 mg, at least 40 mg) of metal-containing material per kg of the subject. The composition can include at most 600 mg of metal-containing material per dose and/or at least 20 mg of metal-containing material per dose. The method can include contacting the area at a dosage of the composition at least one dose per 24 hours, at least one dose per 12 hours, at least one dose per six hours, and/or at least one dose per three hours.

In some embodiments, the freeze-dried powder is in the form of a suppository, tablet, capsule, and/or pill. In some embodiments, the bulking agent includes mannitol, glycine, gelatin, dextran, glucose, sucrose, and/or lactose. In some embodiments, the freeze-dried powder can include a cryoprotectant, such as glycine, glucose, fructose, sucrose, and/or lactose. In some embodiments, the freeze-dried powder includes a stabilizing agent such as docusate sodium, sodium lauryl sulfate, cetrimide, PEG, povidone, propylene glycol, propylene glycol alginate, benzalkonium chloride, poloxamer, polyethylene alkyl ethers, sorbitan esters, xanthan gum, polyvinyl alcohol, lecithin, pectin, polysorbate, and/or sorbitan.

Embodiments can include one or more of the following advantages.

In some embodiments, the composition (e.g., formulation) can have a cosmetically acceptable appearance, such as a uniform color and texture, and be absent of offensive odors. In some embodiments, the formulation decreases the likelihood of discoloration, mirror formation (e.g., silver-mirror formation), and/or viscosity changes, which can occur over time (e.g., one day, two days, five days, one month, two months, three months, six months, a year) when a metal-containing material is mixed with a number of excipients.

In some embodiments, the formulation (e.g., the pharmaceutically acceptable carrier, the metal-containing formulation) can be easily processed and manufactured. The formulation can be well-tolerated by a subject and/or can be non-irritating. In some embodiments, the formulation (e.g., a cream) is an anti-microbial barrier. The formulation can be anti-inflammatory. The formulation can have enhanced emollient properties, such that the formulation can soften and soothe the skin when applied locally and/or reduce water evaporation from a skin surface. The formulation can be moisturizing, such that the formulation can decrease water evaporation from skin and/or humidify the skin when applied locally. The formulation can be substantially free of steroids. The formulation can be non-allergenic (e.g., non-allergenic to nuts). In some embodiments, the formulation has moisturizing and protecting properties, which can provide therapeutic effects when applied onto an area of a subject. In some embodiments, the formulation has good spreadability, such that the formulation can be spread into a thin layer when topically applied before drying. In some embodiments, when applied to an area of a subject, the formulation can release a steady amount of a therapeutic agent (e.g., a metal-containing material) over a period of time (e.g., at least 30 minutes, at least one hour, at least two hours, at least three hours, at least six hours, at least 12 hours, or at least 24 hours; and/or at most 48 hours, at most 24 hours, at most 12 hours, at most six hours, at most three hours, at most two hours, or at most one hour). In some embodiments, the period of time is from 30 minutes to 48 hours (e.g., from 30 minutes to 24 hours, from one hour to 24 hours, from six hours to 24 hours).

In some embodiments, the formulation including the metal-containing material is non-staining to fabrics and/or is easily removed from fabrics. This can be advantageous in order to avoid permanent staining of clothes, for example, when the formulation is for topical use on the skin.

In such embodiments, a formulation containing the metal-containing material can be prepared with or without additional preservatives. Instead, the metal-containing material can be a preservative. Moreover, in embodiments in which the metal-containing material acts as a preservative, the metal-containing material may be included in a therapeutic formulation containing other therapeutic agents (e.g., the metal-containing material may be included primarily in certain therapeutic compositions to act as a preservative).

In some embodiments, a metal-containing formulation including metal-containing particles of small particle size (e.g., about 400 nm or less, about 300 nm or less, about 200 nm or less, about 150 nm or less, about 100 nm or less, about 50 nm or less, or about 25 nm or less; and/or about 10 nm or more, about 25 nm or more, about 50 nm or more, about 100 nm or more, about 150 nm or more, about 200 nm or more, and/or about 300 nm or more) is more therapeutically effective (e.g., 2× more effect, 5× more effective, 10× more effective, 20× more effective, 50× more effective, 100× more effective) than a metal-containing formulation that does not include metal-containing particles of small particle size, such that a smaller quantity of metal-containing material (e.g., 1/100 of a quantity, 1/50 of a quantity, 1/20 of a quantity, 1/10 of a quantity, ⅕ of a quantity, ½ of a quantity) is needed to achieve the same therapeutic effect when the formulation is administered to a subject, for example, to an open wound, past the skin barrier, and/or to a mucosal or serosal area. A decreased quantity of a metal containing material in a formulation can have decreased toxicological effect on a subject, and can facilitate the administration of a formulation.

In some embodiments, the formulation is used to treat conditions caused by gram-positive, gram-negative, fungal pathogens, and/or antibiotic-resistant bacteria. In some embodiments, the conditions are characterized by the presence of bacterial biofilms, and the concentration of the formulation can be less than or equal to the minimum inhibitory concentration for a bacteria of the biofilm. In some embodiments, the formulation is used to treat inflammatory and/or infectious conditions such as inflammatory bowel disease (IBD), inflammatory skin disease, ear infections, eye conditions (e.g., conjunctivitis), and/or periodontal conditions. In some embodiments, the formulation including a metal-containing material can decrease inflammation in an area of a subject.

Other features and advantages of the methods will be apparent from the description and drawings, and from the claims.

### DESCRIPTION OF DRAWINGS

FIG. 1 is a photograph of an embodiment of formulations including nanocrystalline silver;

FIG. 2 is a schematic view of a deposition system;

FIG. 3 is a graph showing the viscosity of formulations over a three-month period;

FIG. 4 is a graph showing quantitative color measurements for fabric samples prior to staining, at staining, and after washing with Tide™ in hot water and a normal cycle;

FIG. 5 is a graph showing quantitative color measurements for fabric samples prior to staining, at staining, and after washing with Tide with bleach alternative™ in cold water and a normal cycle;

FIG. 6A is a graph showing the decrease in bacterial ATP after treatment of Ps. aeruginosa with nanocrystalline silver;

FIG. 6B is a graph showing bacterial ATP content after treatment of Ps. aeruginosa with ciprofloxacin;

FIG. 7 are graphs showing hydrogen peroxide generation (left) and superoxide generation (right) by nanocrystalline silver (-▪-) determined in deionized water, positive controls (-▴-) were reagent-grade hydrogen peroxide and enzymatically generated superoxide, respectively;

FIG. 8 are photographs showing two-day old Pseudomonas biofilms without (left) and with (right) 2-hour treatment with nanocrystalline silver;

FIG. 9 are photographs showing Pseudomonas aeruginosa biofilms grown in presence of 0, 1, 5, or 10 μg/ml of nanocrystalline silver;

FIG. 10 is a chart showing mean body weight change of rats with ulcerative colitis following intracolonic treatment (n=10-20) starting on day 2;

FIG. 11 is a chart showing mean body weight change of rats with ulcerative colitis following oral treatment (n=18-20) starting on day 2;

FIG. 12 is a chart showing the effects of intracolonic treatment of nanocrystalline silver and sulfasalazine on DNBS-induced colitis after five days of treatment, the data represent total IBD score (Mean±SE, n=10-20);

FIG. 13 is a chart showing shows the effects of oral treatment of nanocrystalline silver and sulfasalazine on DNBS-induced colitis after five days of treatment, the data represent total IBD score (Mean±SE, n=18-20);

FIG. 14 is a representative photograph showing of a colon before (left) and after (right) treatment with nanocrystalline silver;

FIGS. 15A-15D are representative photographs showing sections of hematoxylin and eosin stained colon tissues after oral treatment with nanocrystalline silver. 15A was treated with 40 mg/kg nanocrystalline silver, 15B was treated with 100 mg/kg sulfasalazine, 15C was treated with placebo, and 15D was untreated;

FIG. 16 is a chart showing histopathological inflammation scores of colon of DNBS-induced colitis after five days oral treatment with nanocrystalline silver and sulfasalazine (Mean±SE, n=10);

FIGS. 17A-17C are charts showing semi-quantitative assessment of immuno-histochemical staining of TNF-α (A), IL-12 (B), IL-1β (C) of DNBS-induced colitis after five days of oral treatment with nanocrystalline silver and sulfasalazine (Mean±SE, n=10);

FIG. 18 is a representative picture of a gelatin zymography using colonic homogenates of DNBS-induced colitis after oral treatment with nanocrystalline silver and sulfasalazine. Lane 1, standard; 2, normal colon; 3, sulfasalazine 100 mg/kg; 4, nanocrystalline silver 40 mg/kg; 5, nanocrystalline silver 4 mg/kg; 6, nanocrystalline silver 0.4 mg/kg; 7, placebo; and 8, untreated;

FIG. 19 is a chart showing a semi-quantitative assessment of immuno-histochemical staining of MMP-9.

FIG. 20 is a dose response graph showing the effects of different concentrations of nanocrystalline silver-containing cream on erythema scores (Mean±S.E) in the guinea pig model of allergic contact dermatitis. (n=8-18).

FIG. 21 is a dose response graph showing the effects of different concentrations of nanocrystalline silver-containing cream on edema scores (Mean±S.E) in the guinea pig model of allergic contact dermatitis (n=8-18).

FIG. 22 is a graph showing the concentration response of the effects of nanocrystalline silver-containing cream on erythema and edema in the guinea pig model of allergic contact dermatitis.

FIG. 23 is a chart showing the histopathological inflammation (means +SE; n=8-18) after five days of treatment with different concentrations of nanocrystalline silver-containing cream.

FIG. 24 is a graph showing concentration response of the effects of nanocrystalline silver-containing cream on histopathological inflammation score in the guinea pig model of allergic contact dermatitis.

FIG. 25 is a chart showing the effects of silver oxide on the total IBD score in a rat model of DNBS-induced colitis.

FIG. 26 is a chart showing the effects of silver oxide nanodispersion on the total IBD score in a rat model of DNBS-induced colitis.

### DETAILED DESCRIPTION

Certain metal-containing materials (e.g., antimicrobial, anti-inflammatory, atomically disordered, nanocrystalline silver-containing materials) can be used to treat a subject with a condition by contacting an area of the subject having the condition with the metal-containing material. As explained below, the metal-containing material can be in any of a variety of forms when delivered to a subject, and the metal-containing material can be delivered to a subject in a variety of ways. As also explained below, the metal-containing material can be used to treat various subjects, conditions, and condition locations.

In general, the metal-containing material can be in any desired form or composition (e.g., formulation). The material can be, for example, a coating on a substrate (e.g., in the form of a dressing, a coated medical implant), a free standing powder, or disposed within a pharmaceutically acceptable carrier. The metal-containing material can be a component of a formulation such as a cream, a nanodispersion, a solution, a powder (e.g., a free standing powder, a freeze-dried powder), a foam, a gel, a lotion, a paste, an ointment, a spray, a drop, or a suppository, each having a specific formulation. In general, depending on the formulation, the metal-containing material can be used to treat a variety of conditions. For example, a therapeutically effective amount of a cream, nanodispersion, solution, foam, gel, lotion, paste, ointment, spray, or drop, or powder including the metal-containing material can be used to treat skin conditions by directly administering to the affected areas in a subject. Treatment can continue until the condition ameliorates or disappears. A therapeutically effective amount refers to an amount of active compound or pharmaceutical agent that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder (i.e., arresting further development of the pathology and/or symptomatology) such as lowering the bacterial load in the case of a bacterial infection or lowering viral load in the case of a viral infection and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as reducing infection-related tissue damage in the case of a bacterial infection or reducing infection-related cell damage in the case of a viral infection.

Creams

In some embodiments, the formulation is a cream that has a cosmetically acceptable appearance, such as a uniform color and texture, and be absent of offensive odors. The metal-containing material can be dispersed (e.g., uniformly distributed) within a cream. The metal-containing material can be in the form of particles having a maximum dimension of at most five microns (e.g., at most four microns, at most three microns, at most two microns, at most one micron, at most 500 nm, at most 400 nm, at most 300 nm, at most 200 nm, or at most 100 nm). In some embodiments, the particles are agglomerated, and can form clusters of agglomerated particles having a maximum dimension of at most 25 microns (e.g., at most 20 microns, at most 15 microns, or at most 10 microns).

The cream can include components such as: water, cetearyl alcohol, glycerol monostearate, stearic acid, light mineral oil, isopropyl myristate, polyoxyl 40 stearate, propylparaben, methylparaben, xanthan gum (e.g., Xantural), white petrolatum, polyethylene glycol (e.g., PEG 400, PEG 300), titanium dioxide, propylene glycol, diethylene glycol monoethyl ether (Transcutol), cetyl alcohol, benzyl alcohol, hexylene glycol, EDTA, (hydroxypropyl)methylcellulose (HPMC), sodium benzoate, hydroxypropylcellulose (HPC), methyl cellulose (e.g., methyl cellulose A4M), sodium carboxymethylcellulose, sodium parabens, crosslinked polyacrylate polymer (e.g., Carbopol), and/or carrageenan.

In some embodiments, the cream includes at least 0.1 percent (e.g., at least 0.2 percent, at least 0.3 percent, at least 0.4 percent, at least 0.5 percent, at least 0.6 percent, at least 0.8 percent, at least one percent, at least 1.5 percent, at least two percent, at least three percent, at least four percent) and/or at most five percent (at most four percent, at most three percent, at most two percent, at most 1.5 percent, at most one percent, at most 0.8 percent, at most 0.6 percent, at most 0.5 percent, at most 0.4 percent, at most 0.3 percent, or at most 0.2 percent) by weight of a metal-containing material. In some embodiments, the cream includes from 0.1 to five (e.g., from 0.1 to two, from 0.1 to one, from 0.1 to 0.5, from 0.2 to four, from 0.4 to three, from 1 to three, from two to three) percent by weight of a metal-containing material.

In some embodiments, the cream includes at least one percent (e.g., at least 1.5 percent at least two percent, at least three percent, at least 3.5 percent, at least four percent, at least five percent, at least six percent, at least seven percent, at least eight percent, at least nine percent) and/or at most ten percent (e.g., at most nine percent, at most eight percent, at most seven percent, at most six percent, at most five percent, at most four percent, at most 3.5 percent, at most three percent, at most two percent, or at most 1.5 percent) by weight of white petrolatum. In some embodiments, the cream includes from one to ten (e.g., from one to eight, from 1.5 to 3.5, from two to seven, from three to seven, from four to seven, from four to six) percent by weight of white petrolatum. White petrolatum is an emollient, and can moisturize an area of the skin by decreasing evaporation from the skin. In some embodiments, white petrolatum is a microbial barrier.

In some embodiments, the cream includes at least one percent (e.g., at least two percent, at least three percent, at least four percent, at least five percent, at least six percent, at least seven percent, at least eight percent, at least nine percent) and/or at most ten percent (e.g., at most nine percent, at most eight percent, at most seven percent, at most six percent, at most five percent, at most four percent, at most three percent, or at most two percent) by weight of isopropyl myristate. In some embodiments, the cream includes from one to ten (e.g., from two to nine, from three to eight, from three to ten, from 3.5 to 4.5, from four to seven, from four to six) percent by weight of isopropyl myristate. Isopropyl myristate can be a moisturizing agent and an emollient, and can be unreactive with the metal-containing material. In some embodiments, isopropyl myristate is a vehicle for the metal-containing material and can enhance the absorption of the metal-containing material through the skin.

In some embodiments, the cream includes at least 0.5 percent (e.g., at least one percent, at least two percent, at least three percent, at least 3.5 percent, or at least four percent) and/or at most five percent (e.g., at most four percent, at most 3.5 percent, at most three percent, at most two percent, or at most one percent) by weight of polyoxyl 40 stearate. In some embodiments, the cream includes from 0.5 to five (e.g., from one to five, from one to four, from one to three, from one to two, from two to five, from two to four) percent by weight of polyoxyl 40 stearate. Polyoxyl 40 stearate is a nonionic surface-active agent, and can be an emulsifying agent in a cream.

In some embodiments, the cream includes at least two percent (e.g., at least three percent, at least four percent, at least five percent, at least six percent, at least seven percent, at least eight percent, or at least nine percent) and/or at most ten percent (e.g., at most nine percent, at most eight percent, at most seven percent, at most six percent, at most five percent, at most four percent, or at most three percent) by weight of cetearyl alcohol. In some embodiments, the cream includes from two to ten (e.g., from two to nine, from three to eight, from three to ten, from four to seven, from four to six) percent by weight of cetearyl alcohol. Cetearyl alcohol can form an occlusive film and decrease the likelihood of skin moisture evaporation.

In some embodiments, the cream includes at least one percent (e.g., at least two percent, at least three percent, at least 3.5 percent, or at least four percent) and/or at most five percent (e.g., at most four percent, at most 3.5 percent, at most three percent, or at most two percent) by weight of cetyl alcohol. In some embodiments, the cream includes from one to five (e.g., from one to four, from one to three, from two to five, from two to four, from three to five, from 3.5 to 4.5, from four to five) percent by weight of cetyl alcohol. In some embodiments, cetyl alcohol is an emollient, a thickening agent, and/or can lighten the color of the cream (e.g., a cream including a metal-containing material).

In some embodiments, the cream includes at least one percent (e.g., at least two percent, at least three percent, at least 3.5 percent, or at least four percent) and/or at most five percent (e.g., at most four percent, at most 3.5 percent, at most three percent, or at most two percent) by weight of glycerol monostearate. In some embodiments, the cream includes from one to five (e.g., from one to four, from one to three, from two to five, from two to four, from three to five, from four to five) percent by weight of glycerol monostearate. Glycerol monostearate can provide moisturizing properties and/or can thicken the cream.

In some embodiments, the cream includes at least one percent (e.g., at least two percent, at least three percent, at least 3.5 percent, at least four percent, at least five percent, at least six percent, at least seven percent, at least eight percent, at least nine percent, at least ten percent, at least 12 percent, at least 15 percent, or at least 17 percent) and/or at most 20 percent (e.g., at most 17 percent, at most 15 percent, at most 12 percent, at most ten percent, at most nine percent, at most eight percent, at most eight percent, at most seven percent, at most six percent, at most five percent, at most four percent, at most 3.5 percent, at most three percent, or at most two percent) by weight of stearic acid. In some embodiments, the cream can include from one to 20 (e.g., from one to 15, from one to 10, from two to ten, from two to eight, from two to seven, from three to eight, from three to six, from four to six) percent by weight of stearic acid. Addition of stearic acid can result in a lighter-colored cream, and can decrease the likelihood of discoloration. In some embodiments, stearic acid can stabilize the cream and can help maintain the cream in a similar color and texture for a period of time (e.g., at least one month, at least two months, at least three months, at least six months, or at least a year) after cream formation. In some embodiments, stearic acid is an emollient and can provide moisturizing properties to skin.

In some embodiments, the cream includes at least one percent (e.g., at least two percent, at least three percent, at least four percent, at least five percent, at least six percent, at least seven percent, at least eight percent, or at least nine percent) and/or at most ten percent (e.g., at most nine percent, at most eight percent, at most seven percent, at most six percent, at most five percent, at most four percent, at most three percent, or at most two percent) by weight of a poly(ethylene glycol). In some embodiments, the cream includes from one to ten (e.g., from two to nine, from three to eight, from three to ten, from four to seven, from four to six, from five to seven, from 5.5 to 6.5) percent by weight of polyethylene glycol. An example of a polyethylene glycol is PEG 400. PEG 400 can be compatible with a metal-containing material and enhance the texture of the cream to produce a smooth feeling during application. In some embodiments, PEG 400 can stabilize the cream. In some embodiments, PEG 400 can slow the drying process of the formulation and moisturize a skin area to which the cream has been applied.

In some embodiments, the cream includes at least 0.1 percent (e.g., at least 0.2 percent, at least 0.3 percent, at least 0.4 percent, at least 0.5 percent, at least 0.6 percent, at least 0.7 percent, at least 0.8 percent, at least one percent, at least two percent, at least three percent, or at least four percent) and/or at most five percent (e.g., at most four percent, at most three percent, at most two percent, at most one percent, at most 0.8 percent, at most 0.7 percent, at most 0.6 percent, at most 0.5 percent, at most 0.4 percent, at most 0.3 percent, or at most 0.2 percent) by weight of benzyl alcohol. In some embodiments, the cream includes from 0.1 to seven (e.g., from 0.1 to five, from 0.1 to two, from 0.1 to one, from 0.1 to 0.5, from 0.2 to four, from 0.4 to three, from one to two, from 1 to three, from two to three) percent by weight of benzyl alcohol. In some embodiments, benzyl alcohol is a preservative and can decrease the likelihood of microbial proliferation in the cream. In some embodiments, benzyl alcohol is absent when the cream includes a metal-containing material.

In some embodiments, the cream includes at least one percent (e.g., at least two percent, at least three percent, at least four percent, at least five percent, at least six percent, at least seven percent, at least eight percent, or at least nine percent) and/or at most 10 percent (e.g., at most nine percent, at most eight percent, at most seven percent, at most six percent, at most five percent, at most four percent, at most three percent, at most two percent, or at most one percent) by weight of titanium dioxide. In some embodiments, the cream includes from one to ten (e.g., from two to nine, from three to eight, from three to ten, from four to seven, from four to six, from five to seven, from 5.5 to 6.5) percent by weight of titanium dioxide. Titanium dioxide can lighten the color of the cream, for example, to provide a more aesthetically pleasing color. In some embodiments, titanium dioxide is coated with stearic acid prior to addition to a cream. In certain embodiments, titanium dioxide is coated with stearic acid in situ during formation of the cream. In some embodiments, titanium dioxide is not coated with stearic acid prior to addition to a formulation, or during formation of the cream.

In some embodiments, the cream includes at least two percent (e.g., at least three percent, at least four percent, at least five percent, at least six percent, at least seven percent, at least eight percent, or at least nine percent) and/or at most ten percent (e.g., at most nine percent, at most eight percent, at most seven percent, at most six percent, at most five percent, at most four percent, or at most three percent) by weight of light mineral oil. In some embodiments, the cream includes from two to ten (e.g., from two to nine, from three to eight, from three to ten, from four to seven, from four to six, from five to seven) percent by weight of light mineral oil. Light mineral oil is an emollient and can moisturize the skin to which the cream is applied.

In certain embodiments, the cream can include at least 0.01 percent (e.g., at least 0.02 percent, at least 0.03 percent, at least 0.05 percent, at least 0.1 percent, at least 0.2 percent, at least 0.3 percent, at least 0.4 percent) and/or at most 0.5 percent (e.g., at most 0.4 percent, at most 0.3 percent, at most 0.2 percent, at most 0.1 percent, at most 0.05 percent, at most 0.03 percent) by weight of propyl paraben. In some embodiments, the cream can include from 0.01 to 0.5 (e.g., from 0.01 to 0.3, from 0.02 to 0.4, from 0.01 to 0.05, from 0.1 to 0.3) percent by weight of propyl paraben. Propyl paraben can be a preservative and decrease the likelihood of microbial proliferation in the cream. In some embodiments, propyl paraben is absent when the cream contains a metal-containing material.

In certain embodiments, the cream can include at least 0.05 percent (e.g., at most 0.1 percent, at most 0.15 percent, at most 0.2 percent, at most 0.3 percent, or at most 0.4 percent) and/or at most 0.5 percent (e.g., less than 0.4 percent, at most 0.3 percent, less than 0.2 percent, at most 0.15 percent, or at most 0.1 percent) by weight of methyl paraben. In some embodiments, the cream includes from 0.05 to 0.5 (e.g., from 0.1 to 0.4, from 0.2 to 0.3) percent by weight of methyl paraben. Methyl paraben can be a preservative and decrease the likelihood of microbial proliferation in the cream. In some embodiments, methyl paraben is absent when the cream contains a metal-containing material.

In certain embodiments, the cream can include at least 0.02 percent (e.g., at least 0.05 percent, at least 0.1 percent, at least 0.2 percent, at least 0.3 percent, or at least 0.4 percent) and/or at most 0.5 percent (e.g., at most 0.4 percent, at most 0.3 percent, at most 0.2 percent, at most 0.1 percent, or at most 0.05 percent) by weight of xanthan gum. In some embodiments, the cream includes from 0.02 to 0.5 (e.g., from 0.1 to 0.4, from 0.2 to 0.3) percent by weight of xanthan gum. Xanthan gum can help thicken a formulation and help suspend the components of the cream to form a homogeneous mixture. In some embodiments, xantham gum is absent when the cream contains a metal-containing material.

In some embodiments, the cream can include at least 20 percent (e.g., at least 30 percent, at least 50 percent, at least 70 percent, at least 80 percent, or at least 90 percent) and/or at most 99 percent (e.g., at most 90 percent, at most 80 percent, at most 70 percent, at most 50 percent, at most 30 percent) by weight water.

In some embodiments, the cream can include at least zero percent (e.g., at least 0.1 percent, at least 0.3 percent, at least 0.5 percent, at least 0.7 percent, at least 0.9 percent, at least one percent, at least 1.2 percent, at least 1.4 percent, at least 1.6 percent, at least 1.8 percent) and/or at most two percent (e.g., at most 1.8 percent, at most 1.6 percent, at most 1.4 percent, at most 1.2 percent, at most 0.9 percent, at most 0.7 percent, at most 0.5 percent, at most 0.3 percent, at most 0.1 percent) by weight of iron oxide. In some embodiments, the cream includes from 0 to two (e.g., from 0.5 to one, from 0.3 to 0.5, from 0.3 to one) percent by weight iron oxide. Iron oxide can be added for color matching between creams having different concentrations of a metal-containing material.

Nanodispersions

In some embodiments, the material can be in the form of a nanodispersion. A nanodispersion refers to a suspension of one or more metal-containing materials including small particles having a maximum dimension of about 400 nm or less (e.g., about 300 nm or less, about 200 nm or less, about 150 nm or less, about 100 nm or less, about 50 nm or less, about 25 nm or less) and/or at least 10 nm (e.g., at least 25 nm, at least 50 nm, at least 100 nm, at least 150 nm, at least 200 nm, at least 300 nm). In some embodiments, the particles have a maximum dimension of from 10 to 400 (e.g., from 10 to 200, from 10 to 75, from 10 to 50, from 10 to 40) nanometers. In some embodiments, in addition to small particles, the nanodispersion can further include large particles having a maximum dimension of 400 nm or more (e.g., 300 nm or more, 200 nm or more, 150 nm or more, about 100 nm or more, about 50 nm or more, about 25 nm or more). The small particles can be released from micron-sized particles and/or from the large particles, for example, by ultrasonication. A nanodispersion can a be a stable or unstable system of particles evenly distributed in a solvent. The nanodispersion can be substantially more therapeutically effective (e.g., 2× more effect, 5× more effective, 10× more effective, 20× more effective, 50× more effective, 100× more effective) than a suspension of metal-containing materials that does not contain small particles, such that a smaller quantity of metal-containing material (e.g., 1/100 of a quantity, 1/50 of a quantity, 1/20 of a quantity, 1/10 of a quantity, ⅕ of a quantity, ½ of a quantity) is needed in a nanodispersion to achieve the same therapeutic effect as a suspension that does not contain small particles. A decreased quantity of a metal-containing material in a formulation can have decreased toxicological effect on a subject, and can facilitate the administration of a formulation.

Without wishing to be bound by theory, it is believed that the particles within a nanodispersion can contain a mixture of metal-containing material in various proportions. For example, in some embodiments, the metal-containing material in a nanodispersion includes less than 70% (e.g., less than 60%, less than 50%, less than 40%, less than 30%) and/or more than 25% (e.g., more than 30%, more than 40%, more than 50%, more than 60%) by weight Ag(0); less than 70% (e.g., less than 65%, less than 55%, less than 45%, less than 35%) and/or more than 30% (e.g., more than 35%, e.g., more than 45%, more than 55%, more than 65%) by weight Ag₂O; and/or less than 10% (e.g., less than 7%, less than 5%, less than 3%) and/or more than 1% (e.g., more than 3%, more than 5%, more than 7%) Ag₂CO₃.

The nanodispersion can be formed, for example, by dispersing a free standing powder of the material in a solution and sonicating the mixture. In some embodiments, a container (e.g., a tea bag-type container) with the free standing powder within it can be immersed in the water or solvent to disperse the free standing powder, the mixture can then be sonicated using an ultrasonicator (e.g., a probe sonicator such as Hielscher UP400S and/or Sonifier Model #250). In some embodiments, a substrate (e.g., in the form of a strip or a bandage) carrying the material can be immersed in the solvent to disperse the metal-containing material. The solvent containing the substrate can be shaken in a shaking incubator (e.g., at 180 RPM and 37° C. for 30 minutes) and/or stirred, and the mixture can then be sonicated. In some embodiments, formation of the nanodispersion further includes separating a supernatant nanodispersion from a precipitate, for example, by decantation and/or by filtration.

The nanodispersion solvent can be an aqueous or an organic solvent. For example, the solvent can be an alcohol (e.g., propanol, ethanol), an organic solvent (e.g., DMSO, azone), or water. The aqueous solvent can be a solution or a buffer, such as a lactate buffer, an EDTA buffer, a citrate buffer, a glycolate buffer, or a gluconate buffer. The buffer can have a pH at least 3 (e.g., at least 4, at least 5, at least 6, at least 7, or at least 8) and/or at most 9 (e.g., at most 8, at most 7, at most 6, at most 5, or at most 4). In some embodiments, the buffer has a pH of from 3 to 9 (e.g., from 4 to 8, from 3 to 7, from 4 to 6, from 5 to 7). The buffer concentration can be at least 0.05 M (e.g., at least 0.1 M, at least 0.2 M, at least 0.3 M, at least 0.4 M) and/or at most one M (e.g., at most 0.5M, at most 0.4 M, at most 0.3 M, at most 0.2 M, at most 0.1 M). In some embodiments, the buffer concentration can be from 0.05 to one M (e.g., from 0.1 to one M, from 0.2 to 0.5 M, from 0.3 to 0.5 M).

The nanodispersion can include a stabilizing agent, such as surfactant and/or an emulsifier. A stabilizing agent stabilizes nanodispersions by decreasing the likelihood of agglomeration of individual particles. Examples of stabilizing agents include surfactants and/or emulsifiers, such as docusate sodium, sodium lauryl sulfate, cetrimide, PEG, povidone, propylene glycol, propylene glycol alginate, benzalkonium chloride, poloxamer, polyethylene alkyl ethers, sorbitan esters, xanthan gum, polysorbate (e.g., Tween 80), lecithin, pectin, polysorbate, sorbitan (e.g., SPAN) and/or polyvinyl alcohol (PVA). In some embodiments, a stabilizing agent helps suspend the metal-containing material and provides a homogeneous nanodispersion. In some embodiments, a stabilizing agent lowers the surface charge of the particles and decreases the attraction between the particles. In some embodiments, a stabilizing agent (e.g., PVA) acts as a physical barriers between particles to decrease contact between the particles. In other embodiments, stabilizing agents (e.g., lecithin) change the charge on the particles to increase particle-particle repulsion. The surface charge on the particles is assessed by measuring the zeta potential using a Zetasizer nano-ZS instrument (Malvern Instruments Ltd).

The nanodispersion can include one or more surfactants/emulsifiers at a concentration of at least 0.1 percent by weight (e.g., at least 0.5 percent by weight, at least one percent by weight, at least two percent by weight, at least three percent by weight, at least four percent by weight, at least five percent by weight, at least six percent by weight, at least seven percent by weight, at least eight percent by weight, or at least nine percent by weight) and/or at most ten percent by weight (e.g., at most nine percent by weight, at most eight percent by weight, at most seven percent by weight, at most six percent by weight, at most five percent by weight, at most four percent by weight, at most three percent by weight, at most two percent by weight, at most one percent by weight, or at most 0.5 percent by weight). In some embodiments, the nanodispersion includes from 0.01 to ten (e.g., from 0.01 to eight, from 0.5 to eight, from 0.5 to six, from 0.5 to four, from 0.5 to two, from one to eight, from one to six, from one to two, from two to eight, from two to six, from two to three, from four to eight, from four to six) percent by weight of one or more surfactants/emulsifiers.

The nanodispersion can include one or more metal-containing materials at a concentration of at least 0.00001 percent by weight (e.g., greater than 0.0001 percent by weight, at least 0.001 percent by weight, at least 0.01 percent by weight, at least 0.1 percent by weight, at least one percent by weight, at least two percent, at least three percent, at least four percent) and/or at most five percent by weight (e.g., at most four percent, at most three percent, at most two percent, at most one percent by weight, at most 0.1 percent by weight, at most 0.01 percent by weight, at most 0.001 percent by weight, at most 0.0001 percent by weight). In some embodiments, the nanodispersion includes from 0.00001 to five (e.g., from 0.0001 to five, from 0.001 to five, from 0.01 to five, from 0.1 to five, from one to five, from 0.0001 to three, from 0.001 to three, from 0.01 to three, from 0.1 to three, from one to three, from 0.0001 to one, from 0.001 to one, from 0.01 to one, from 0.1 to one, from 0.1 to 0.5) percent by weight of one or more metal-containing materials. In certain embodiments, the nanodispersion includes the metal-containing material at a concentration of at least 1 μg of metal-containing material per one ml nanodispersion (e.g., at least 5 μg/ml, at least 10 μg/ml, at least 20 μg/ml, at least 30 μg/ml, at least 40 μg/ml, at least 50 μg/ml, at least 75 μg/ml) and/or at most 100 μg/ml (e.g., at most 75 μg/ml, at most 50 μg/ml, at most 40 μg/ml, at most 30 μg/ml, at most 20 μg/ml, at most 10 μg/ml, at most 5 μg/ml). In certain embodiments, the nanodispersion includes from 1 to 100 (e.g., from 1 to 75, from 1 to 50, from 1 to 25, from 10 to 100, from 10 to 75, from 10 to 50, from 10 to 20) μg of metal-containing material per one ml nanodispersion.

In certain embodiments, the nanodispersion containing the material is contacted with the subject relatively soon after formation of the nanodispersion. For example, the nanodispersion containing the material can be contacted with the subject within about one minute or less (e.g., within about 30 seconds or less, within about 10 seconds or less) of forming the nanodispersion. In some embodiments, a longer period of time lapses before the nanodispersion containing the material is contacted with the subject. For example a period of time of at least about 1.5 minutes (e.g., at least about five minutes, at least about 10 minutes, at least about 30 minutes, at least about one hour, at least about 10 hours, at least about a day, at least about a week) lapses between the time the solution containing the material is formed and the nanodispersion containing the material is contacted with the subject.

In some embodiments, the metal-containing material can be in the form of a foam, a spray, or a drop. The foam, spray, or drop can have the same composition as a nanodispersion.

Solutions

In some embodiments, the metal-containing material is in the form of a solution including dissolved metal species. The dissolved metal species can be ionic. The solution is relatively free of particulates having a size greater than one nm. The solution can be formed by dissolving a free standing powder of the material in a solvent for the powder, and filtering the mixture through a filter (e.g., a 0.1 micron filter, a 0.2 micron filter). As an example, a container (e.g., a tea bag-type container) with the free standing powder within it can be immersed in the water or solvent and the resulting solution can be filtered. As another example, a substrate (e.g., in the form of a strip or a bandage) carrying the material can be immersed in the solvent to disperse the metal-containing material. The solvent containing the substrate can be shaken in a shaking incubator (e.g., at 180 RPM and 37° C. for 30 minutes) and/or stirred, then filtered.

The solution includes a solvent that can be an aqueous or an organic solvent. For example, the solvent can be an alcohol (e.g., propanol, ethanol) or an organic solvent. As an example, the solvent can be carbonated water, which can be prepared by sparging CO₂ through water using, for example, a CO₂ Soda Syphon charger. The pH of the solution can be lowered by adding CO₂ to the solution to form carbonic acid. In some embodiments, the solvent is a buffer, such as a lactate buffer, an EDTA buffer, a citrate buffer, a glycolate buffer, a gluconate buffer. The buffer can have a pH at least 3 (e.g., at least 4, at least 5, at least 6, at least 7, or at least 8) and/or at most 9 (e.g., at most 8, at most 7, at most 6, at most 5, or at most 4). In some embodiments, the buffer has a pH of from 3 to 9 (e.g., from 4 to 8, from 3 to 7, from 4 to 6, from 5 to 7). The buffer concentration can be at least 0.05 M (e.g., at least 0.1 M, at least 0.2 M, at least 0.3 M, at least 0.4 M, at least 0.5 M, at least 0.7M) and/or at most one M (e.g., at most 0.7 M, at most 0.5 M, at most 0.4 M, at most 0.3 M, at most 0.2 M, at most 0.1 M). In some embodiments, lowering the pH of the solution (e.g., to less than about 6.5, such as from about 3.5 to about 6.5) can allow for a higher concentration of the dissolved material and/or a faster rate of dissolution.

In some embodiments, the solution contains at least 0.00001 percent by weight (e.g., greater than 0.0001 percent by weight, at least 0.001 percent by weight, at least 0.01 percent by weight, at least 0.1 percent by weight, at least one percent by weight, at least two percent by weight, at least two percent by weight, at least three percent by weight, at least four percent by weight) and/or at most five percent by weight (e.g., at most four percent by weight, at most three percent by weight, at most two percent by weight, at most one percent by weight, at most 0.1 percent by weight, at most 0.01 percent by weight, at most 0.001 percent by weight, at most 0.0001 percent by weight) of the metal-containing material. In some embodiments, the solution includes from 0.00001 to five (e.g., from 0.0001 to five, from 0.001 to five, from 0.01 to five, from 0.1 to five, from one to five, from 0.0001 to three, from 0.001 to three, from 0.01 to three, from 0.1 to three, from one to three, from 0.0001 to one, from 0.001 to one, from 0.01 to one, from 0.1 to one, from 0.1 to 0.5) percent by weight of one or more metal-containing materials. In some embodiments, the solution includes the metal-containing material at a concentration of at least 1 μg/ml (e.g., at least 5 μg/ml, at least 10 μg/ml, at least 20 μg/ml, at least 30 μg/ml, at least 40 μg/ml, at least 50 μg/ml, at least 75 μg/ml) and/or at most 100 μg/ml (e.g., at most 75 μg/ml, at most 50 μg/ml, at most 40 μg/ml, at most 30 μg/ml, at most 20 μg/ml, at most 10 μg/ml, at most 5 μg/ml). In certain embodiments, the solution includes from 1 to 100 (e.g., from 1 to 75, from 1 to 50, from 1 to 25, from 10 to 100, from 10 to 75, from 10 to 50, from 10 to 20) μg of metal-containing material per one ml of the solution.

In certain embodiments, the solution containing the material is contacted with the subject relatively soon after formation of the solution. For example, the solution containing the material can be contacted with the subject within about one minute or less (e.g., within about 30 seconds or less, within about 10 seconds or less) of forming the solution. In some embodiments, a longer period of time lapses before the solution containing the material is contacted with the subject. For example a period of time of at least about 1.5 minutes (e.g., at least about five minutes, at least about 10 minutes, at least about 30 minutes, at least about one hour, at least about 10 hours, at least about a day, at least about a week) lapses between the time the solution containing the material is formed and the solution containing the material is contacted with the subject.

In some embodiments, the metal containing material can be in the form of a foam, a spray, or a drop. The foam, spray, or drop can have the same composition as a solution.

Freeze-Dried Powders

In some embodiments, the metal-containing material can be a freeze-dried powder, formed from freeze-drying a nanodispersion of the metal-containing material that further includes a bulking agent (e.g., mannitol, glycine, gelatin, dextran, glucose, sucrose, and/or lactose) and/or a cryoprotectant (e.g., glycine, glucose, fructose, sucrose, lactose). Without wishing to be bound by theory, it is believed that a bulking agent decreases the likelihood of particle agglomeration, which can occur at high particle concentrations as the solvent is removed by freeze-drying. A cryoprotectant decreases the likelihood of formation of water crystals, which can push the particles into close proximity and increase the likelihood of particle agglomeration. In some embodiments, the freeze-dried powder can be reconstituted into a suspension and/or nanodispersion, for example, by adding water or an aqueous solution and/or by ultrasonicating. In some embodiments, the freeze-dried powder can be incorporated into a pill, capsule, or tablet.

In some embodiments, the freeze-dried powder includes at least 0.01 percent (e.g., at least 0.1 percent, at least one percent, at least five percent, at least ten percent, at least 20 percent, at least 30 percent, at least 40 percent) by weight and/or at most 50 percent (e.g., at most 40 percent, at most 30 percent, at most 20 percent, at most ten percent, at most five percent, at most one percent, at most 0.1 percent) by weight of one or more metal-containing materials. In some embodiments, the freeze-dried powder includes from 0.01 to 50 (e.g., from 0.01 to 40, from 0.01 to 20, from one to 20, from one to 40, from ten to 50, from ten to 30, from 20 to 50, from 20 to 30) percent by weight of one or more metal-containing materials.

In some embodiments, the freeze-dried powder includes at least 35 percent by weight (e.g., at least 40 percent, at least 50 percent, at least 60 percent, at least 70 percent, at least 80 percent, at least 90 percent) and/or at most 99.99 percent (e.g., at most 90 percent, at most 80 percent, at most 70 percent, at most 60 percent, at most 50 percent, at most 40 percent) by weight of one or more stabilizing agents. In some embodiments, the freeze-dried powder includes from 35 to 99.99 (e.g., from 40 to 80, from 40 to 70, from 50 to 60, from 50 to 75, from 50 to 80) percent by weight of one or more stabilizing agents.

In some embodiments, the freeze-dried powder includes at least 0.01 percent (e.g., at least 0.1 percent, at least one percent, at least five percent, at least ten percent) by weight and/or at most 15 percent (e.g., at most ten percent, at most five percent, at most one percent, at most 0.1 percent) by weight of one or more bulking agents and/or cryoprotectants. In some embodiments, the freeze-dried powder includes from 0.01 to 15 (e.g., from 0.01 to ten, from 0.01 to five, from one to 15, from one to ten, from ten to 15) percent by weight of one or more bulking agents and/or cryoprotectants.

Suppositories

In some embodiments, the metal-containing material is in the form of a suppository. The suppository can melt at a physiological temperature and release the metal-containing material at an appropriate location. The suppository can include a suppository base that can melt at physiological temperatures, such as cocoa butter or a hard fat. The suppository also can include a metal-containing material. The metal-containing material can be in the form of a free standing powder or a freeze-dried powder. In some embodiments, the suppository includes at least 70 percent by weight (e.g., at least 75 percent by weight, at least 80 percent by weight, at least 90 percent by weight, at least 95 percent by weight, at least 97 percent by weight) and/or at most 99.99 percent by weight (at most 97 percent by weight, at most 95 percent by weight, at most 90 percent by weight, at most 80 percent by weight, at most 75 percent by weight) of a suppository base. In some embodiments, the suppository includes from 70 to 99.99 (e.g., from 70 to 95, from 70 to 90, from 80 to 95, from 80 to 90) percent by weight of a suppository base. In some embodiments, the suppository includes at least 0.01 percent by weight (e.g., at least 3 percent by weight, at least 5 percent by weight, at least 10 percent by weight, at least 20 percent by weight, at least 25 percent by weight) and/or at most 30 percent by weight (e.g., at most 25 percent by weight, at most 20 percent by weight, at most 10 percent by weight, at most 5 percent by weight, at most 3 percent by weight) of the metal-containing material. In some embodiments, the suppository includes from 0.01 to 30 (e.g., from 0.01 to 25, from 0.01 to 20, from 0.01 to 10, from three to 20, from three to 30, from three to 10, from 10 to 30, from 10 to 20, from 15 to 20) percent by weight of a suppository base.

Formulation Characteristics

While a cream, a nanodispersion, a solution, a freeze-dried powder, and a suppository have been described in the foregoing, in certain embodiments, the formulation can be in the form of a lotion, a gel, a paste, or an ointment. The lotion can have a lower viscosity than a cream; the gel can be transparent, translucent, and/or opaque, the paste can have more solids than a cream; and an ointment can have low levels of water or be substantially free of water (e.g., about 80% free of water, about 90% free of water, about 95% free of water, about 98% free of water, about 99% free of water, 100% free of water).

In some embodiments, various formulations can optionally include one or more components which can be biologically active or biologically inactive. Examples of components are described above. Further examples of such optional components include base components (e.g., water and/or an oil, such as liquid paraffin, vegetable oil, peanut oil, castor oil, cocoa butter), thickening agents (aluminum stearate, hydrogenated lanolin), gelling agents, dispersing agents, suspending agents, thickening agents, coloring agents, perfumes, excipients (starch, tragacanth, cellulose derivatives, silicones, bentonites, silicic acid, talc), foaming agents (e.g., surfactants), surface active agents, preservatives (e.g., methyl paraben, propyl paraben, benzyl alcohol), and cytoconductive agents (e.g., betaglucan). In certain embodiments, a pharmaceutical carrier composition can include a constituent (e.g., DMSO) to assist in the penetration of skin. In some embodiments, a formulation can include tinting agents, emollients, skin conditioning agents, humectants, preservatives, antioxidants, perfumes, chelating agents: physically and chemically compatible with other components of the composition.

In some embodiments, the metal-containing material can be a preservative. In such embodiments, a form or formulation containing the metal-containing material can be prepared with or without additional preservatives. Moreover, in embodiments in which the metal-containing material acts as a preservative, the metal-containing material may be included in a therapeutic formulation containing other therapeutic agents (e.g., the metal-containing material may be included primarily in certain therapeutic compositions to act as a preservative). Formulations can contain the metal-containing preservative at a concentration of at least 0.01 percent (e.g., at least 0.02 percent, at least 0.05 percent, at least 0.1 percent, at least percent) and/or at most one percent (at most 0.1 percent, at most 0.05 percent, at most 0.02 percent). In some embodiments, formulations can contain the metal-containing preservative at a concentration of from 0.01 to one (e.g., from 0.01 to 0.5, from 0.01 to 0.05, from 0.1 to one, from 0.1 to 0.5) percent.

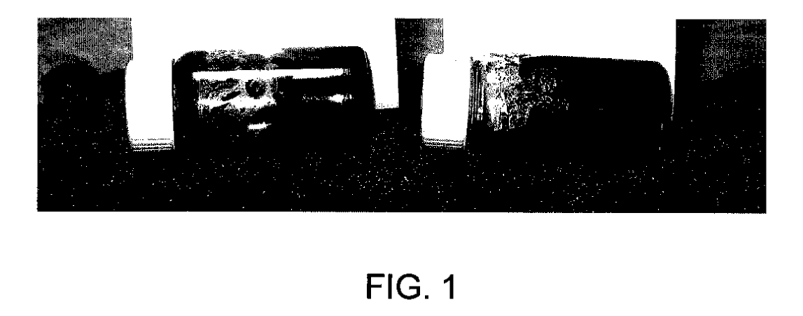

In some embodiments, the formulation decreases the likelihood of discoloration, mirror formation (e.g., silver-mirror formation), and/or viscosity changes, which can occur over time (e.g., one day, two days, five days, one month, two months, three months, six months, a year) when the metal-containing material is mixed with a number of excipients. Without wishing to be bound by theory, it is believed that certain metal ions (e.g., silver ions) are reactive and photosensitive, and can be incompatible with certain excipients and/or form unstable mixtures when incorporated into a formulation. For example, it is believed that in some embodiments, excipients including vicinal diols can induce discoloration by reacting with metals. As an example, a formulation including a metal-containing material (e.g., a silver-containing material) and carboxymethyl cellulose (CMC) can result in discoloration to a dark-colored gel, or a silver-mirror. Referring to FIG. 1, a formulation including a nanocrystalline silver, CMC, and propylene glycol (PG) can result in a silver mirror when stored at 40° C. over three weeks. A silver-containing material mixed with CMC and PEG 400 at pH 7.3, stored at 40° C. over three weeks can result in a dark brown formulation.

In some embodiments, the metal-containing material has a dark color, such as a dark brown to black color. Depending on the components of the formulation, the cream, foam, gel, lotion, paste, ointment, nanodispersion, solution, spray, drop, or suppository containing the material can be lighter in color than the metal-containing material. For example, a cream containing 2.0% of a nanocrystalline silver material can have a grey color.

In some embodiments, the formulations including the metal-containing material is non-staining to fabrics and/or is easily removed from fabrics. This can be advantageous in order to avoid permanent staining of clothes, for example, when the formulation is for topical use on the skin. The non-staining property is assessed by visually comparing photographs of a fabric prior to staining, after application of a formulation, and after washing the fabric; by measuring a lightness factor of a fabric sample prior to staining with a formulation, after staining, and after washing of the fabric sample using a spectrophotometer (e.g., Color Quest XE, Hunter Associates Laboratory, Inc.); and/or by measuring the level of a metal-containing material remaining in the fabric by analyzing the fabric after laundering using atomic absorption spectroscopy after acid digestion of the fabrics. In some embodiments, a non-staining formulation is such that a fabric stained with the formulation can recover at least 70% (e.g., at least 80%, at least 90%, or at least 95%) of the initial lightness factor, prior to staining.

In some embodiments, depending on the condition to be treated, a cream, lotion, gel, solution, nanodispersion, and/or ointment containing the material can be topically applied, for example, to an area of the skin to relieve skin conditions, such as eczema and/or dry skin conditions.

In some embodiments, depending on the condition to be treated, a solution and/or a nanodispersion containing the material can contact an area having mucous membranes such as mouth, eyes, colon, lungs, and/or other organs, in the form of a rinse, a bath, a wash, an enema, a gargle, a spray, and/or drops, with or without the use of a device. As an example, the solution and/or the nanodispersion can be injected into a subject using a small needle injector and/or a needleless injector. As an another example, the solution and/or the nanodispersion containing the material can be formed into an aerosol (e.g., an aerosol prepared by a mechanical mister, such as a spray bottle or a nebulizer), and the aerosol can be contacted with the subject using an appropriate device (e.g., a hand held inhaler, a mechanical mister, a spray bottle, a nebulizer, an oxygen tent). As a further example, a solution and/or nanodispersion containing the material can be contacted with the subject via a catheter.

In some embodiments, the metal-containing material is in the form of an aerosol or dry powder, formed from lyophilizing, freeze-drying, or drying a nanodispersion. The aerosol or dry powder can be inhaled to contact a respiratory area such as the mouth, lungs, or nasal passage for treatment of respiratory conditions. In some embodiments, the metal-containing material is sub-micron in size.

In some embodiments, the metal-containing material in the form of an article such as a suppository, solution, nanodispersion, tablet, capsule, pill, or foam can contact the gastrointestinal system of a subject to treat, for example, inflammatory bowel disease (IBD). The article can include a sustained release formulation (e.g., a sustained release capsule) which can allow the metal-containing material to be released at a predetermined rate (e.g., a relatively constant rate). In some embodiments, an article can include a material (e.g., in the form of a coating and/or in the form of a matrix material) that allows the article to pass through certain portions of the gastrointestinal system with relatively little (e.g., no) release of the metal-containing material, but that allows a relatively large amount of the metal-containing material to be released in a desired portion of the gastrointestinal system. As an example, the article can be an enteric article (e.g., an enteric coated tablet, an enteric coated capsule, an enteric coated pill) so that the formulation passes through the stomach with little (e.g., no) metal-containing material being released, and so that the metal-containing material is relatively easily released by the article in the intestines. In some embodiments, the article can be an enema or a suppository, which can contact the gastrointestinal system (e.g., the colon) to provide a therapeutic effect.

In some embodiments, the metal-containing formulation (e.g., a cream) is an anti-microbial barrier. In some embodiments, the metal-containing formulation is anti-inflammatory and reduces inflammation in a subject, for example, by suppressing the expression of inflammatory cells. The metal-containing formulation can have enhanced emollient properties such that the formulation can soften and soothe the skin when applied locally. An emollient property is assessed by measuring the extent to which a formulation decreases water evaporation (e.g., from skin). The metal-containing formulation can be substantially free of steroids. The metal-containing formulation can be non-allergenic (e.g., non-allergenic to nuts). In some embodiments, a formulation without metal-containing material, such as a cream, has moisturizing and protecting properties, which can provide therapeutic effects when applied onto an area of a subject. The moisturizing property is measured by a transepidermal water loss (TEWL) test using healthy volunteers (Hill-Top Research, Manchester, UK). In some embodiments, water loss is measured using a Vapometer (Delfin Technologies Ltd., Finland).

In some embodiments, the metal containing formulation has good spreadability, such that the formulation can be spread into a thin layer when topically applied before drying. The spreadability can depend on the viscosity, the melting temperature, the evaporation rate, and/or the solid content of the formulation. For example, in some embodiments, a low viscosity formulation (e.g., viscosity of less than 45,000 cPs) can have a large spreadability and a watery feeling when rubbed into an area of the skin, and a high viscosity (e.g., greater than 2,000,000 cPs) can limit the spreadability of the formulation. The metal-containing formulation can have a viscosity of greater than 60,000 cPs (e.g., greater than 100,000 cPs, greater than 200,000 cPs, greater than 400,000 cPs, greater than 600,000 cPs, greater than 800,000 cPs, greater than 1,000,000 cPs, greater than 1,200,000 cPs, greater than 1,400,000 cPs, or greater than 1,600,000 cPs) and/or at most 2,000,000 cPs (e.g., less than 1,800,000 cPs, less than 1,600,000 cPs, less than 1,400,000 cPs, less than 1,200,000 cPs, less than 1,000,000 cPs, less than 800,000 cPs, less than 600,000 cPs, less than 400,000 cPs, less than 200,000 cPs, or less than 100,000 cPs). Viscosity is measured using a viscometer (e.g., a Brookfield RV II pro viscometer with T-D spindle measured at 1.0 rpm). In some embodiments, a low melting temperature, a low evaporation rate, and/or a low solid content can increase the spreadability of a formulation.

In some embodiments, a metal-containing formulation including metal-containing particles of small particle size (e.g., about 400 nm or less, about 300 nm or less, about 200 nm or less, about 150 nm or less, about 100 nm or less, about 50 nm or less, or about 25 nm or less; and/or about 10 nm or more, about 25 nm or more, about 50 nm or more, about 100 nm or more, about 150 nm or more, about 200 nm or more, or about 300 nm or more) can be more therapeutically effective (e.g., 2× more effect, 5× more effective, 10× more effective, 20× more effective, 50× more effective, 100× more effective) than a metal-containing formulation that does not include metal-containing particles of small particle size, such that a smaller quantity of metal-containing material (e.g., 1/100 of a quantity, 1/50 of a quantity, 1/20 of a quantity, 1/10 of a quantity, ⅕ of a quantity, ½ of a quantity) is needed to achieve the same therapeutic effect when the formulation is administered to a subject, for example, to an open wound, past the skin barrier, and/or to a mucosal or serosal area. A decreased quantity of a metal-containing material in a formulation can have decreased toxicological effect on a subject, and can facilitate the administration of a formulation.

In some embodiments, when applied to an area of a subject, the formulation can release a steady amount of a therapeutic agent (e.g., a metal-containing material) over a period of time (e.g., at least 30 minutes, at least one hour, at least two hours, at least three hours, at least six hours, at least 12 hours, or at least 24 hours; and/or at most 48 hours, at most 24 hours, at most 12 hours, at most six hours, at most three hours, at most two hours, or at most one hour). In some embodiments, the period of time is from 30 minutes to 48 hours (e.g., from 30 minutes to 24 hours, from one hour to 24 hours, from six hours to 24 hours). A steady amount refers to an amount that varies by less than 90% (less than 80%, less than 70%, less than 60%) of the initial amount over the period of time.

Conditions

The metal-containing compound can be used to treat one or more conditions. In some embodiments, the conditions that are treated with the metal-containing materials are mucosal or serosal conditions, skin conditions, respiratory conditions, musculo-skeletal conditions, and/or circulatory conditions. The conditions can be caused by bacteria, inflammation, hyperproliferation, fungi, viruses, protozoa, autoimmune responses, or toxic or damaging substances produced by bacteria, virus, fungi, or protozoa. In some embodiments, the conditions are idiopathic in nature.

The conditions could be the same type of condition (e.g., multiple skin or integument conditions) or different types of conditions. For example, a cream containing an appropriate metal-containing material (e.g., antimicrobial, atomically disordered, silver-containing material) can be applied to an area of the skin having multiple skin or integument conditions (e.g., a burn and psoriasis) so that the metal-containing material treats the multiple skin or integument conditions.

Moreover, while the foregoing has described embodiments that involve one method of contacting a subject with the metal-containing material, in other embodiments, more than one method of contacting a subject with the metal-containing material can be used. For example, the methods can include one or more of ingestion (e.g., oral ingestion), injection (e.g., using a needle, using a needleless injector), topical administration, inhalation (e.g., inhalation of a dry powder, inhalation of an aerosol) and/or application of a dressing. The methods for application of material to the subject can vary in a number of ways, generally depending upon the form of the material as applied and/or the location of the condition to be treated. In general, the amount of material used is selected so that the desired therapeutic effect (e.g., reduction in the condition being treated) is achieved while the material introduces an acceptable level of toxicity (e.g., little or no toxicity) to the subject. Generally, the amount of the material used will vary with the conditions being treated, the stage of advancement of the condition, the age and type of host, and the type, concentration and form of the material as applied. Appropriate amounts in any given instance will be readily apparent to those skilled in the art or capable of determination by routine experimentation. In some embodiments, a single application of the material may be sufficient. In certain embodiments, the material may be applied repeatedly over a period of time, such as several times a day for a period of days, weeks, months or years.

Furthermore, while the foregoing has described embodiments in which one form of the metal-containing material is used, in other embodiments, more than one form of the metal-containing material can be used. For example, the methods can include using the metal-containing material in the form of a coating (e.g., a dressing), a free standing powder, a freeze-dried powder, a solution and/or a pharmaceutical carrier composition.

Moreover, the metal-containing material can be used in various industrial applications. For example, the metal-containing material can be used to reduce and/or prevent microbial growth on industrial surfaces (e.g., industrial surfaces where microbial growth may occur, such as warm and/or moist surfaces). Examples of industrial surfaces include heating pipes and furnace filters. In certain embodiments, the metal-containing material can be disposed (e.g., coated or sprayed) on the surface of interest to reduce and/or prevent microbial growth and/or formation of biofilms. This can be advantageous in preventing the spread of microbes via, for example, heating and/or air circulation systems within buildings.

Biofllm Conditions

In some embodiments, the condition is a microbial condition caused by Gram-positive, Gram-negative, fungal pathogens, and antibiotic-resistant bacteria. An antibiotic-resistant bacterium refers to a bacterium whose growth and reproduction is unaffected by particular antibiotics such as methicillin and/or vancomycin. In some embodiments, the conditions are characterized by the presence of bacterial biofilms. A biofilm is a complex aggregation of bacteria, which secrete a protective and adhesive matrix. Biofilms can attach to a surface, and exhibit structural heterogeneity, genetic diversity, complex community interactions, and an extracellular matrix of polymeric substances. Biofilm conditions can be the cause of persistent and chronic infections, and can affect a variety of tissues such as the gum and jawbone (periodontal tissue), the eye (e.g., infection by contact lenses having biofilms), the lung (e.g., chronic lung infections), the gastrointestinal tract, internal tissue (e.g., endocarditis) and the skin (e.g., infected skin, infected burn wounds). Biofilms can also form on medical devices implanted in the body such as catheters and heart valves, or on contact lenses.

In general, treatment and/or inhibition of bacterial biofilm conditions involves contacting the metal-containing material with the area of the body having the condition. As an example, a biofilm condition can be inhibited and/or treated by contacting the area having the condition with a formulation such as a cream, foam, gel, lotion, paste, ointment, nanodispersion, and/or solution containing the metal-containing material. In some embodiments, treatment and/or inhibition of bacterial biofilm conditions can involve contacting the metal-containing material with the area of the body having the condition in combination with another type of antibacterial agent, for example an antibiotic. Treatment with the antibacterial agent different from the metal-containing material can occur before, after, or simultaneously with, the treatment with metal-containing material.

The formulation(s) can be administered to a subject (e.g., a human subject) at a dosage of at least one dose per day (e.g., at least one dose per 12 hours, at least one dose per 6 hours) and/or at most one dose per three hours (e.g., at most one dose per six hours, at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours). The formulation(s) can contact the area continuously, or for a duration of about one hour per dose (e.g., about a half hour per dose, about 15 minutes per dose, about five minutes per dose, about one minute per dose). In some embodiments, the formulation(s) such as a nanodispersion/solution contains the metal-containing material at a concentration of at least 1 μg metal-containing material per one ml of the nanodispersion/solution (e.g., at least 5 μg/ml, at least 10 μg/ml, at least 20 μg/ml, at least 30 μg/ml, at least 40 μg/ml, at least 50 μg/ml, at least 75 μg/ml) and/or at most 100 μg/ml (e.g., at most 75 μg/ml, at most 50 μg/ml, at most 40 μg/ml, at most 30 μg/ml, at most 20 μg/ml, at most 10 μg/ml, at most 5 μg/ml). In certain embodiments, the nanodispersion includes from 1 to 100 (e.g., from 1 to 75, from 1 to 50, from 1 to 25, from 10 to 100, from 10 to 75, from 10 to 50, from 10 to 20) μg of metal-containing material per one ml nanodispersion/solution. In some embodiments, the concentration of the formulation is less than or equal to the minimum inhibitory concentration for a bacteria of the biofilm.

In some embodiments, the formation of bacterial biofilms can be prevented by contacting the metal-containing material with the area of the body or device susceptible to the formation of a biofilm. As an example, a biofilm condition can be prevented by contacting or coating the susceptible area or device with a formulation such as a cream, foam, gel, lotion, paste, ointment, nanodispersion, and/or solution containing the metal-containing material. The formulation can be administered to a subject (e.g., a human subject) at a dosage of at least one dose per day (e.g., at least one dose per 12 hours, at least one dose per 6 hours) and/or at most one dose per three hours (e.g., at most one dose per six hours, at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours). The formulation can contact the area continuously, or for a duration of about one hour (e.g., about a half hour, about 15 minutes, about five minutes, about one minute).

Mucosal or Serosal Conditions

In some embodiments, the mucosal or serosal condition is inflammatory bowel disease (IBD). IBD is a chronic inflammatory condition affecting the gastrointestinal tract. IBD includes two major categories: ulcerative colitis and Crohn's disease. Without wishing to be bound by theory, it is believed that the cause of IBD is not clearly known but there may be several factors, such as environmental factors, microbial pathogens, immuno-regulation defects, antigens, inflammatory mediators, and production of nitric oxides. It is believed that many cytokines are involved in the pathogenesis of IBD: IL-1, IL-6, IL-8, TNF-α, IL-12, IL-13, IL-15, IL-16, IL-17, IL-18, IL-23, and IL-25. In some embodiments, it is believed that metal-containing materials can suppress TNF-α0 and IL-12B expression of inflammatory cells.

IBD can be treated using a suppository, by an enema, and/or by orally administering a formulation of metal-containing material. For example, the formulation can be a nanodispersion, a tablet, a pill, a capsule, or a suppository having a metal-containing material administered at a metal dosage of at least 0.4 mg of a metal-containing material per one kg of a subject (e.g., at least 0.4 mg/kg, at least 40 mg/kg, at least 400 mg/kg) and/or at most 1000 mg/kg (e.g., at most 400 mg/kg, at most 40 mg/kg, at most 0.4 mg/kg), and for a period sufficient to treat/alleviate/cure the condition. In some embodiments, the metal dosage is from 0.4 to 1000 mg (e.g., from 0.4 to 400 mg, from 0.4 to 40 mg, from 40 to 400 mg) of metal-containing material per one kg of a subject. In some embodiments, for a 70 kg person, at least 20 mg (e.g., at least 50 mg, at least 100 mg, at least 200 mg, at least 300 mg, at least 400 mg, or at least 500 mg) and/or at most 600 mg (e.g., at most 500 mg, at most 400 mg, at least 300 mg, at least 200 mg, at least 100 mg, at least 50 mg) of a metal-containing material is administered per single dose, or from 20 to 600 mg (e.g., from 50 to 500 mg, from 50 to 400 mg, from 20 to 400 mg, from 20 to 300 mg, from 20 to 200 mg) of a metal-containing material is administered per single dose, which can take the form of an enema having a volume of about 60 ml (about 30 ml, about 40 ml, about 50 ml, about 70 ml, about 80 ml, or about 90 ml). The formulation can be administered to a subject at a dosage of at least one dose per day (e.g., at least one dose per 12 hours, at least one dose per 6 hours) and/or at most one dose per three hours (e.g., at most one dose per six hours, at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours).

In some embodiments, the mucosal or serosal condition is a bacterial mucosal or serosal condition, a biofilm mucosal or serosal condition, a microbial mucosal or serosal condition, an inflammatory mucosal or serosal condition, a fungal mucosal or serosal condition, a viral mucosal or serosal condition, an autoimmune mucosal or serosal condition, an idiopathic mucosal or serosal condition, a hyperproliferative mucosal or serosal condition, a cancerous mucosal, and/or serosal condition. Examples of mucosal or serosal conditions include pericarditis, Bowen's disease, stomatitis, prostatitis, sinusitis, allergic rhinitis, digestive disorders, peptic ulcers, esophageal ulcers, gastric ulcers, duodenal ulcer, esophagitis, gastritis, enteritis, enterogastric intestinal hemorrhage, toxic epidermal necrolysis syndrome, Stevens Johnson syndrome, fibrotic condition (e.g., cystic fibrosis), bronchitis, pneumonia (e.g., nosocomial pneumonia, ventilator-assisted pneumonia), pharyngitis, common cold, ear infections, sore throat, sexually transmitted diseases (e.g., syphilis, gonorrhea, herpes, genital warts, HIV, chlamydia), inflammatory bowel disease (IBD), colitis, hemorrhoids, thrush, dental conditions, oral conditions, eye conditions (e.g., conjunctivitis, eye infections caused by infected contact lens), and periodontal conditions. Generally, the treatment of mucosal or serosal conditions involves contacting the metal-containing material with the area of a mucosal or serosal region having the condition. Mucosal or serosal areas include, for example, the oral cavity, the nasal cavity, the colon, the small intestine, the large intestine, the stomach, and the esophagus. As an example, certain respiratory mucosal or serosal conditions can be treated by inhaling a powder (e.g., a freeze-dried powder, a free standing powder) of the metal-containing material (e.g., with a dry powder inhaler). As another example, certain respiratory mucosal or serosal conditions can be treated by inhaling an aerosol containing the metal-containing material (e.g., with an inhaler). As another example, certain mucosal or serosal conditions (e.g., eye conditions) can be treated by contacting an affected organ (e.g., an eye) with a solution and/or a nanodispersion of the metal-containing material in the form of, for example, a spray, a drop, and/or a wash. In embodiments where the infection is associated with the use of a device (e.g. contact lenses), treatment can include contacting the device with a solution and/or nanodispersions of the metal-containing material. As an additional example, certain mucosal or serosal conditions (e.g., oral mucosal or serosal conditions) can be treated by gargling or spraying a solution of the metal-containing material.

Skin or Integument Conditions

Generally, the treatment of skin or integument conditions involves contacting the metal-containing material with the area of the skin having the condition. As an example, a skin or integument condition can be treated by contacting the area of skin having the condition with a dressing having a coating of the metal-containing material. As another example, a skin or integument condition can be treated by contacting the area of skin having the condition with a nanodispersion/solution containing the metal-containing material. As an additional example, a skin or integument condition can be treated by contacting the area of skin having the condition with a pharmaceutical carrier composition containing the metal-containing material, such as a cream, foam, gel, lotion, paste, ointment, nanodispersion and/or solution. In the case of onychomycosis, the material may be applied to the nail in an appropriate form such that the material penetrates the hard nail to contact the affected area. Treatment can continue until the condition is cured or ameliorated, In some embodiments, treatment includes a dosage of at least one dose per day (e.g., at least one dose per 12 hours, at least one dose per six hours, at least one dose per three hours) and/or at most one dose per hour (e.g., at most one dose per three hours, at most one dose per six hours, at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours). In some embodiments, a dose includes at least 0.2 gram (e.g., at least 0.4 gram, at least 0.6 gram, at least 0.8 gram, at least one gram, at least two grams, at least three grams, at least four grams) and/or at most five grams (e.g., at most four grams, at most three grams, at most two grams, at most one gram, at most 0.8 gram, at most 0.6 gram, or at most 0.4 gram) of a formulation per area of about 200 cm² (e.g., about 150 cm², about 100 cm², about 80 cm², about 60 cm², about 40 cm², about 20 cm², or about 10 cm²). In some embodiments, a dose includes from 0.2 gram to five grams (e.g., from 0.4 to four grams, from 0.3 to three grams, from one to four grams, from one to three grams, from one to two grams) of a formulation per area of about 200 cm² (e.g., about 150 cm², about 100 cm², about 80 cm², about 60 cm², about 40 cm², about 20 cm², or about 10 cm²).

The skin condition or an integument condition can be a bacterial skin condition, a biofilm skin condition, an inflammatory skin condition, a hyperproliferative skin condition, a fungal skin condition, a viral skin condition, an autoimmune skin condition, an idiopathic skin condition, a hyperproliferative skin condition, a cancerous skin condition, a microbial integument condition, an inflammatory integument condition, a fungal integument condition, a viral integument condition, a protozoal skin condition, an autoimmune integument condition, an idiopathic integument condition, a hyperproliferative integument condition, and/or a cancerous integument condition. Examples of skin conditions or integument conditions include burns, eczema (e.g., atopic eczema, atopic dermatis, acrodermatitis continua, contact allergic dermatitis, contact irritant dermatitis, dyshidrotic eczema, pompholyx, lichen simplex chronicus, nummular eczema, seborrheic dermatitis, stasis eczema, eczematous dermatitis), erythroderma, insect bites, mycosis fungoides, pyoderma gangrenosum, erythema multiforme, rosacea, onychomycosis, acne (e.g., acne vulgaris, neonatal acne, infantile acne, pomade acne), psoriasis, Reiter's syndrome, pityriasis rubra pilaris, hyperpigmentation, vitiligo, scarring conditions (e.g., hypertropic scarring), keloids, lichen planus, pruritis, itching, ichtyosis, ulcer and erosion due to cutaneous trauma (e.g., diabetic foot ulcer), dry skin, epidermolysis bullosa, age-related skin disorders (e.g., wrinkles, cellulite, cutaneous changes of intrinsic or extrinsic aging) and hyperproliferative skin disorders, such as, for example, hyperproliferative variants of the disorders of keratinization (e.g., actinic keratosis, senile keratosis).

Respiratory Conditions

In general, the treatment of respiratory conditions involves contacting the metal-containing material with the area of the respiratory system having the condition. Areas of the respiratory system include, for example, the oral cavity, the nasal cavity, and the lungs. As an example, certain respiratory conditions can be treated by inhaling a free standing powder and/or a freeze-dried powder of the metal-containing material (e.g., with a dry powder inhaler). As another example, certain respiratory conditions can be treated by inhaling a nanodispersion and/or solution containing the metal-containing material (e.g., in the form of an aerosol with an inhaler). In some embodiments, treatment includes a dosage of at least one dose per day (e.g., at least one dose per 12 hours, at least one dose per six hours, at least one dose per three hours) and/or at most one dose per hour (e.g., at most one dose per three hours, at most one dose per six hours, at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours). In some embodiments, a dose includes a metal dosage of at least 0.1 milligram (mg) of a metal-containing material per kilogram (kg) of a subject (e.g., at least 0.5 mg/kg, at least one mg/kg, at least five mg/kg, at least 10 mg/kg, at least 50 mg/kg, at least 100 mg/kg, or at least 500 mg/kg) and/or at most 1000 mg/kg (e.g., at most 500 mg/kg, at least 100 mg/kg, at most 50 mg/kg, at most 10 mg/kg, at most five mg/kg, at most one mg/kg, at most 0.5 mg/kg). In some embodiments, a dose includes a metal dosage of from 0.1 mg to 1000 mg (e.g., from 0.1 mg to 500 mg, from one mg to 500 mg, from 10 mg to 500 mg, from 50 mg to 500 mg, from 100 mg to 300 mg, from 100 mg to 200 mg) of a metal-containing material per kilogram (kg) of a subject.

The respiratory condition can be a bacterial respiratory condition, a biofilm respiratory condition, a microbial respiratory condition, an inflammatory respiratory condition, a fungal respiratory condition, a viral respiratory condition, an autoimmune respiratory condition, an idiopathic respiratory condition, a hyperproliferative respiratory condition, a cancerous respiratory condition. Examples of respiratory conditions include asthma, emphysema, bronchitis, pulmonary edema, acute respiratory distress syndrome, bronchopulmonary dysplasia, fibrotic conditions (e.g., pulmonary fibrosis), pulmonary atelectasis, tuberculosis, pneumonia, sinusitis, allergic rhinitis, pharyngitis, mucositis, stomatitis, chronic obstructive pulmonary disease, bronchiectasis, lupus pneumonitis, and/or cystic fibrosis.

Musculo-Skeletal Conditions

Generally, the treatment of musculo-skeletal conditions involves contacting the metal-containing material with the area of the musculo-skeletal system having the condition. Areas of the musculo-skeletal system include, for example, the joints, the muscles, and the tendons. As an example, certain musculo-skeletal conditions can be treated by injecting (e.g., via a small needle injector) a nanodispersion and/or solution containing the metal-containing material into the subject. As another example, certain musculo-skeletal conditions can be treated by injecting (e.g., via a needleless injector) a powder (e.g., a free standing powder, a freeze-dried powder) of the metal-containing material into the subject. As an additional example, certain musculo-skeletal conditions can be treated by using a pharmaceutical carrier composition of the metal-containing material, such as a penetrating pharmaceutical carrier composition of the metal-containing material (e.g., a composition containing DMSO). In some embodiments, treatment includes a dosage of at least one dose per week (e.g., at least one dose per day, at least one dose per 12 hours, at least one dose per six hours, at least one dose per three hours) and/or at most one dose per hour (e.g., at most one dose per three hours, at most one dose per six hours, at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours). In some embodiments, a dose includes a metal dosage of at least 0.1 milligram (mg) of a metal-containing material per kilogram (kg) of a subject (e.g., at least 0.5 mg/kg, at least one mg/kg, at least five mg/kg, at least 10 mg/kg, at least 50 mg/kg, at least 100 mg/kg, or at least 500 mg/kg) and/or at most 1000 mg/kg (e.g., at most 500 mg/kg, at least 100 mg/kg, at most 50 mg/kg, at most 10 mg/kg, at most five mg/kg, at most one mg/kg, at most 0.5 mg/kg). In some embodiments, a dose includes a metal dosage of from 0.1 mg to 1000 mg (e.g., from 0.1 mg to 500 mg, from one mg to 500 mg, from 10 mg to 500 mg, from 50 mg to 500 mg, from 100 mg to 300 mg, from 100 mg to 200 mg) of a metal-containing material per kilogram (kg) of a subject.

In some embodiments, the musculo-skeletal condition is a bacterial musculo-skeletal condition, a biofilm musculo-skeletal condition, a microbial musculo-skeletal condition, an inflammatory musculo-skeletal condition, a fungal musculo-skeletal condition, a viral musculo-skeletal condition, an autoimmune musculo-skeletal condition, an idiopathic musculo-skeletal condition, a hyperproliferative musculo-skeletal condition, and/or a cancerous musculo-skeletal condition. A musculo-skeletal condition can be, for example, a degenerative musculo-skeletal condition (e.g., arthritis) or a traumatic musculo-skeletal condition (e.g., a torn or damaged muscle). Examples of musculo-skeletal conditions include tendonitis, osteomyelitis, fibromyalgia, bursitis and arthritis.

Circulatory Conditions

In general, the treatment of circulatory conditions involves contacting the metal-containing material with the area of the circulatory system having the condition. Areas of the circulatory system include, for example, the heart, the lymphatic system, blood, blood vessels (e.g., arteries, veins). As an example, certain circulatory conditions can be treated by injecting (e.g., via a small needle injector) a nanodispersion and/or a solution containing the metal-containing material into the subject. As another example, certain circulatory conditions can be treated by injecting (e.g., via a needleless injector) a powder (e.g., a freeze-dried powder, a free standing powder) of the metal-containing material into the subject. In some embodiments, treatment includes a dosage of at least one dose per week (e.g., at least one dose per day, at least one dose per 12 hours, at least one dose per six hours, at least one dose per three hours) and/or at most one dose per hour (e.g., at most one dose per three hours, at most one dose per six hours, at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours). In some embodiments, a dose includes a metal dosage of at least 0.1 milligram (mg) of a metal-containing material per kilogram (kg) of a subject (e.g., at least 0.5 mg/kg, at least one mg/kg, at least five mg/kg, at least 10 mg/kg, at least 50 mg/kg, at least 100 mg/kg, or at least 500 mg/kg) and/or at most 1000 mg/kg (e.g., at most 500 mg/kg, at least 100 mg/kg, at most 50 mg/kg, at most 10 mg/kg, at most five mg/kg, at most one mg/kg, at most 0.5 mg/kg). In some embodiments, a dose includes a metal dosage of from 0.1 mg to 1000 mg (e.g., from 0.1 mg to 500 mg, from one mg to 500 mg, from 10 mg to 500 mg, from 50 mg to 500 mg, from 100 mg to 300 mg, from 100 mg to 200 mg) of a metal-containing material per kilogram (kg) of a subject.

In certain embodiments, the circulatory condition is a bacterial circulatory condition, a biofilm circulatory condition, a microbial circulatory condition, an inflammatory circulatory condition, a fungal circulatory condition, a viral circulatory condition, an autoimmune circulatory condition, an idiopathic circulatory condition, a hyperproliferative circulatory condition, and/or a cancerous circulatory condition. As referred to herein, circulatory conditions include lymphatic conditions. Examples of circulatory conditions include arteriosclerosis, lymphoma, septicemia, leukemia, ischemic vascular disease, lymphangitis and atherosclerosis.

Hyperproliferative Conditions

In embodiments in which the metal-containing material is used to treat hyperproliferation of cell growth (e.g., cancerous conditions, such as malignant tumors, or non-cancerous conditions, such as benign tumors), the metal-containing material can be used to modulate matrix metalloproteinases (MMPs) and/or modulates cytokines by contacting affected tissue (e.g., a hyperplastic tissue, a tumor tissue or a cancerous lesion) with the metal-containing material. It has been observed that the metal-containing material (e.g., an antimicrobial, anti-biofilm, antibacterial, anti-inflammatory, antifungal, antiviral, anti-autoimmune, anti-cancer, and/or MMP modulating, nanocrystalline and/or atomically disordered, silver-containing material) can be effective in preventing production of a high number of MMPs and/or cytokines by certain cells without necessarily reducing MMP and/or cytokine production by the same cells to about zero. It is believed, however, that in certain embodiments, the metal-containing material can be used to inhibit MMP and/or cytokine production (e.g., bring MMP and/or cytokine production to normal levels, desired levels, and/or about zero) in certain cells.

MMPs refer to any protease of the family of MMPs which are involved in the degradation of connective tissues, such as collagen, elastins, fibronectin, laminin, and other components of the extracellular matrix, and associated with conditions in which excessive degradation of extracellular matrix occurs, such as tumor invasion and metastasis. Examples of MMPs include MMP-2 (secreted by fibroblasts and a wide variety of other cell types) and MMP-9 (released by mononuclear phagocytes, neutrophils, corneal epithelial cells, tumor cells, cytotrophoblasts and keratinocytes). Cytokine refers to a nonimmunoglobulin polypeptide secreted by monocytes and lymphocytes in response to interaction with a specific antigen, a nonspecific antigen, or a nonspecific soluble stimulus (e.g., endotoxin, other cytokines). Cytokines affect the magnitude of inflammatory or immune responses. Cytokines can be divided into several groups, which include interferons, tumor necrosis factor (TNF), interleukins (e.g., IL-1 to IL-23), transforming growth factors, and the hematopoietic colony-stimulating factors. An example of a cytokine is TNF-α. A fibroblast is an area connective tissue cell which is a flat-elongated cell with cytoplasmic processes at each end having a flat, oval vesicular nucleus. Fibroblasts progenitors which differentiate into chondroblasts, collagenoblasts, and osteoblasts form the fibrous tissues in the body, tendons, aponeuroses, supporting and binding tissues of all sorts. Hyperplastic tissue refers to tissue in which there is an abnormal multiplication or increase in the number of cells in a normal arrangement in normal tissue or an organ. A tumor refers to spontaneous growth of tissue in which multiplication of cells is abnormal, uncontrolled and progressive. A tumor generally serves no useful function and grows at the expense of the healthy organism. A cancerous lesion is a tumor of epithelial tissue, or malignant, new growth made up of epithelial cells tending to infiltrate surrounding tissues and to give rise to metastases. As used in reference to the skin, a cancerous lesion means a lesion which may be a result of a primary cancer, or a metastasis to the site from a local tumor or from a tumor in a distant site. It may take the form of a cavity, an open area on the surface of the skin, skin nodules, or a nodular growth extending from the surface of the skin.

Conditions characterized by undesirable MMP activity include ulcers, asthma, acute respiratory distress syndrome, skin disorders, skin aging, keratoconus, restenosis, osteo- and rheumatoid arthritis, degenerative joint disease, bone disease, wounds, cancer including cell proliferation, invasiveness, metastasis (carcinoma, fibrosarcoma, osteosarcoma), hypovolemic shock, periodontal disease, epidermolysis bullosa, scleritis, atherosclerosis, multiple sclerosis, inflammatory diseases of the central nervous system, vascular leakage syndrome, collagenase induced disease, adhesions of the peritoneum, strictures of the esophagus or bowel, ureteral or urethral strictures, and biliary strictures. Excessive TNF production has been reported in diseases which are characterized by excessive MMP activity, such as autoimmune disease, cancer, cachexia, HIV infection, and cardiovascular conditions.

Mechanism

Without wishing to be bound by theory, it is believed that the therapeutic properties of the metal-containing materials may be explained by one or more potential mechanisms. In one potential mechanism (e.g., at relatively high pH), it is believed that the metal-containing material (e.g., antimicrobial, atomically disordered, nanocrystalline silver-containing materials) forms one or more metastable, relatively high level metal hydroxide species (e.g., Ag(OH)₄³⁻, Ag(OH)₆³⁻) that either directly or indirectly (e.g., via the formation of one or more biological mediators) provide the observed therapeutic properties. In another potential mechanism, it is believed that the metal-containing material is capable of releasing clusters of the metal (e.g., clusters of Ag⁰, clusters of Ag⁺, clusters containing both Ag⁺ and Ag⁰) that provide the observed therapeutic properties. In a further potential mechanism, it is believed that the concentration of silver in a solution can be raised above the saturation concentration of bare silver ions (e.g., to provide a relatively sustaining reservoir of silver as bare silver ions are consumed). It is believed that, as the bare silver ions are consumed, some of the other silver-containing species can decompose to create additional bare silver ions in accordance with chemical equilibria. It is also believed that the presence of silver in one or more forms other than bare silver ions may raise the level for the effective silver concentration that is nonharmful (e.g., non-toxic) to the cells of a subject (e.g., a human). In an additional potential mechanism, it is believed that one or more forms of silver complexes may be capable of penetrating cellular membranes (e.g., by mimicking species that are normally transported through the membranes), which may accelerate the permeation of silver into the cells. In general, it is believed that the form of the silver-containing species contained in an aqueous solution depends on the solution pH and/or the concentrations of the various silver-containing species in the solid form of the silver-containing material. It is believed that, in general, at low pH the dominant species is a bare silver ion, but that at higher pH, where the solubility of bare silver ions is believed to be limited by the solubility of silver hydroxide, other types of species including complexed silver ions and/or silver-containing clusters become increasingly stable provided that the concentration of bare silver ions remains at the saturation concentration. It is also believed that the nature and relative population of the silver-containing species can depend on the rate at which the species can dissolve from the solid silver-bearing material and the rate at which the species can react with one another in the solution. It is believed that combinations of potential mechanisms may result in the observed therapeutic effect of the metal-containing material.

Without wishing to be bound by theory, it is believed that in some embodiments, a metal-containing material can inhibit bacterial matrix formation, for example, in a biofilm. In some embodiments, the metal-containing material can decrease the amount of ATP available to a microbe for inhibition or microbicidal purposes.

In general, clusters refer to relatively small groups of atoms, ions or the like. For example, a cluster can contain at least two (e.g., at least three, at least four, at least five, at least six, at least seven, at least eight, at least nine, at least 10, at least 11, at least 12, at least 13, at least 14, at least 15, at least 20, at least 30, at least 40, at least 50, at least 60, at least 70, at least 80, at least 90) atoms, ions or the like, and/or at most 1,000 (e.g., at most 900, at most 800, at most 700, at most 600, at most 500, at most 400, at most 300, at most 200, at most 100) atoms, ions or the like. Clusters are described, for example, in R. P. Andres et al., “Research Opportunities on Cluster and Cluster-Assembled Materials”, J. Mater. Res. Vol. 4, No 3, 1989, p. 704. In certain embodiments, a cluster (e.g., a cluster containing silver) can contain less than the 14 atoms and have a normal face centered cubic crystal lattice.

Materials

The metal-containing material can be an ionic material or a non-ionic material. The metal-containing material can be, for example, an atom, a molecule, or a cluster. In general, the metal-containing material is a metal or an alloy. Examples of metal elements that can be contained in metal-containing materials include Group I A metal elements (e.g., Li and others), Group II A metal elements (e.g., Be and others), Group III A metal elements (e.g., Sc and others), Group IV A metal elements (e.g., Ti and others), Group V A metal elements (e.g., V and others), Group VI A metal elements (e.g., Cr and others), Group VII A metal elements (e.g., Mn and others), Group VIII A metal elements (e.g., Fe, Co, Ni and others), Group I B metal elements (e.g., Cu and others), Group II B metal elements (e.g., Zn and others), members of the lanthanide metal element series (e.g., La and others), and members of the actinide metal element series (e.g., Ac and others). In certain embodiments, metal-containing materials contain silver, gold, platinum, palladium, iridium, zinc, copper, tin, antimony, and/or bismuth. In some embodiments, a metal-containing material can include one or more transition metal elements (e.g., scandium, titanium, vanadium, chromium, manganese, iron, cobalt, nickel, copper and/or zinc). As an example, a metal-containing material can contain silver and platinum.

Examples of silver-containing materials include silver oxide, colloidal silver, silver nitrate and silver sulfadiazine, silver carbonate, silver acetate, silver lactate, silver citrate, silver hydroxide, silver succinate, silver chlorate, silver stearate, silver sorbate, silver oleate, silver gluconate, silver glycolate, silver adipate, silver myristate, silver benzoate, silver methanesulfonate, silver trifluoracetate, silver trifluoromethanesulfonate, silver behenate, silver phthalate, silver oxalate, silver sulfonate, and alkali silver thiosulphate (e.g., sodium silver thiosulphate, potassium silver thiosulphate).

In addition to one or more metal elements, a metal-containing material can contain, for example, oxygen, nitrogen, carbon, boron, sulfur, phosphorus, silicon, a halogen (e.g., fluorine, chlorine, bromine, iodine) and/or hydrogen. Examples of such metal-containing materials include metal oxides, metal hydroxides, metal nitrides, metal carbides, metal phosphides, metal silicates, metal borides, metal sulfides, metal halides (e.g., metal fluorides, metal chlorides, metal bromides, metal iodides), metal myristates, metal sorbates, metal stearates, metal oleates, metal gluconates, metal glycolates, metal adipates, metal silicates, metal phosphides, metal hydrides, metal nitrates, metal carbonates, metal sulfadiazines, metal hydrides, metal acetates, metal lactates, metal citrates, metal benzoate, metal methanesulfonate, metal trifluoracetate, metal trifluoromethanesulfonate, metal behenate, metal phthalate, metal oxalate, metal sulfonate, alkali metal thiosulphates (e.g., sodium metal thiosulphate, potassium metal thiosulphate). In certain embodiments, a metal-containing material contains at least about one atomic percent (e.g., at least about three atomic percent, at least about five atomic percent, at least about 10 atomic percent, at least about 20 atomic percent, at least about 30 atomic percent, at least about 40 atomic percent, at least about 50 atomic percent) and/or at most about 90 atomic percent (e.g., at most about 80 atomic percent, at most about 70 atomic percent, at most about 60 atomic percent, at most about 50 atomic percent, at most about 40 atomic percent, at most about 30 atomic percent, at most about 20 atomic percent, at most about 15 atomic percent, at most about 12 atomic percent, at most about 10 atomic percent) of nonmetallic elements. For example, in some embodiments, a silver-containing material can contain oxygen in an amount from about five atomic percent to about 20 atomic percent (e.g., from about five atomic percent to about 15 atomic percent, from about eight atomic percent to about 12 atomic percent).

In certain embodiments, the metal-containing materials are an antimicrobial material, an anti-biofilm, an antibacterial material, an anti-inflammatory material, an antifungal material, an antiviral material, an anti-autoimmune material, an anti-cancer material, an MMP modulating material, an atomically disordered crystalline material, and/or a nanocrystalline material.

As used herein, an antimicrobial material herein refers to a material that has sufficient antimicrobial activity to have a beneficial therapeutic effect. In certain embodiments, an antimicrobial material has a corrected zone of inhibition (“CZOI”; defined here as the size of the zone of bacterial growth inhibition corrected so as to not include the size of the antimicrobial test sample creating the zone) of at least about two millimeters (e.g., at least about three millimeters, at least about four millimeters, at least about five millimeters, at least about six millimeters, at least about seven millimeters, at least about eight millimeters, at least about nine millimeters, at least about 10 millimeters). The CZOI of a material is determined as follows. The material is formed as a coating on a dressing (see discussion below), or as a metal-containing dispersion, solution, cream, or gel. The zone of inhibition method is done by streaking bacteria onto Petri dish full of nutrient-containing agar. Bacteria are streaked using a sterile cotton swab, making sure the entire plate is covered to produce a bacterial lawn. Plates are then allowed to dry about 10 min prior to adding test samples. After allowing the bacteria to dry on the plate for about 10 minutes, test samples (e.g. silver coated dressing or devices) are placed onto the Petri dish surface. Plates are then incubated inverted at 32.5° C. for 18-24 hours. Plates are examined following the incubation period by measuring inhibitory zones produced around the test sample. For testing solutions, dispersions, creams, or gels, the standard assay is modified by the creation of 5-mm wells in the agar plates. More than one sample can be tested on each plate providing the wells were placed far enough apart from each other that the zones do not overlap. In most cases 3-4 holes are punched on each plate to the full depth of the agar using a sterilized #3 brass cork bore, creating wells approximately 5 mm in diameter. Bacteria are then streaked onto the plate using a sterile cotton swab, making sure the entire plate is covered to produce a bacterial lawn. Plates are then allowed to dry about 10 min prior to adding test samples. After allowing the bacteria to dry on the plate for about 10 minutes, the silver creams or placebo creams are added using aseptic technique. Creams were put into sterile 3 mL syringes. Each well is filled completely with the corresponding silver cream, approximately 0.1 milliliter. Plates are then incubated inverted at 32.5° C. for 18-24 hours. Plates are examined following the incubation period by measuring inhibitory zones produced around the wells. When testing a solid test material (e.g., a dressing), the zone of inhibition (“ZOI”) is measured and the CZOI is calculated as the ZOI minus the diameter of the test material in contact with the agar. It is to be noted that, while this test for antimicrobial properties is performed on materials that are in the form of a coating on a substrate (e.g., in the form of a dressing), antimicrobial materials are not limited to materials that are coated on a substrate. Rather, a material in any form may be antimicrobial, but it is in the form of a coating on a substrate (e.g., in the form of a dressing) when its antimicrobial properties are tested according to the procedure described herein. When testing a liquid, sol, of gel test material (e.g., a cream), the zone of inhibition (“ZOI”) is measured so as to include the diameter of the well containing the test sample (that is, the ZOI is NOT corrected).

As referred to herein, an atomically disordered, crystalline material (e.g., an atomically disordered, nanocrystalline material) means a material that has more long range ordered, crystalline structure (a lesser degree of defects) than the material has in a fully amorphous state, but that also has less long range, ordered crystalline structure (a higher degree of defects) than the material has in a bulk crystalline state, such as in the form of a cast, wrought or plated material. Examples of defects include point defects, vacancies, line defects, grain boundaries, subgrain boundaries and amorphous regions. Point defects are defects on a size scale of no more than about four atomic spacings. A vacancy is the omission of an atom from its regular atomic site in the crystal lattice. Line defects are defective regions (e.g., edge dislocations, screw dislocations) that result in lattice distortions along a line (which may or may not be a straight line), and generally have a longer scale than point defects. In an edge dislocation, a lattice displacement is produced by a plane of atoms that forms a terminus of the lattice. In a screw dislocation, part of the lattice is displaced with respect to an adjacent part of the lattice. Grain boundaries separate regions having different crystallographic orientation or misorientation (e.g., high angle grain boundaries, low angle grain boundaries, including tilt boundaries and twist boundaries). Subgrain boundaries refer to low angle grain boundaries. An amorphous region is a region that does not exhibit long range, ordered crystalline structure. In certain embodiments, an atomically disordered, crystalline material (e.g., an atomically disordered, nanocrystalline material) has a degree of atomic disorder that is about the same as the degree of atomic disorder of the nanocrystalline silver coating of a member of the Acticoat® family of dressings (Smith & Nephew, Hull, UK) (e.g., an Acticoat® dressing, an Acticoat7® dressing, an Acticoat® moisture coating dressing, an Acticoat® absorbent dressings). In some embodiments, an atomically disordered, crystalline material (e.g., an atomically disordered, nanocrystalline material) has a degree of atomic disorder that is about the same as the degree of atomic disorder of the nanocrystalline silver coatings having a CZOI of at least five millimeters that are disclosed in the examples of Burrell et al., U.S. Pat. No. 5,958,440. In certain embodiments, an atomically disordered, crystalline material (e.g., an atomically disordered, nanocrystalline material), when contacted with an alcohol or water-based electrolyte, is released into the alcohol or water-based electrolyte (e.g., as ions, atoms, molecules and/or clusters) over a time scale of at least about one hour (e.g., at least about two hours, at least about 10 hours, at least about a day). Examples of alcohols and/or water-based electrolytes include body fluids (e.g., blood, urine, saliva) and body tissue (e.g., skin, muscle, bone).

As referred to herein, a nanocrystalline material is a single-phase polycrystal or a multi-phase polycrystal having a maximum dimension of about 100 nanometers or less (e.g., about 90 nanometers or less, about 80 nanometers or less, about 70 nanometers or less, about 60 nanometers or less, about 50 nanometers or less, about 40 nanometers or less, about 30 nanometers or less, about 25 nanometers or less) in at least one dimension.

Examples of antimicrobial metal-containing materials (which may or may not also be an atomically disordered crystalline material or a nanocrystalline material) include antimicrobial silver-containing materials (e.g., antimicrobial silver, antimicrobial silver alloys, antimicrobial silver oxides, antimicrobial silver carbides, antimicrobial silver nitrides, antimicrobial silver borides, antimicrobial silver sulfides, antimicrobial silver myristates, antimicrobial silver stearates, antimicrobial silver oleates, antimicrobial silver gluconates, antimicrobial silver glycolates, antimicrobial silver adipates, antimicrobial silver silicates, antimicrobial silver phosphides, antimicrobial silver halides, antimicrobial silver hydrides, antimicrobial silver nitrates, antimicrobial silver carbonates, antimicrobial silver sulfadiazines, antimicrobial silver acetates, antimicrobial silver lactates, antimicrobial silver citrates, antimicrobial silver benzoate, antimicrobial silver methanesulfonate, antimicrobial silver trifluoracetate, antimicrobial silver trifluoromethanesulfonate, antimicrobial silver behenate, antimicrobial silver phthalate, antimicrobial silver oxalate, antimicrobial silver sulfonate, antimicrobial alkali silver thiosulphates (e.g., antimicrobial sodium silver thiosulphate, antimicrobial potassium silver thiosulphate)), antimicrobial gold-containing materials (e.g., antimicrobial gold, antimicrobial gold alloys, antimicrobial gold oxides, antimicrobial gold carbides, antimicrobial gold nitrides, antimicrobial gold borides, antimicrobial gold sulfides, antimicrobial gold myristates, antimicrobial gold stearates, antimicrobial gold oleates, antimicrobial gold gluconates, antimicrobial gold glycolates, antimicrobial gold adipates, antimicrobial gold silicates, antimicrobial gold phosphides, antimicrobial gold halides, antimicrobial gold hydrides, antimicrobial gold nitrates, antimicrobial gold carbonates, antimicrobial gold sulfadiazines, antimicrobial gold acetates, antimicrobial gold lactates, antimicrobial gold citrates, antimicrobial gold benzoate, antimicrobial gold methanesulfonate, antimicrobial gold trifluoracetate, antimicrobial gold trifluoromethanesulfonate, antimicrobial gold behenate, antimicrobial gold phthalate, antimicrobial gold oxalate, antimicrobial gold sulfonate, antimicrobial alkali gold thiosulphates (e.g., antimicrobial sodium gold thiosulphate, antimicrobial potassium gold thiosulphate)), antimicrobial platinum-containing materials (e.g., antimicrobial platinum, antimicrobial platinum alloys, antimicrobial platinum oxides, antimicrobial platinum carbides, antimicrobial platinum nitrides, antimicrobial platinum borides, antimicrobial platinum sulfides, antimicrobial platinum myristates, antimicrobial platinum stearates, antimicrobial platinum oleates, antimicrobial platinum gluconates, antimicrobial platinum glycolates, antimicrobial platinum adipates, antimicrobial platinum silicates, antimicrobial platinum phosphides, antimicrobial platinum halides, antimicrobial platinum hydrides, antimicrobial platinum nitrates, antimicrobial platinum carbonates, antimicrobial platinum sulfadiazines, antimicrobial platinum acetates, antimicrobial platinum lactates, antimicrobial platinum citrates, antimicrobial platinum benzoate, antimicrobial platinum methanesulfonate, antimicrobial platinum trifluoracetate, antimicrobial platinum trifluoromethanesulfonate, antimicrobial platinum behenate, antimicrobial platinum phthalate, antimicrobial platinum oxalate, antimicrobial platinum sulfonate, antimicrobial alkali platinum thiosulphates (e.g., antimicrobial sodium platinum thiosulphate, antimicrobial potassium platinum thiosulphate)), antimicrobial palladium-containing materials (e.g., antimicrobial palladium, antimicrobial palladium alloys, antimicrobial palladium oxides, antimicrobial palladium carbides, antimicrobial palladium nitrides, antimicrobial palladium borides, antimicrobial palladium sulfides, antimicrobial palladium myristates, antimicrobial palladium stearates, antimicrobial palladium oleates, antimicrobial palladium gluconates, antimicrobial palladium glycolates, antimicrobial palladium adipates, antimicrobial palladium silicates, antimicrobial palladium phosphides, antimicrobial palladium halides, antimicrobial palladium hydrides, antimicrobial palladium nitrates, antimicrobial palladium carbonates, antimicrobial palladium sulfadiazines, antimicrobial palladium acetates, antimicrobial palladium lactates, antimicrobial palladium citrates, antimicrobial palladium benzoate, antimicrobial palladium methanesulfonate, antimicrobial palladium trifluoracetate, antimicrobial palladium trifluoromethanesulfonate, antimicrobial palladium behenate, antimicrobial palladium phthalate, antimicrobial palladium oxalate, antimicrobial palladium sulfonate, antimicrobial alkali palladium thiosulphates (e.g., antimicrobial sodium palladium thiosulphate, antimicrobial potassium palladium thiosulphate)), antimicrobial iridium-containing materials (e.g., antimicrobial iridium, antimicrobial iridium alloys, antimicrobial iridium oxides, antimicrobial iridium carbides, antimicrobial iridium nitrides, antimicrobial iridium borides, antimicrobial iridium sulfides, antimicrobial iridium myristates, antimicrobial iridium stearates, antimicrobial iridium oleates, antimicrobial iridium gluconates, antimicrobial iridium glycolates, antimicrobial iridium adipates, antimicrobial iridium silicates, antimicrobial iridium phosphides, antimicrobial iridium halides, antimicrobial iridium hydrides, antimicrobial iridium nitrates, antimicrobial iridium carbonates, antimicrobial iridium sulfides, antimicrobial iridium sulfadiazines, antimicrobial iridium acetates, antimicrobial iridium lactates, antimicrobial iridium citrates, antimicrobial iridium benzoate, antimicrobial iridium methanesulfonate, antimicrobial iridium trifluoracetate, antimicrobial iridium trifluoromethanesulfonate, antimicrobial iridium behenate, antimicrobial iridium phthalate, antimicrobial iridium oxalate, antimicrobial iridium sulfonate, antimicrobial alkali iridium thiosulphates (e.g., antimicrobial sodium iridium thiosulphate, antimicrobial potassium iridium thiosulphate)), antimicrobial zinc-containing materials (e.g., antimicrobial zinc, antimicrobial zinc alloys, antimicrobial zinc oxides, antimicrobial zinc carbides, antimicrobial zinc nitrides, antimicrobial zinc borides, antimicrobial zinc sulfides, antimicrobial zinc myristates, antimicrobial zinc stearates, antimicrobial zinc oleates, antimicrobial zinc gluconates, antimicrobial zinc glycolates, antimicrobial zinc adipates, antimicrobial zinc silicates, antimicrobial zinc phosphides, antimicrobial zinc halides, antimicrobial zinc hydrides, antimicrobial zinc nitrates, antimicrobial zinc carbonates, antimicrobial zinc sulfides, antimicrobial zinc sulfadiazines, antimicrobial zinc acetates, antimicrobial zinc lactates, antimicrobial zinc citrates, antimicrobial zinc benzoate, antimicrobial zinc methanesulfonate, antimicrobial zinc trifluoracetate, antimicrobial zinc trifluoromethanesulfonate, antimicrobial zinc behenate, antimicrobial zinc phthalate, antimicrobial zinc oxalate, antimicrobial zinc sulfonate), antimicrobial copper-containing materials (e.g., antimicrobial copper, antimicrobial copper alloys, antimicrobial copper oxides, antimicrobial copper carbides, antimicrobial copper nitrides, antimicrobial copper borides, antimicrobial copper sulfides, antimicrobial copper myristates, antimicrobial copper stearates, antimicrobial copper oleates, antimicrobial copper gluconates, antimicrobial copper glycolates, antimicrobial copper adipates, antimicrobial copper silicates, antimicrobial copper phosphides, antimicrobial copper halides, antimicrobial copper hydrides, antimicrobial copper nitrates, antimicrobial copper carbonates, antimicrobial copper sulfides, antimicrobial copper sulfadiazines, antimicrobial copper acetates, antimicrobial copper lactates, antimicrobial copper citrates, antimicrobial copper benzoate, antimicrobial copper methanesulfonate, antimicrobial copper trifluoracetate, antimicrobial copper trifluoromethanesulfonate, antimicrobial copper behenate, antimicrobial copper phthalate, antimicrobial copper oxalate, antimicrobial copper sulfonate, antimicrobial alkali copper thiosulphates (e.g., antimicrobial sodium copper thiosulphate, antimicrobial potassium copper thiosulphate)), antimicrobial tin-containing materials (e.g., antimicrobial tin, antimicrobial tin alloys, antimicrobial tin oxides, antimicrobial tin carbides, antimicrobial tin nitrides, antimicrobial tin borides, antimicrobial tin sulfides, antimicrobial tin myristates, antimicrobial tin stearates, antimicrobial tin oleates, antimicrobial tin gluconates, antimicrobial tin glycolates, antimicrobial tin adipates, antimicrobial tin silicates, antimicrobial tin phosphides, antimicrobial tin halides, antimicrobial tin hydrides, antimicrobial tin nitrates, antimicrobial tin carbonates, antimicrobial tin sulfides, antimicrobial tin sulfadiazines, antimicrobial tin acetates, antimicrobial tin lactates, antimicrobial tin citrates, antimicrobial tin benzoate, antimicrobial tin methanesulfonate, antimicrobial tin trifluoracetate, antimicrobial tin trifluoromethanesulfonate, antimicrobial tin behenate, antimicrobial tin phthalate, antimicrobial tin oxalate, antimicrobial tin sulfonate, antimicrobial alkali tin thiosulphates (e.g., antimicrobial sodium tin thiosulphate, antimicrobial potassium tin thiosulphate)), antimicrobial antimony-containing materials (e.g., antimicrobial antimony, antimicrobial antimony alloys, antimicrobial antimony oxides, antimicrobial antimony carbides, antimicrobial antimony nitrides, antimicrobial antimony borides, antimicrobial antimony sulfides, antimicrobial antimony myristates, antimicrobial antimony stearates, antimicrobial antimony oleates, antimicrobial antimony gluconates, antimicrobial antimony glycolates, antimicrobial antimony adipates, antimicrobial antimony silicates, antimicrobial antimony phosphides, antimicrobial antimony halides, antimicrobial antimony hydrides, antimicrobial antimony nitrates, antimicrobial antimony carbonates, antimicrobial antimony sulfides, antimicrobial antimony sulfadiazines, antimicrobial antimony acetates, antimicrobial antimony lactates, antimicrobial antimony citrates, antimicrobial antimony benzoate, antimicrobial antimony methanesulfonate, antimicrobial antimony trifluoracetate, antimicrobial antimony trifluoromethanesulfonate, antimicrobial antimony behenate, antimicrobial antimony phthalate, antimicrobial antimony oxalate, antimicrobial antimony sulfonate, antimicrobial alkali antimony thiosulphates (e.g., antimicrobial sodium antimony thiosulphate, antimicrobial potassium antimony thiosulphate)), antimicrobial bismuth containing materials (e.g., antimicrobial bismuth, antimicrobial bismuth alloys, antimicrobial bismuth oxides, antimicrobial bismuth carbides, antimicrobial bismuth nitrides, antimicrobial bismuth borides, antimicrobial bismuth sulfides, antimicrobial bismuth myristates, antimicrobial bismuth stearates, antimicrobial bismuth oleates, antimicrobial bismuth gluconates, antimicrobial bismuth glycolates, antimicrobial bismuth adipates, antimicrobial bismuth silicates, antimicrobial bismuth phosphides, antimicrobial bismuth halides, antimicrobial bismuth hydrides, antimicrobial bismuth nitrates, antimicrobial bismuth carbonates, antimicrobial bismuth sulfides, antimicrobial bismuth sulfadiazines, antimicrobial bismuth acetates, antimicrobial bismuth lactates, antimicrobial bismuth citrates, antimicrobial bismuth benzoate, antimicrobial bismuth methanesulfonate, antimicrobial bismuth trifluoracetate, antimicrobial bismuth trifluoromethanesulfonate, antimicrobial bismuth behenate, antimicrobial bismuth phthalate, antimicrobial bismuth oxalate, antimicrobial bismuth sulfonate, antimicrobial alkali bismuth thiosulphates (e.g., antimicrobial sodium bismuth thiosulphate, antimicrobial potassium bismuth thiosulphate)).

While the preceding paragraph lists certain metal-containing materials that are anti-microbial, similar metal-containing materials (oxides, carbides, nitrides, borides, sulfides, myristates, stearates, oleates, gluconates, glycolates, adipates, silicates, phosphides, halides, hydrides, nitrates, hydroxides, carbonates, sulfides, sulfadiazines, acetates, lactates, citrates, benzoates, methanesulfonates, trifluoracetates, trifluoromethanesulfonates, behenates, phthalates, oxalates, sulfonates, and/or alkali metal thiosulphates of silver, gold, palladium, platinum, tin, iridium, antimony, bismuth, copper) can be anti-biofilm materials, antibacterial materials, anti-inflammatory materials, antifungal materials, antiviral materials, anti-autoimmune materials, anti-cancer materials, and/or MMP modulating materials.

Examples of nanocrystalline metal-containing materials (which may or may not also be an antimicrobial material or an atomically disordered crystalline material) include nanocrystalline silver-containing materials (e.g., nanocrystalline silver, nanocrystalline silver alloys, nanocrystalline silver oxides, nanocrystalline silver carbides, nanocrystalline silver nitrides, nanocrystalline silver borides, nanocrystalline silver sulfides, nanocrystalline silver halides, nanocrystalline silver myristates, nanocrystalline silver stearates, nanocrystalline silver oleates, nanocrystalline silver gluconates, nanocrystalline silver glycolates, nanocrystalline silver adipates, nanocrystalline silver silicates, nanocrystalline silver phosphides, nanocrystalline silver hydrides, nanocrystalline silver nitrates, nanocrystalline silver carbonates, nanocrystalline silver sulfides, nanocrystalline silver sulfadiazines, nanocrystalline silver acetates, nanocrystalline silver lactates, nanocrystalline silver citrates, nanocrystalline silver benzoate, nanocrystalline silver methanesulfonate, nanocrystalline silver trifluoracetate, nanocrystalline silver trifluoromethanesulfonate, nanocrystalline silver behenate, nanocrystalline silver phthalate, nanocrystalline silver oxalate, nanocrystalline silver sulfonate, nanocrystalline alkali silver thiosulphates (e.g., nanocrystalline sodium silver thiosulphate, nanocrystalline potassium silver thiosulphate)), nanocrystalline gold-containing materials (e.g., nanocrystalline gold, nanocrystalline gold alloys, nanocrystalline gold oxides, nanocrystalline gold carbides, nanocrystalline gold nitrides, nanocrystalline gold borides, nanocrystalline gold sulfides, nanocrystalline gold halides, nanocrystalline gold hydrides, nanocrystalline gold nitrates, nanocrystalline gold myristates, nanocrystalline gold stearates, nanocrystalline gold oleates, nanocrystalline gold gluconates, nanocrystalline gold glycolates, nanocrystalline gold adipates, nanocrystalline gold silicates, nanocrystalline gold phosphides, nanocrystalline gold carbonates, nanocrystalline gold sulfides, nanocrystalline gold sulfadiazines, nanocrystalline gold acetates, nanocrystalline gold lactates, nanocrystalline gold citrates, nanocrystalline gold benzoate, nanocrystalline gold methanesulfonate, nanocrystalline gold trifluoracetate, nanocrystalline gold trifluoromethanesulfonate, nanocrystalline gold behenate, nanocrystalline gold phthalate, nanocrystalline gold oxalate, nanocrystalline gold sulfonate, nanocrystalline alkali gold thiosulphates (e.g., nanocrystalline sodium gold thiosulphate, nanocrystalline potassium gold thiosulphate)), nanocrystalline platinum-containing materials (e.g., nanocrystalline platinum, nanocrystalline platinum alloys, nanocrystalline platinum oxides, nanocrystalline platinum carbides, nanocrystalline platinum nitrides, nanocrystalline platinum borides, nanocrystalline platinum sulfides, nanocrystalline platinum myristates, nanocrystalline platinum stearates, nanocrystalline platinum oleates, nanocrystalline platinum gluconates, nanocrystalline platinum glycolates, nanocrystalline platinum adipates, nanocrystalline platinum silicates, nanocrystalline platinum phosphides, nanocrystalline platinum halides, nanocrystalline platinum hydrides, nanocrystalline platinum nitrates, nanocrystalline platinum carbonates, nanocrystalline platinum sulfides, nanocrystalline platinum sulfadiazines, nanocrystalline platinum acetates, nanocrystalline platinum lactates, nanocrystalline platinum citrates, nanocrystalline platinum benzoate, nanocrystalline platinum methanesulfonate, nanocrystalline platinum trifluoracetate, nanocrystalline platinum trifluoromethanesulfonate, nanocrystalline platinum behenate, nanocrystalline platinum phthalate, nanocrystalline platinum oxalate, nanocrystalline platinum sulfonate, nanocrystalline alkali platinum thiosulphates (e.g., nanocrystalline sodium platinum thiosulphate, nanocrystalline potassium platinum thiosulphate)), nanocrystalline palladium-containing materials (e.g., nanocrystalline palladium, nanocrystalline palladium alloys, nanocrystalline palladium oxides, nanocrystalline palladium carbides, nanocrystalline palladium nitrides, nanocrystalline palladium borides, nanocrystalline palladium sulfides, nanocrystalline palladium myristates, nanocrystalline palladium stearates, nanocrystalline palladium oleates, nanocrystalline palladium glycolates, nanocrystalline palladium gluconates, nanocrystalline palladium adipates, nanocrystalline palladium silicates, nanocrystalline palladium phosphides, nanocrystalline palladium halides, nanocrystalline palladium hydrides, nanocrystalline palladium nitrates, nanocrystalline palladium carbonates, nanocrystalline palladium sulfides, nanocrystalline palladium sulfadiazines, nanocrystalline palladium acetates, nanocrystalline palladium lactates, nanocrystalline palladium citrates, nanocrystalline palladium benzoate, nanocrystalline palladium methanesulfonate, nanocrystalline palladium trifluoracetate, nanocrystalline palladium trifluoromethanesulfonate, nanocrystalline palladium behenate, nanocrystalline palladium phthalate, nanocrystalline palladium oxalate, nanocrystalline palladium sulfonate, nanocrystalline alkali palladium thiosulphates (e.g., nanocrystalline sodium palladium thiosulphate, nanocrystalline potassium palladium thiosulphate)), nanocrystalline iridium-containing materials (e.g., nanocrystalline iridium, nanocrystalline iridium alloys, nanocrystalline iridium oxides, nanocrystalline iridium carbides, nanocrystalline iridium nitrides, nanocrystalline iridium borides, nanocrystalline iridium sulfides, nanocrystalline iridium myristates, nanocrystalline iridium stearates, nanocrystalline iridium oleates, nanocrystalline iridium gluconates, nanocrystalline iridium glycolates, nanocrystalline iridium adipates, nanocrystalline iridium silicates, nanocrystalline iridium phosphides, nanocrystalline iridium halides, nanocrystalline iridium hydrides, nanocrystalline iridium nitrates, nanocrystalline iridium carbonates, nanocrystalline iridium sulfides, nanocrystalline iridium sulfadiazines, nanocrystalline iridium acetates, nanocrystalline iridium lactates, nanocrystalline iridium citrates, nanocrystalline iridium benzoate, nanocrystalline iridium methanesulfonate, nanocrystalline iridium trifluoracetate, nanocrystalline iridium trifluoromethanesulfonate, nanocrystalline iridium behenate, nanocrystalline iridium phthalate, nanocrystalline iridium oxalate, nanocrystalline iridium sulfonate, nanocrystalline alkali iridium thiosulphates (e.g., nanocrystalline sodium iridium thiosulphate, nanocrystalline potassium iridium thiosulphate)), nanocrystalline zinc-containing materials (e.g., nanocrystalline zinc, nanocrystalline zinc alloys, nanocrystalline zinc oxides, nanocrystalline zinc carbides, nanocrystalline zinc nitrides, nanocrystalline zinc borides, nanocrystalline zinc sulfides, nanocrystalline zinc myristates, nanocrystalline zinc stearates, nanocrystalline zinc oleates, nanocrystalline zinc gluconates, nanocrystalline zinc glycolates, nanocrystalline zinc adipates, nanocrystalline zinc silicates, nanocrystalline zinc phosphides, nanocrystalline zinc halides, nanocrystalline zinc hydrides, nanocrystalline zinc nitrates, nanocrystalline zinc carbonates, nanocrystalline zinc sulfides, nanocrystalline zinc sulfadiazines, nanocrystalline zinc acetates, nanocrystalline zinc lactates, nanocrystalline zinc citrates, nanocrystalline zinc benzoate, nanocrystalline zinc methanesulfonate, nanocrystalline zinc trifluoracetate, nanocrystalline zinc trifluoromethanesulfonate, nanocrystalline zinc behenate, nanocrystalline zinc phthalate, nanocrystalline zinc oxalate, nanocrystalline zinc sulfonate), nanocrystalline copper-containing materials (e.g., nanocrystalline copper, nanocrystalline copper alloys, nanocrystalline copper oxides, nanocrystalline copper carbides, nanocrystalline copper nitrides, nanocrystalline copper borides, nanocrystalline copper sulfides, nanocrystalline copper myristates, nanocrystalline copper stearates, nanocrystalline copper oleates, nanocrystalline copper gluconates, nanocrystalline copper glycolates, nanocrystalline copper adipates, nanocrystalline copper silicates, nanocrystalline copper phosphides, nanocrystalline copper halides, nanocrystalline copper hydrides, nanocrystalline copper nitrates, nanocrystalline copper carbonates, nanocrystalline copper sulfadiazines, nanocrystalline copper acetates, nanocrystalline copper lactates, nanocrystalline copper citrates, nanocrystalline copper benzoate, nanocrystalline copper methanesulfonate, nanocrystalline copper trifluoracetate, nanocrystalline copper trifluoromethanesulfonate, nanocrystalline copper behenate, nanocrystalline copper phthalate, nanocrystalline copper oxalate, nanocrystalline copper sulfonate, nanocrystalline alkali copper thiosulphates (e.g., nanocrystalline sodium copper thiosulphate, nanocrystalline potassium copper thiosulphate)), nanocrystalline tin-containing materials (e.g., nanocrystalline tin, nanocrystalline tin alloys, nanocrystalline tin oxides, nanocrystalline tin carbides, nanocrystalline tin nitrides, nanocrystalline tin borides, nanocrystalline tin sulfides, nanocrystalline tin myristates, nanocrystalline tin stearates, nanocrystalline tin oleates, nanocrystalline tin glycolates, nanocrystalline tin gluconates, nanocrystalline tin adipates, nanocrystalline tin silicates, nanocrystalline tin phosphides, nanocrystalline tin halides, nanocrystalline tin hydrides, nanocrystalline tin nitrates, nanocrystalline tin carbonates, nanocrystalline tin sulfides, nanocrystalline tin sulfadiazines, nanocrystalline tin acetates, nanocrystalline tin lactates, nanocrystalline tin citrates, nanocrystalline tin benzoate, nanocrystalline tin methanesulfonate, nanocrystalline tin trifluoracetate, nanocrystalline tin trifluoromethanesulfonate, nanocrystalline tin behenate, nanocrystalline tin phthalate, nanocrystalline tin oxalate, nanocrystalline tin sulfonate, nanocrystalline alkali tin thiosulphates (e.g., nanocrystalline sodium tin thiosulphate, nanocrystalline potassium tin thiosulphate)), nanocrystalline antimony-containing materials (e.g., nanocrystalline antimony, nanocrystalline antimony alloys, nanocrystalline antimony oxides, nanocrystalline antimony carbides, nanocrystalline antimony nitrides, nanocrystalline antimony borides, nanocrystalline antimony sulfides, nanocrystalline antimony myristates, nanocrystalline antimony stearates, nanocrystalline antimony oleates, nanocrystalline antimony gluconates, nanocrystalline antimony glycolates, nanocrystalline antimony adipates, nanocrystalline antimony silicates, nanocrystalline antimony phosphides, nanocrystalline antimony halides, nanocrystalline antimony hydrides, nanocrystalline antimony nitrates, nanocrystalline antimony carbonates, nanocrystalline antimony sulfides, nanocrystalline antimony sulfadiazines, nanocrystalline antimony acetates, nanocrystalline antimony lactates, nanocrystalline antimony citrates, nanocrystalline antimony benzoate, nanocrystalline antimony methanesulfonate, nanocrystalline antimony trifluoracetate, nanocrystalline antimony trifluoromethanesulfonate, nanocrystalline antimony behenate, nanocrystalline antimony phthalate, nanocrystalline antimony oxalate, nanocrystalline antimony sulfonate, nanocrystalline alkali antimony thiosulphates (e.g., nanocrystalline sodium antimony thiosulphate, nanocrystalline potassium antimony thiosulphate)), nanocrystalline bismuth containing materials (e.g., nanocrystalline bismuth, nanocrystalline bismuth alloys, nanocrystalline bismuth oxides, nanocrystalline bismuth carbides, nanocrystalline bismuth nitrides, nanocrystalline bismuth borides, nanocrystalline bismuth sulfides, nanocrystalline bismuth myristates, nanocrystalline bismuth stearates, nanocrystalline bismuth oleates, nanocrystalline bismuth gluconates, nanocrystalline bismuth glycolates, nanocrystalline bismuth adipates, nanocrystalline bismuth silicates, nanocrystalline bismuth phosphides, nanocrystalline bismuth halides, nanocrystalline bismuth hydrides, nanocrystalline bismuth nitrates, nanocrystalline bismuth carbonates, nanocrystalline bismuth sulfides, nanocrystalline anti bismuth sulfadiazines, nanocrystalline bismuth acetates, nanocrystalline bismuth lactates, nanocrystalline bismuth citrates, nanocrystalline bismuth benzoate, nanocrystalline bismuth methanesulfonate, nanocrystalline bismuth trifluoracetate, nanocrystalline bismuth trifluoromethanesulfonate, nanocrystalline bismuth behenate, nanocrystalline bismuth phthalate, nanocrystalline bismuth oxalate, nanocrystalline bismuth sulfonate, nanocrystalline alkali bismuth thiosulphates (e.g., nanocrystalline sodium bismuth thiosulphate, nanocrystalline potassium bismuth thiosulphate)).

Examples of atomically disordered, crystalline metal-containing material (which may or may not also be an antimicrobial material or a nanocrystalline material) include atomically disordered, crystalline silver-containing materials (e.g., atomically disordered, crystalline silver; atomically disordered, crystalline silver alloys; atomically disordered, crystalline silver oxides; atomically disordered, crystalline silver carbides; atomically disordered, crystalline silver nitrides; atomically disordered, crystalline silver borides; atomically disordered, crystalline silver sulfides; atomically disordered, crystalline silver myristates; atomically disordered, crystalline silver stearates; atomically disordered, crystalline silver oleates; atomically disordered, crystalline silver gluconates; atomically disordered, crystalline silver glycolates; atomically disordered, crystalline silver adipates; atomically disordered, crystalline silver silicates; atomically disordered, crystalline silver phosphides; atomically disordered, crystalline silver halides; atomically disordered, crystalline silver hydrides; atomically, crystalline silver nitrates; atomically disordered, crystalline silver carbonates; atomically disordered, crystalline silver sulfides; atomically disordered, crystalline silver sulfadiazines; atomically disordered, crystalline silver acetates; atomically disordered, crystalline silver lactates; atomically disordered, crystalline silver citrates; atomically disordered, crystalline silver benzoate; atomically disordered, crystalline silver methanesulfonate; atomically disordered, crystalline silver trifluoracetate; atomically disordered, crystalline silver trifluoromethanesulfonate; atomically disordered, crystalline silver behenate; atomically disordered, crystalline silver phthalate; atomically disordered, crystalline silver oxalate; atomically disordered, crystalline silver sulfonate; atomically disordered, crystalline alkali silver thiosulphates (e.g., atomically disordered, crystalline sodium silver thiosulphate, atomically disordered, crystalline potassium silver thiosulphate)), atomically disordered, crystalline gold-containing materials (atomically disordered, crystalline gold; atomically disordered, crystalline gold alloys; atomically disordered, crystalline gold oxides; atomically disordered, crystalline gold carbides; atomically disordered, crystalline gold nitrides; atomically disordered, crystalline gold borides; atomically disordered, crystalline gold sulfides; atomically disordered, crystalline gold myristates; atomically disordered, crystalline gold stearates; atomically disordered, crystalline gold oleates; atomically disordered, crystalline gold gluconates; atomically disordered, crystalline gold glycolates; atomically disordered, crystalline gold adipates; atomically disordered, crystalline gold silicates; atomically disordered, crystalline gold phosphides; atomically disordered, crystalline gold halides; atomically disordered, crystalline gold hydrides, atomically disordered, crystalline gold nitrates; atomically disordered, crystalline gold carbonates; atomically disordered, crystalline gold sulfides; atomically disordered, crystalline gold sulfadiazines; atomically disordered, crystalline gold acetates; atomically disordered, crystalline gold lactates; atomically disordered, crystalline gold citrates; atomically disordered, crystalline gold benzoate; atomically disordered, crystalline gold methanesulfonate; atomically disordered, crystalline gold trifluoracetate; atomically disordered, crystalline gold trifluoromethanesulfonate; atomically disordered, crystalline gold behenate; atomically disordered, crystalline gold phthalate; atomically disordered, crystalline gold oxalate; atomically disordered, crystalline gold sulfonate; atomically disordered, crystalline alkali gold thiosulphates (e.g., atomically disordered, crystalline sodium gold thiosulphate, atomically disordered, crystalline potassium gold thiosulphate)), atomically disordered, crystalline platinum-containing materials (e.g., atomically disordered, crystalline platinum; atomically disordered, crystalline platinum alloys; atomically disordered, crystalline platinum oxides; atomically disordered, crystalline platinum carbides; atomically disordered, crystalline platinum nitrides; atomically disordered, crystalline platinum borides; atomically disordered, crystalline platinum sulfides; atomically disordered, crystalline platinum myristates; atomically disordered, crystalline platinum stearates; atomically disordered, crystalline platinum oleates; atomically disordered, crystalline platinum gluconates; atomically disordered, crystalline platinum glycolates; atomically disordered, crystalline platinum adipates; atomically disordered, crystalline platinum silicates; atomically disordered, crystalline platinum phosphides; atomically disordered, crystalline platinum halides; atomically disordered, crystalline platinum hydrides, atomically disordered, crystalline platinum nitrates; atomically disordered, crystalline platinum carbonates; atomically disordered, crystalline platinum sulfides; atomically disordered, crystalline platinum sulfadiazines; atomically disordered, crystalline platinum acetates; atomically disordered, crystalline platinum lactates; atomically disordered, crystalline platinum citrates; atomically disordered, crystalline platinum benzoate; atomically disordered, crystalline platinum methanesulfonate; atomically disordered, crystalline platinum trifluoracetate; atomically disordered, crystalline platinum trifluoromethanesulfonate; atomically disordered, crystalline platinum behenate; atomically disordered, crystalline platinum phthalate; atomically disordered, crystalline platinum oxalate; atomically disordered, crystalline platinum sulfonate; atomically disordered, crystalline alkali platinum thiosulphates (e.g., atomically disordered, crystalline sodium platinum thiosulphate, atomically disordered, crystalline potassium platinum thiosulphate), atomically disordered, crystalline palladium-containing materials (e.g., atomically disordered, crystalline palladium; atomically disordered, crystalline palladium alloys; atomically disordered, crystalline palladium oxides; atomically disordered, crystalline palladium carbides; atomically disordered, crystalline palladium nitrides; atomically disordered, crystalline palladium borides; atomically disordered, crystalline palladium sulfides; atomically disordered, crystalline palladium myristates; atomically disordered, crystalline palladium stearates; atomically disordered, crystalline palladium oleates; atomically disordered, crystalline palladium gluconates; atomically disordered, crystalline palladium glycolates; atomically disordered, crystalline palladium adipates; atomically disordered, crystalline palladium silicates; atomically disordered, crystalline palladium phosphides; atomically disordered, crystalline palladium halides; atomically disordered, crystalline palladium hydrides, atomically disordered, crystalline palladium nitrates; atomically disordered, crystalline palladium carbonates; atomically disordered, crystalline palladium sulfides; atomically disordered, crystalline palladium sulfadiazines; atomically disordered, crystalline palladium acetates; atomically disordered, crystalline palladium lactates; atomically disordered, crystalline palladium citrates; atomically disordered, crystalline palladium benzoate; atomically disordered, crystalline palladium methanesulfonate; atomically disordered, crystalline palladium trifluoracetate; atomically disordered, crystalline palladium trifluoromethanesulfonate; atomically disordered, crystalline palladium behenate; atomically disordered, crystalline palladium phthalate; atomically disordered, crystalline palladium oxalate; atomically disordered, crystalline palladium sulfonate; atomically disordered, crystalline alkali palladium thiosulphates (e.g., atomically disordered, crystalline sodium palladium thiosulphate, atomically disordered, crystalline potassium palladium thiosulphate)), atomically disordered, crystalline iridium-containing materials (e.g., atomically disordered, crystalline iridium; atomically disordered, crystalline iridium alloys; atomically disordered, crystalline iridium oxides; atomically disordered, crystalline iridium carbides; atomically disordered, crystalline iridium nitrides; atomically disordered, crystalline iridium borides; atomically disordered, crystalline iridium sulfides; atomically disordered, crystalline iridium myristates; atomically disordered, crystalline iridium stearates; atomically disordered, crystalline iridium oleates; atomically disordered, crystalline iridium gluconates; atomically disordered, crystalline iridium glycolates; atomically disordered, crystalline iridium adipates; atomically disordered, crystalline iridium silicates; atomically disordered, crystalline iridium phosphides; atomically disordered, crystalline iridium halides; atomically disordered, crystalline iridium hydrides, atomically disordered, crystalline iridium nitrates; atomically disordered, crystalline iridium carbonates; atomically disordered, crystalline iridium sulfides; atomically disordered, crystalline iridium sulfadiazines; atomically disordered, crystalline iridium acetates; atomically disordered, crystalline iridium lactates; atomically disordered, crystalline iridium citrates; atomically disordered, crystalline iridium benzoate; atomically disordered, crystalline iridium methanesulfonate; atomically disordered, crystalline iridium trifluoracetate; atomically disordered, crystalline iridium trifluoromethanesulfonate; atomically disordered, crystalline iridium behenate; atomically disordered, crystalline iridium phthalate; atomically disordered, crystalline iridium oxalate; atomically disordered, crystalline iridium sulfonate; atomically disordered, crystalline alkali iridium thiosulphates (e.g., atomically disordered, crystalline sodium iridium thiosulphate, atomically disordered, crystalline potassium iridium thiosulphate)), atomically disordered, crystalline zinc-containing materials (e.g., atomically disordered, crystalline zinc; atomically disordered, crystalline zinc alloys; atomically disordered, crystalline zinc oxides; atomically disordered, crystalline zinc carbides; atomically disordered, crystalline zinc nitrides; atomically disordered, crystalline zinc borides; atomically disordered, crystalline zinc sulfides; atomically disordered, crystalline zinc myristates; atomically disordered, crystalline zinc stearates; atomically disordered, crystalline zinc oleates; atomically disordered, crystalline zinc gluconates; atomically disordered, crystalline zinc glycolates; atomically disordered, crystalline zinc adipates; atomically disordered, crystalline zinc silicates; atomically disordered, crystalline zinc phosphides; atomically disordered, crystalline zinc halides; atomically disordered, crystalline zinc hydrides, atomically disordered, crystalline zinc nitrates; atomically disordered, crystalline zinc carbonates; atomically disordered, crystalline zinc sulfides; atomically disordered, crystalline zinc sulfadiazines; atomically disordered, crystalline zinc acetates; atomically disordered, crystalline zinc lactates; atomically disordered, crystalline zinc citrates; atomically disordered, crystalline zinc benzoate; atomically disordered, crystalline zinc methanesulfonate; atomically disordered, crystalline zinc trifluoracetate; atomically disordered, crystalline zinc trifluoromethanesulfonate; atomically disordered, crystalline zinc behenate; atomically disordered, crystalline zinc phthalate; atomically disordered, crystalline zinc oxalate; atomically disordered, crystalline zinc sulfonate), atomically disordered, crystalline copper-containing materials (e.g., atomically disordered, crystalline copper; atomically disordered, crystalline copper alloys; atomically disordered, crystalline copper oxides; atomically disordered, crystalline copper carbides; atomically disordered, crystalline copper nitrides; atomically disordered, crystalline copper borides; atomically disordered, crystalline copper sulfides; atomically disordered, crystalline copper myristates; atomically disordered, crystalline copper stearates; atomically disordered, crystalline copper oleates; atomically disordered, crystalline copper gluconates; atomically disordered, crystalline copper glycolates; atomically disordered, crystalline copper adipates; atomically disordered, crystalline copper silicates; atomically disordered, crystalline copper phosphides; atomically disordered, crystalline copper halides; atomically disordered, crystalline copper hydrides, atomically disordered, crystalline copper nitrates; atomically disordered, crystalline copper carbonates; atomically disordered, crystalline copper sulfides; atomically disordered, crystalline copper sulfadiazines; atomically disordered, crystalline copper acetates; atomically disordered, crystalline copper lactates; atomically disordered, crystalline copper citrates; atomically disordered, crystalline copper benzoate; atomically disordered, crystalline copper methanesulfonate; atomically disordered, crystalline copper trifluoracetate; atomically disordered, crystalline copper trifluoromethanesulfonate; atomically disordered, crystalline copper behenate; atomically disordered, crystalline copper phthalate; atomically disordered, crystalline copper oxalate; atomically disordered, crystalline copper sulfonate; atomically disordered, crystalline copper thiosulphates (e.g., atomically disordered, crystalline sodium copper thiosulphate, atomically disordered, crystalline potassium copper thiosulphate)), atomically disordered, crystalline tin-containing materials (e.g., atomically disordered, crystalline tin; atomically disordered, crystalline tin alloys; atomically disordered, crystalline tin oxides; atomically disordered, crystalline tin carbides; atomically disordered, crystalline tin nitrides; atomically disordered, crystalline tin borides; atomically disordered, crystalline tin sulfides; atomically disordered, crystalline tin myristates; atomically disordered, crystalline tin stearates; atomically disordered, crystalline tin oleates; atomically disordered, crystalline tin gluconates; atomically disordered, crystalline tin glycolates; atomically disordered, crystalline tin adipates; atomically disordered, crystalline tin silicates; atomically disordered, crystalline tin phosphides; atomically disordered, crystalline tin halides; atomically disordered, crystalline tin hydrides, atomically disordered, crystalline tin nitrates; atomically disordered, crystalline tin carbonates; atomically disordered, crystalline tin sulfides; atomically disordered, crystalline tin sulfadiazines; atomically disordered, crystalline tin acetates; atomically disordered, crystalline tin lactates; atomically disordered, crystalline tin citrates; atomically disordered, crystalline tin benzoate; atomically disordered, crystalline tin methanesulfonate; atomically disordered, crystalline tin trifluoracetate; atomically disordered, crystalline tin trifluoromethanesulfonate; atomically disordered, crystalline tin behenate; atomically disordered, crystalline tin phthalate; atomically disordered, crystalline tin oxalate; atomically disordered, crystalline tin sulfonate; atomically disordered, crystalline alkali tin thiosulphates (e.g., atomically disordered, crystalline sodium tin thiosulphate, atomically disordered, crystalline potassium tin thiosulphate)), atomically disordered, crystalline antimony-containing materials (e.g., atomically disordered, crystalline antimony; atomically disordered, crystalline antimony alloys; atomically disordered, crystalline antimony oxides; atomically disordered, crystalline antimony carbides; atomically disordered, crystalline antimony nitrides; atomically disordered, crystalline antimony borides; atomically disordered, crystalline antimony sulfides; atomically disordered, crystalline antimony myristates; atomically disordered, crystalline antimony stearates; atomically disordered, crystalline antimony oleates; atomically disordered, crystalline antimony gluconates; atomically disordered, crystalline antimony glycolates; atomically disordered, crystalline antimony adipates; atomically disordered, crystalline antimony silicates; atomically disordered, crystalline antimony phosphides; atomically disordered, crystalline antimony halides; atomically disordered, crystalline antimony hydrides, atomically disordered, crystalline antimony nitrates; atomically disordered, crystalline antimony carbonates; atomically disordered, crystalline antimony sulfides; atomically disordered, crystalline antimony sulfadiazines; atomically disordered, crystalline antimony acetates; atomically disordered, crystalline antimony lactates; atomically disordered, crystalline antimony citrates; atomically disordered, crystalline antimony benzoate; atomically disordered, crystalline antimony methanesulfonate; atomically disordered, crystalline antimony trifluoracetate; atomically disordered, crystalline antimony trifluoromethanesulfonate; atomically disordered, crystalline antimony behenate; atomically disordered, crystalline antimony phthalate; atomically disordered, crystalline antimony oxalate; atomically disordered, crystalline antimony sulfonate; atomically disordered, crystalline alkali antimony thiosulphates (e.g., atomically disordered, crystalline sodium antimony thiosulphate, atomically disordered, crystalline potassium antimony thiosulphate)), atomically disordered, crystalline bismuth-containing materials (e.g., atomically disordered, crystalline bismuth; atomically disordered, crystalline bismuth alloys; atomically disordered, crystalline bismuth oxides; atomically disordered, crystalline bismuth carbides; atomically disordered, crystalline bismuth nitrides; atomically disordered, crystalline bismuth borides; atomically disordered, crystalline bismuth sulfides; atomically disordered, crystalline bismuth myristates; atomically disordered, crystalline bismuth stearates; atomically disordered, crystalline bismuth oleates; atomically disordered, crystalline bismuth gluconates; atomically disordered, crystalline bismuth glycolates; atomically disordered, crystalline bismuth adipates; atomically disordered, crystalline bismuth silicates; atomically disordered, crystalline bismuth phosphides; atomically disordered, crystalline bismuth halides; atomically disordered, crystalline bismuth hydrides, atomically disordered, crystalline bismuth nitrates; atomically disordered, crystalline bismuth carbonates; atomically disordered, crystalline bismuth sulfides; atomically disordered, crystalline bismuth sulfadiazines; atomically disordered, crystalline bismuth acetates; atomically disordered, crystalline bismuth lactates; atomically disordered, crystalline bismuth citrates; atomically disordered, crystalline bismuth benzoate; atomically disordered, crystalline bismuth methanesulfonate; atomically disordered, crystalline bismuth trifluoracetate; atomically disordered, crystalline bismuth trifluoromethanesulfonate; atomically disordered, crystalline bismuth behenate; atomically disordered, crystalline bismuth phthalate; atomically disordered, crystalline bismuth oxalate; atomically disordered, crystalline bismuth sulfonate; atomically disordered, crystalline alkali bismuth thiosulphates (e.g., atomically disordered, crystalline sodium bismuth thiosulphate, atomically disordered, crystalline potassium bismuth thiosulphate)).

The metal-containing material can be used to treat, for example a human or an animal (e.g., a dog, a cat, a horse, a bird, a reptile, an amphibian, a fish, a turtle, a guinea pig, a hamster, a rodent, a cow, a pig, a goat, a primate, a monkey, a chicken, a turkey, a buffalo, an ostrich, a sheep, a llama).

Substrate Coatings

Examples of commercially available metal-containing materials include the Acticoat® family of dressings (Smith & Nephew, Hull, UK), which are formed of antimicrobial, anti-inflammatory atomically disordered, nanocrystalline silver-containing material coated on one or more substrates. Such dressings include the Acticoat® dressings, the Acticoat7 dressings, the Acticoat® moisture coating dressings, and the Acticoat® absorbent dressings.

A coating of a metal-containing material (e.g., an antimicrobial, atomically disordered, nanocrystalline silver-containing material) can be formed on a substrate using a desired technique. In certain embodiments, the coating is formed by depositing the material on the substrate surface using chemical vapor deposition, physical vapor deposition, and/or liquid phase deposition. Exemplary deposition methods include vacuum evaporation deposition, arc evaporation deposition, reactive sputtering deposition, sputter deposition, magnetron sputter deposition and ion plating.

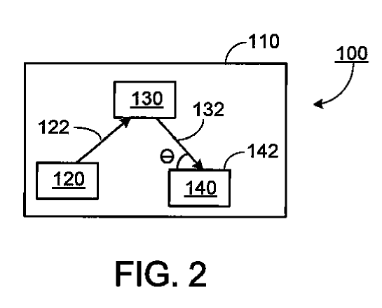

In some embodiments, the coating is prepared using physical vapor deposition. FIG. 2 shows a vapor deposition system 100 that includes a vacuum chamber 110, an energy source 120 (e.g., an electron beam source, an ion source, a laser beam, a magnetron source), a target 130 and a substrate 140. During operation, energy source 120 directs a beam of energy 122 to target 130, causing material 132 to be removed (e.g., by evaporation) from target 130 and directed to a surface 142 of substrate 140. At least a portion of the removed material 132 is deposited on surface 142.

In general, the values of the system parameters (e.g., the temperature of surface 142, the pressure of chamber 110, the angle of incidence of removed material 132 on surface 142, the distance between target 130 and surface 142) can be selected as desired. The temperature of surface 142 can be relatively low during the deposition process. For example, during the deposition process, the ratio of the temperature of substrate 140 to the melting point of the material forming target 130 (as determined in using Kelvin) can be about 0.5 or less (e.g., about 0.4 or less, about 0.35 or less, about 0.3 or less).

The pressure in chamber 110 can be relatively high. For example, vacuum evaporation deposition, electron beam deposition or arc evaporation, the pressure can be about 0.01 milliTorr or greater. For gas scattering evaporation (pressure plating) or reactive arc evaporation, the pressure in chamber 110 can be about 20 milliTorr or greater. For sputter deposition, the pressure in chamber 110 can be about 75 milliTorr or greater. For magnetron sputter deposition, the pressure in chamber 110 can be about 10 milliTorr or greater. For ion plating, the pressure in chamber 110 can be 200 milliTorr or greater.

The angle of incidence of removed material 132 on surface 142 (θ) can be relatively low. For example, the angle of incidence of removed material 132 on surface 142 can be about 75° or less (e.g., about 60° or less, about 45° or less, about 30° or less).

The distance between target 130 and surface 142 can be selected based upon the values of the other system parameters. For example, the distance between target 130 and surface 142 can be about 250 millimeters or less (e.g., about 150 millimeters or less, 125 millimeters or less, about 100 millimeters or less, about 90 millimeters or less, about 80 millimeters or less, about 70 millimeters or less, about 60 millimeters or less, about 50 millimeters or less, about 40 millimeters or less).

As noted above, it is believed that, the metal-containing material, when contacted with an alcohol or water-based electrolyte, can be released into the alcohol or water-based electrolyte (e.g., as ions, atoms, molecules and/or clusters). It is also believed that the ability to release the metal (e.g., as atoms, ions, molecules and/or clusters) on a sustainable basis from a coating is generally dependent upon a number of factors, including coating characteristics such as composition, structure, solubility and thickness, and the nature of the environment in which the device is used. As the level of atomic disorder is increased, it is believed that the amount of metal species released per unit time increases. For example, a silver metal film deposited by magnetron sputtering at a ratio of substrate temperature to the target melting point of less than about 0.5 and a working gas pressure of about 0.93 Pascals (about seven milliTorr) releases approximately ⅓ of the silver ions that a film deposited under similar conditions, but at four Pascals (about 30 milliTorr), will release over 10 days. Coatings formed with an intermediate structure (e.g., lower pressure, lower angle of incidence etc.) have been observed to have metal (e.g., silver) release values intermediate to these values as determined by bioassays. In general, to obtain relatively slow release of the metal, the coating should have a relatively low degree of atomic disorder, and, to obtain relatively fast release of the metal, the coating should have a relatively high degree of atomic disorder.

For continuous, uniform coatings, the time for total dissolution is generally a function of coating thickness and the nature of the environment to which the coating is exposed. The release of metal is believed to increase approximately linearly as the thickness of the coating is increased. For example, it has been observed that a two fold increase in coating thickness can result in about a two fold increase in longevity.

In certain embodiments, it is possible to manipulate the degree of atomic disorder, and therefore the metal release from a coating, by forming a thin film coating with a modulated structure. For example, a coating deposited by magnetron sputtering such that the working gas pressure was relatively low (e.g., about two Pascals or about 15 milliTorr) for about 50% of the deposition time and relatively high (e.g., about four Pascals or 30 milliTorr) for the remaining time, can result in a relatively rapid initial release of metal (e.g., ions, clusters, atoms, molecules), followed by a longer period of slow release. This type of coating is can be particularly effective on devices such as urinary catheters for which an initial rapid release is advantageous to achieve quick antimicrobial concentrations followed by a lower release rate to sustain the concentration of metal (e.g., ions, clusters, atoms, molecules) over a period of weeks.

It is further believed that the degree of atomic disorder of a coating can be manipulated by introducing one or more dissimilar materials into the coating. For example, one or more gases can be present in chamber 110 during the deposition process. Examples of such gases include oxygen-containing gases (e.g., oxygen, air, water), nitrogen-containing gases (e.g., nitrogen, air), hydrogen-containing gases (e.g., water, hydrogen), boron-containing gases (e.g., boron), sulfur-containing gases (e.g., sulfur), carbon-containing gases (e.g., carbon monoxide, carbon dioxide), phosphorus-containing gases, silicon-containing gases, and halogen-containing gases (e.g., fluorine, chlorine, bromine, iodine). The additional gas(es) can be co-deposited or reactively deposited with material 132. This can result in the deposition/formation of an oxide, hydroxide, nitride, carbide, phosphide, silicate, boride, sulfide, hydride, nitrate, carbonate, alkali thiosulphate (e.g., sodium thiosulphate, potassium thiosulphate), myristate, sorbate, stearate, oleate, gluconate, glycolate, adipate, silicate, phosphide, sulfadiazine, acetate, lactate, citrate, benzoate, methanesulfonate, trifluoracetate, trifluoromethanesulfonate, behenate, phthalate, oxalate, sulfonate, and/or halide material (e.g., an oxide of a metal-containing material, a hydroxide of a metal-containing material, a nitride of a metal-containing material, a carbide of a metal-containing material, a phosphide of a metal-containing material, a silicate of a metal-containing material, a boride of a metal-containing material, a sulfide of a metal-containing material, a hydride of a metal-containing material, a halide of a metal-containing material, a nitrate of a metal-containing material, a carbonate of a metal-containing material, a myristate of a metal-containing material, a sorbate of a metal-containing material, a stearate of a metal-containing material, an oleate of a metal-containing material, a gluconate of a metal-containing material, a glycolate of a metal-containing material, an adipate of a metal-containing material, a silicate of a metal-containing material, a phosphide of a metal-containing material, a sulfide of a metal-containing material, a sulfadiazine of a metal-containing material, a sulfadiazine of a metal-containing material, an acetate of a metal-containing material, a lactate of a metal-containing material, a citrate of a metal-containing material, a benzoate of a metal-containing material, a methanesulfonate of a metal-containing material, a trifluoracetate of a metal-containing material, a trifluoromethanesulfonate of a metal-containing material, a behenate of a metal-containing material, a phthalate of a metal-containing material, a oxalate of a metal-containing material, a sulfonate of a metal-containing material, an alkali metal thiosulphate (e.g., sodium metal thiosulphate, potassium metal thiosulphate) of a metal-containing material). Without wishing to be bound by theory, it is believed that atoms and/or molecules of the additional gas(es) may become absorbed or trapped in the material, resulting in enhanced atomic disorder. The additional gas(es) may be continuously supplied during deposition, or may be pulsed to (e.g., for sequential deposition). In embodiments, the material formed can be constituted of a material with a ratio of material 132 to additional gas(es) of about 0.2 or greater. The presence of dissimilar atoms or molecules in the coating can enhance the degree of atomic disorder of the coating due to the difference in atomic radii of the dissimilar constituents in the coating. In some embodiments, one or more metals and/or non-metals can help preserve disorder, for example, by forming a barrier to atomic diffusion.

The presence of dissimilar atoms or molecules in the coating may also be achieved by co-depositing or sequentially depositing one or more additional metal elements (e.g., one or more additional antimicrobial metal elements). Such additional metal elements include, for example, Au, Pt, Ta, Ti, Nb, Zn, V, Hf, Mo, Si, Al, and other transition metal elements. It is believed that the presence of dissimilar metal elements (one or more primary metal elements and one or more additional metal elements) in the coating can reduce atomic diffusion and stabilize the atomically disordered structure of the coating. A coating containing dissimilar metal elements can be formed, for example, using thin film deposition equipment with multiple targets. In some embodiments, sequentially deposited layers of the metal elements are discontinuous (e.g., islands within a the primary metal). In certain embodiments, the weight ratio of the additional metal(s) to the primary metal(s) is greater than about 0.2.

While FIG. 2 shows one embodiment of a deposition system, other embodiments are possible. For example, the deposition system can be designed such that during operation the substrate moves along rollers. Additionally or alternatively, the deposition system may contain multiple energy sources, multiple targets, and/or multiple substrates. The multiple energy sources, targets and/or substrates can be, for example, positioned in a line, can be staggered, or can be in an array.

In certain embodiments, two layers of the material are deposited on the substrate to achieve an optical interference effect. Alternatively, the two layers can be formed of different materials, with the outer (top) of the two layers being formed of an antimicrobial, atomically disordered, nanocrystalline silver-containing material, and the inner of the two layers having appropriate reflective properties so that the two layers can provide an interference effect (e.g., to monitor the thickness of the outer (top) of the two layers).

The substrate can be selected as desired. The substrate may be formed of one layer or multiple layers, which may be formed of the same or different materials. In certain embodiments, the substrate can include one or more layers containing a bioabsorbable material. Bioabsorbable materials are disclosed, for example, in U.S. Pat. No. 5,423,859. In general, bioabsorbable materials can include natural bioabsorbable polymers, biosynethetic bioabsorbable polymers and synthetic bioabsorbable polymers. Examples of synthetic bioabsorbable polymers include polyesters and polylactones (e.g., polymers of polyglycolic acid, polymers of glycolide, polymers of lactic acid, polymers of lactide, polymers of dioxanone, polymers of trimethylene carbonate, polyanhydrides, polyesteramides, polyortheoesters, polyphosphazenes, and copolymers of the foregoing). Examples of natural bioabsorbable polymers include proteins (e.g., albumin, fibrin, collagen, elastin), polysaccharides (e.g., chitosan, alginates, hyaluronic acid). Examples of biosynthetic polymers include polyesters (e.g., 3-hydroxybutyrate polymers).

In some embodiments, the substrate includes multiple layers (e.g., two layers, three layers, four layers, five layers, six layers, seven layers, eight layers, nine layers, 10 layers). The layers can be laminated together (e.g., by thermal fusing, stitching and/or ultrasonic welding).

One or more layers (e.g., an outer layer) of a multi-layer substrate can be formed of a perforated (and optionally non-adherent) material (e.g., a woven material or a non-woven material) that can allow fluid to penetrate or diffuse therethrough. Such materials include, for example, cotton, gauze, polymeric nets (e.g., polyethylene nets, nylon nets, polypropylene nets, polyester nets, polyurethane nets, polybutadiene nets), polymeric meshes (e.g., polyethylene meshes, nylon meshes, polypropylene meshes, polyester meshes, polyurethane meshes, polybutadiene meshes) and foams (e.g., an open cell polyurethane foam). Examples of commercially available materials include DELNET™ P530 non-woven polyethylene veil (Applied Extrusion Technologies, Inc., Middletown, Del.), Exu-Dry CONFORMANT2™ non-woven polyethylene veil (Frass Survival Systems, Inc., NY, N.Y.), CARELLE™ material (Carolina Formed Fabrics Corp.), NYLON90™ material (Carolina Formed Fabrics Corp.), N-TERFACE™ material (Winfield Laboratories, Inc., Richardson, Tex.), HYPOL™ hydrophilic polyurethane foam (W.R. Grace & Co., NY, N.Y.).

One or more layers (e.g., an inner layer) of a multi-layer substrate can be formed of an absorbent material (e.g., a woven material or a non-woven material) formed of, for example, rayon, polyester, a rayon/polyester blend, polyester/cotton, cotton and/or cellulosic fibers. Examples include creped cellulose wadding, air felt, air laid pulp fibers and gauze. An example of a commercially available material is SONATRA™ 8411 70/30 rayon/polyester blend (Dupont Canada, Mississauga, Ontario).

One or more layers (e.g., an outer layer) of a multi-layer substrate can be formed of an occlusive or semi-occlusive material, such as an adhesive tape or polyurethane film (e.g., to secure the device to the skin and/or to retain moisture).

In some embodiments, the layers in a multi-layer substrate are laminated together (e.g., at intermittent spaced locations) by ultrasonic welds. Typically, heat (e.g., generated ultrasonically) and pressure are applied to either side of the substrate at localized spots through an ultrasonic horn so as to cause flowing of at least one of the plastic materials in the first and second layers and the subsequent bonding together of the layers on cooling. The welds can be formed as localized spots (e.g., circular spots). The spots can have a diameter of about 0.5 centimeter or less.

The shape of the substrate can generally be varied as desired. For example, the substrate can be in the shape of a film, a fiber or a powder.

The substrate/coating article can be used in a variety of articles. For example, the article can be in the shape of a medical device. Exemplary medical devices include wound closure devices (e.g., sutures, staples, adhesives), tissue repair devices (e.g., meshes, such as meshes for hernia repair), prosthetic devices (e.g., internal bone fixation devices, physical barriers for guided bone regeneration, stents, valves, electrodes), tissue engineering devices (e.g., for use with a blood vessel, skin, a bone, cartilage, a liver), controlled drug delivery systems (e.g., microcapsules, ion-exchange resins) and wound coverings and/or fillers (e.g., alginate dressings, chitosan powders). In some embodiments, the article is a transcutaneous medical device (e.g., a catheter, a pin, an implant), which can include the substrate/coating supported on, for example, a solid material (e.g., a metal, an alloy, latex, nylon, silicone, polyester and/or polyurethane). In some embodiments, the article is in the form of a patch (e.g., a patch having an adhesive layer for adhering to the skin, such as a transdermal patch).

Subsequent to deposition, the material can optionally be annealed. In general, the anneal is conducted under conditions to increase the stability (e.g., shelf life) of the material while maintaining the desired therapeutic activity of the material. In certain embodiments, the material can be annealed at a temperature of about 200° C. or less (e.g., about room temperature).

The substrate/coating is typically sterilized prior to use (e.g., without applying sufficient thermal energy to anneal out the atomic disorder). The energy used for sterilization can be, for example, gamma radiation or electron beam radiation. In some embodiments, ethylene oxide sterilization techniques are used to sterilize the substrate/coating.

Free Standing Powders

A free standing powder can be prepared by, for example, cold working or compressing to impart atomic disorder to the powder. In certain embodiments, a free standing powder is prepared by forming a coating of the material as described above, and then removing the material from the surface of the substrate. For example, the material can be scraped from the surface of the substrate by one or more scrapers. In embodiments in which the substrate moves during deposition of the material, the scrapers can remove the material as the substrate moves. The scrapers can be, for example, suspended above the substrate. Such scrapers can be, for example, weighted and/or spring loaded to apply pressure sufficient to remove the material as the substrate moves. In some embodiments (e.g., when a continuous belt is used), the scrapers can be located above the end rollers to remove the material with a reverse dragging action as the substrate rounds the end roller.

A free standing powder can be used to treat a condition in various ways. As an example, the powder can sprinkled onto the subject's skin. As another example, the powder can be inhaled using an inhaler, such as a dry powder inhaler. In some embodiments, a dry powder can be in the form of an aerosol, which contains, for example, at least about 10 (e.g., at least about 20, at least about 30) weight percent and/or at most about 99 (e.g., at most about 90, at most about 80, at most about 70, at most about 60, at most about 50) weight percent of the dry powder. In some embodiments, the aerosol can contain from about 10 to 99 (e.g., from 10 to 90, from 10 to 70, from 10 to 50) percent by weight of the dry powder.

In certain embodiments (e.g., when the free standing powder is inhaled), the average particle size of the free standing powder is selected to reduce the likelihood of adverse reaction(s) of the particles in the tissue and/or to deposit the powder onto specific anatomical locations (e.g., tissue contacted by the free standing powder during inhalation). In some embodiments, the average particle size is selected (e.g., less than about 10 microns) so that a relatively small amount of the particles get into the lower respiratory tract. In embodiments, a free standing powder can have an average particle size of less than about 10 microns (e.g., less than about eight microns, less than about five microns, less than about two microns, less than about one micron, less than about 0.5 micron) and/or at least about 0.01 micron (e.g., at least about 0.1 micron, at least about 0.5 micron).

Powder Impregnated Materials

The metal-containing material can be in the form of a powder impregnated material. Such powder impregnated materials can, for example, be in the form of a hydrocolloid having the free standing powder blended therein. A powder impregnated material can be, for example, in the form of a dressing, such as a hydrocolloid dressing.

The following examples are illustrative and not intended as limiting.

### EXAMPLES

### Example 1

### Preparation of Nanocrystalline Silver Coatings on Dressings

This example shows the preparation of a bilayer nanocrystalline silver coating on a dressing material. A high density polyethylene dressing, DELNET™ or CONFORMANT 2™ was coated with a silver base layer and a silver/oxide top layer to generate a colored anti-microbial coating having indicator value. The coating layers were formed by magnetron sputtering under the conditions set out in the following table.

| Sputtering Conditions: | Base Layer | Top Layer |
|---|---|---|
| Target | 99.99% Ag | 99.99% Ag |
| Target Size | 20.3 cm diameter | 20.3 cm diameter |
| Working Gas | 96/4 wt % Ar/O₂ | 96/4 wt % Ar/O₂ |
| Working Gas Pressure | 5.33 Pa (40 mT) | 5.33 Pa (40 mT) |
| Power | 0.3 kW | 0.15 kW |
| Substrate Temperature | 20° C. | 20° C. |
| Base Pressure | 3.0 × 10⁻⁶ Torr | 3.0 × 10⁻⁶ Torr |
| Anode/Cathode | 100 mm | 100 mm |
| Distance | | |
| Sputtering Time | 7.5-9 min | 1.5 min |
| Voltage | 369-373 V | 346 V |

The resulting coating was blue in appearance. A fingertip touch was sufficient to cause a color change to yellow. The base layer was about 900 nm thick, while the top layer was 100 nm thick.

To establish that silver species were released from the coated dressings, a zone of inhibition test was conducted. Mueller Hinton agar was dispensed into Petri dishes. The agar plates were allowed to surface dry prior to being inoculated with a lawn of Staphylococcus aureus ATCC#25923. The inoculant was prepared from Bactrol Discs (Difco, M.), which were reconstituted as per the manufacturer's directions. Immediately after inoculation, the coated materials to be tested were placed on the surface of the agar. The dishes were incubated for 24 hr. at 37° C. After this incubation period, the zone of inhibition was calculated (corrected zone of inhibition=zone of inhibition−diameter of the test material in contact with the agar). The results showed a corrected ZOI of about 10 mm, demonstrating good release of silver species.

The coating was analyzed by nitric acid digestion and atomic absorption analysis to contain 0.24+/−0.04 mg silver per mg high density polyethylene. The coating was a binary alloy of silver (>97%) and oxygen with negligible contaminants, based on secondary ion mass spectroscopy. The coating, as viewed by SEM, was highly porous and consisted of equiaxed nanocrystals organized into coarse columnar structures with an average grain size of 10 nm. Silver release studies in water demonstrated that silver was released continuously from the coating until an equilibrium concentration of about 66 mg/L was reached (determined by atomic absorption), a level that is 50 to 100 times higher than is expected from bulk silver metal (solubility≦1 mg/L).

By varying the coating conditions for the top layer to lengthen the sputtering time to 2 min, 15 sec., a yellow coating was produced. The top layer had a thickness of about 140 nm and went through a color change to purple with a fingertip touch. Similarly, a purple coating was produced by shortening the sputtering time to 1 min, to achieve a top layer thickness of about 65 nm. A fingertip touch caused a color change to yellow.

To form a three layer dressing, two layers of this coated dressing material were placed above and below an absorbent core material formed from needle punched rayon/polyester (SONTARA™ 8411). With the silver coating on both the first and third layers, the dressing may be used with either the blue coating side or the silver side in the skin facing position. For indicator value, it might be preferable to have the blue coating visible. The three layers were laminated together by ultrasonic welding to produce welds between all three layers spaced at about 2.5 cm intervals across the dressing. This allowed the dressing to be cut down to about 2.5 cm size portions for smaller dressing needs while still providing at least one weld in the dressing portion.

The coated dressings were sterilized using gamma radiation and a sterilization dose of 25 kGy. The finished dressing was packaged individually in sealed polyester peelable pouches, and has shown a shelf life greater than 1 year in this form. The coated dressings can be cut in ready to use sizes, such as 5.1×10.2 cm strips, and slits formed therein before packaging. Alternatively, the dressings may be packaged with instructions for the clinician to cut the dressing to size and form the desired length of the slit for the medical device.

Additional silver coated dressings were prepared in a full scale roll coater under conditions to provide coatings having the same properties set out above, as follows:
the dressing material included a first layer of silver coated DELNET, as set out above, laminated to STRATEX, AET, 8.0NP₂-A/QW, which is a layer of 100% rayon on a polyurethane film.
Silver Foam Dressing—three layers of silver coated high density polyethylene prepared as above, alternating with two layers of polyurethane foam, L-00562-6 Medical Foam, available from Rynel Ltd., Bootbay, Me., USA.

### Example 2

### Preparation of Nanocrystalline Silver Powders

Nanocrystalline silver powder was prepared by preparing silver coatings on silicon wafers, under the conditions set forth in the table above, and then scraping the coating off using a glass blade.

Nanocrystalline silver powder was also prepared by sputtering silver coatings on silicon wafers using Westaim Biomedical NGRC unit, and then scraping the coating off.

The sputtering conditions were as follows:

| | |
|---|---|
| Target: | 99.99% Ag |
| Target Size: | 15.24 cm × 1216.125 cm |
| Working Gas: | 75:25 wt % Ar/O₂ |
| Working Gas Pressure: | 40 mTorr |
| Total Current: | 40 A |
| Base Pressure: | 5.0 × 10⁻⁵ Torr |
| Sandvik Belt Speed: | 340 mm/min |
| Voltage: | 370 V |

The powder has a particle size ranging from 2 μm to 100 μm, with crystallite size of 8 to 10 nm, and demonstrated a positive rest potential.

### Example 3

### Preparation of Nanocrystalline Silver Coatings on Dressings

This example shows the preparation of a bilayer nanocrystalline silver coating on a dressing material. A high density polyethylene dressing, DELNET™ or CONFORMANT 2™ was coated with a silver base layer and a silver/oxide top layer to generate a colored antimicrobial coating having indicator value as described in Example 1 of the Treatment of Hyperproliferative Skin conditions examples. The coating layers were formed by magnetron sputtering under the conditions set out in the following table.

### Example 4

### Preparation of Nanocrystalline Silver Coating on HDPE Mesh

The silver coated mesh was produced, as set forth in Example 1, by sputtering silver onto Delnet, a HDPE mesh (Applied Extrusion Technologies, Inc., Middletown, Del., USA) using Westaim Biomedical TMRC unit under the following conditions:

| | |
|---|---|
| Target: | 99.99% Ag |
| Target Size: | 15.24 cm × 152.4 cm |
| Working Gas: | 99.375:0.625 wt % Ar/O₂ |
| Working Gas Pressure: | 5.33 Pascals (40 mTorr) |
| Total Current: | 22 A |
| Base Pressure: | 5.0 × 10⁻⁵ Torr |
| Sandvik Belt Speed: | 577 mm/min |
| Voltage: | 367 V |

The coating was tested and found to have a weight ratio of reaction product to silver of between 0.05 and 0.1. The dressing was non-staining to human skin.

### Example 5

### Preparation of Atomic Disordered Nanocrystalline Silver Powders

Nanocrystalline silver coatings were prepared by sputtering silver in an oxygen-containing atmosphere directly onto an endless stainless steel belt of a magnetron sputtering roll coater, or onto silicon wafers on the belt. The belt did not need to be cooled. The coatings were scraped off with the belt with suspended metal scrapers as the belt rounded the end rollers. For the coated silicon wafers, the coatings were scraped off with a knife edge. The sputtering conditions were as follows:

| | |
|---|---|
| Target: | 99.99% Ag |
| Target Size: | 15.24 cm × 1216.125 cm |
| Working Gas: | 75:25 wt % Ar/O₂ |
| Working Gas Pressure: | 5.33 Pascals (40 milliTorr) |
| Total Current: | 40 A |
| Base Pressure: | 5.0 × 10⁻⁵ Torr (range: 1 × 10⁻⁴-9 × 10⁻⁷ Torr |
| | or 1 × 10⁻²-1.2 × 10⁻⁴ Pa) |
| Sandvik Belt Speed: | 340 mm/min |
| Voltage: | 370 V |

Note -
pressure conversions to Pa herein may not be accurate, most accurate numbers are in torr, mTorr units.

The powder had a particle size ranging from 2 μm to 100 μm, with grain or crystallite size of 8 to 10 nm (i.e., nanocrystalline), and demonstrated a positive rest potential.

Similar atomic disordered nanocrystalline silver powders were formed as set forth hereinabove by magnetron sputtering onto cooled steel collectors, under conditions taught in the prior Burrell et al. patents to produce atomic disorder.

### Example 6

### Vehicle Formulations

General procedure: in a first container, polyoxyl 40 myristate, methylparaben, and water were combined and heated to 73-77° C. to form a first mixture. In a separate container, cetearyl alcohol, glycerol monostearate, stearic acid, light mineral oil, isopropyl myristate, propylparaben, and xanthan gum were combined and heated to 73-77° C. to form a second mixture. The second mixture was gradually added to the first mixture while mixing and homogenizing until a cream base is formed. Table 1 lists two sample vehicle compositions.

TABLE 1

| | |
|---|---|
| Compositions | Sample |

| % w/w | I | II |
|---|---|---|
| Water | Balance | Balance |
| Cetearyl alcohol | 3 | 5 |
| Glycerol monostearate | 4 | 4 |
| Stearic acid | 4 | 4 |
| Light mineral oil | 7 | 7 |
| Isopropyl myristate | 8 | 8 |
| Polyoxyl 40 stearate | 1 | 1 |
| Propylparaben | 0.02 | 0.02 |
| Methylparaben | 0.18 | 0.18 |
| Xanthan gum | NA | 0.5 |

### Example 7

### Cream Formulations Containing 0.5% Nanocrystalline Silver

General procedure: phase A components were combined and heated to 73-77° C. In a separate container, phase B components were combined and heated to 73-77° C. The phase B mixture was gradually added to about 80% of the phase A mixture while mixing and homogenizing until a cream base is formed. In a third container, nanocrystalline silver (phase C) was dispersed in the remaining 20% of phase A by homogenization. The nanocrystalline silver mixture was gradually added to the cream base, and mixing was continued until the cream is cooled to below 30° C. Table 2 lists the components of ten sample cream formulations, each containing 0.5% by weight nanocrystalline silver.

TABLE 2

| | Ingredient | Sample | | | | | | | | | |
|---|---|---|---|---|---|---|---|---|---|---|---|
| Phase | (% w/w) | I | II | III | IV | V | VI | VII | VIII | IX | X |
| A | Water | BL* | BL | BL | BL | BL | BL | BL | BL | BL | BL |
| B | Cetearyl alcohol | 5 | 5 | 5 | 7 | 8 | 8 | 8 | 8 | 8 | 8 |
| B | Glycerol | 4 | 6 | 6 | 6 | 6 | 6 | 8 | 8 | 6 | 6 |
| | monostearate | | | | | | | | | | |
| B | Stearic acid | 4 | 4 | 4 | 4 | 4 | 3 | 0 | 0 | 5 | 5 |
| B | Light mineral oil | 7 | 0 | 0 | 0 | 0 | 0 | 0 | 0 | 0 | 0 |
| B | White petrolatum | 0 | 7 | 7 | 7 | 10 | 8 | 8 | 8 | 8 | 8 |
| B | Isopropyl | 8 | 8 | 8 | 8 | 0 | 5 | 5 | 5 | 5 | 5 |
| | myristate | | | | | | | | | | |
| A | Polyoxyl 40 | 1 | 1.2 | 1.5 | 1.5 | 1.5 | 1.5 | 1.5 | 1.5 | 1.5 | 1.5 |
| | stearate | | | | | | | | | | |
| B | Propylparaben | 0.02 | 0.02 | 0.02 | 0.02 | 0.02 | 0.02 | 0.02 | 0.02 | 0.02 | 0.02 |
| A | Methylparaben | 0.18 | 0.18 | 0.18 | 0.18 | 0.18 | 0.18 | 0.18 | 0.18 | 0.18 | 0.18 |
| B | Xanthan gum | 0.2 | 0.2 | 0.2 | 0.1 | 0.1 | 0.1 | 0.1 | 0.1 | 0.1 | 0.1 |
| A | PEG 400 | 0 | 0 | 3 | 5 | 5 | 6 | 6 | 6 | 6 | 6 |
| C | Nanocrystalline | 0.5 | 0.5 | 0.5 | 0.5 | 0.5 | 0.5 | 0.5 | 0.5 | 0.5 | 0.5 |
| | silver | | | | | | | | | | |

*BL: balance

Sample I had a smooth texture, and was dark-colored. Sample II had a smooth texture, and was lighter in color than sample I. Sample III had a smooth texture, and was lighter in color than sample II. Sample IV had a smooth texture. Sample V had silver particulates. Samples VII and VIII was smooth textured and light grey in color. Sample IX and X were light grey in color and smooth.

### Example 8

### Cream Formulations Including Nanocrystalline Silver

Cream formulations including 0.25, 1.00, or 2.00 percent by weight nanocrystalline silver were prepared according to the general procedure of Example 7. Table 3 lists the components of the cream formulations.

TABLE 3

| | | Sample | | |
|---|---|---|---|---|
| Phase | Compositions % w/w | I | II | III |
| B | Cetearyl alcohol | 8 | 8 | 8 |
| B | Glycerol monostearate | 6 | 6 | 6 |
| B | Stearic acid | 5 | 5 | 5 |
| B | White petrolatum | 8 | 8 | 8 |
| B | Isopropyl myristate | 5 | 5 | 5 |
| A | Polyoxyl 40 stearate | 1.5 | 1.5 | 1.5 |
| B | Propylparaben | 0.02 | 0.02 | 0.02 |
| A | Methylparaben | 0.18 | 0.18 | 0.18 |
| B | Xanthan gum | 0.1 | 0.1 | 0.1 |
| A | PEG 400 | 6 | 6 | 6 |
| C | Nanocrystalline silver | 0.25 | 1 | 2 |

### Example 9

### Creams Containing Titanium Dioxide, Stearic Acid-Coated Titanium Oxide, or Zinc Oxide

Either 0.15 gram of zinc oxide or stearic acid-coated titanium dioxide were mixed with 5.0 grams of 0.5% nanocrystalline cream of Example 7, Sample III. The stearic acid-coated titanium dioxide containing creams looked lighter in color. A comparison of uncoated titanium dioxide and stearic acid-coated titanium dioxide was also carried out by mixing either 2.5 grams of stearic acid-coated titanium dioxide or 2.5 grams of uncoated titanium dioxide directly with 50 grams of 0.5% nanocrystalline silver cream. The stearic acid-coated titanium dioxide containing cream was lighter in color and smoother than the cream including the uncoated titanium dioxide cream.

Creams including titanium oxide were prepared according to the following procedure: Phase A ingredients were combined and heated to 73-77° C. in a first container, phase B ingredients were combined and heated to 73-77° C. in a second container. About 80% of the phase A mixture was transferred to the phase B container with constant mixing and homogenizing to form a cream base. Nanocrystalline silver was dispersed in the remaining phase A mixture by homogenization for 5-10 minutes.

The nanocrystalline silver mixture was then dispersed into the cream base. Water was mixed into the cream base, and PEG 400 was added to the cream, which was mixed until the cream cooled to below 30° C. All the creams were smooth, homogeneous, and had good viscosity. Table 4 lists the composition of the cream formulations.

TABLE 4

| | | Sample | | | | | |
|---|---|---|---|---|---|---|---|
| Phase | Composition | I | II | III | IV | V | VI |
| A | Purified water | balance | balance | balance | balance | balance | balance |
| A | Polyoxyl 40 stearate | 1.5 | 1.5 | 1.5 | 1.5 | 1.5 | 1.5 |
| B | Cetearyl alcohol | 5 | 5 | 5 | 5 | 5 | 5 |
| B | Glycerol | 4 | 4 | 4 | 4 | 4 | 4 |
| | monostearate | | | | | | |
| B | Stearic acid | 5 | 5 | 5 | 5 | 5 | 5 |
| B | White petrolatum | 7 | 6 | 5 | 5 | 5 | 5 |
| B | Isopropyl myristate | 5 | 7 | 6 | 6 | 6 | 6 |
| B | Propylparaben | 0.02 | 0.02 | 0.02 | 0.02 | 0.02 | 0.02 |
| A | Methylparaben | 0.18 | 0.18 | 0.18 | 0.18 | 0.18 | 0.18 |
| B | Xanthan Gum | 0.1 | 0.1 | 0.05 | 0.05 | 0.05 | 0.05 |
| B | Titanium dioxide | 5 | 5 | 5 | 5 | 5 | 5 |
| C | Nanocrystalline | 1 | 1 | 0.5 | 0.25 | 2 | 0 |
| | silver | | | | | | |
| D | PEG 400 | 6 | 6 | 6 | 6 | 6 | 6 |

### Example 10

### Cream Formulations Including Nanocrystalline Silver

A cream including 0%, 0.5%, 1.0%, or 2% nanocrystalline silver was prepared according to the following procedure: phase B emollients and co-surfactants were melted and combined in a first container. Titanium dioxide and benzyl alcohol were dispersed into the melted mixture in the first container. Polyoxyl 40 stearate was then dispersed in water in a second container. Most of the polyoxyl 40 stearate mixture was then transferred into the first container with constant mixing to form a cream base. Nanocrystalline silver and/or iron oxide was then dispersed and homogenized in the remaining polyoxyl 40 stearate mixture. The nanocrystalline silver mixture was then transferred to the cream base with constant mixing until a uniform cream is formed. Table 5A and 5B list the compositions of the cream formulations.

TABLE 5A

| | | Sample | | | |
|---|---|---|---|---|---|
| Phase | Ingredient % w/w | I | II | III | IV |
| A | Purified water | balance | balance | balance | balance |
| A | Polyoxyl 40 stearate | 1.5 | 1.5 | 1.5 | 1.5 |
| B | Cetyl alcohol | 4 | 4 | 4 | 4 |
| B | Glycerol monostearate | 3 | 3.5 | 3 | 3.5 |
| B | Stearic acid | 5.5 | 5.5 | 5 | 5.5 |
| B | White petrolatum | 3 | 3 | 3 | 3 |
| B | Isopropyl myristate | 4 | 4 | 4 | 4 |
| B | Benzyl alcohol | 0.5 | 0.5 | 0.5 | 0.5 |
| B | Titanium dioxide | 5 | 5 | 5 | 5 |
| C | Nanocrystalline silver | 2 | 0.5 | 0 | 1 |
| D | PEG 400 | 6 | 6 | 6 | 6 |
| | pH value of samples | 6.21 | 5.88 | 5.17 | 6.22 |

TABLE 5B

| | | | 0.5% | 1.0% | 2.0% |
|---|---|---|---|---|---|
| | | Placebo | NCS | NCS | NCS |
| | Ingredient | % w/w | % w/w | % w/w | % w/w |
| A | Purified water USP | 65.99 | 67.35 | 68.1 | 68.25 |
| A | Polyoxyl 40 stearate | 1.5 | 1.5 | 1.5 | 1.5 |
| B | White petrolatum | 3.5 | 2.5 | 2 | 1.5 |
| B | Isopropyl myristate | 4 | 4 | 4 | 4 |
| B | Cetyl alcohol | 4 | 4 | 4 | 4 |
| B | Glyceryl | 4 | 3.2 | 2.5 | 2 |
| | monostearate | | | | |
| B | Stearic acid | 5 | 5 | 5 | 5 |
| B | Titanium dioxide | 5 | 5 | 5 | 5 |
| B | Benzyl alcohol | 0.75 | 0.75 | 0.75 | 0.75 |
| C | Nanocrystalline | 0 | 0.5 | 1 | 2 |
| | silver | | | | |
| C | Iron oxide | 0.26 | 0.2 | 0.15 | 0 |
| D | PEG 400 | 6 | 6 | 6 | 6 |

### Example 11

### Viscosity Tests of the Cream Compositions

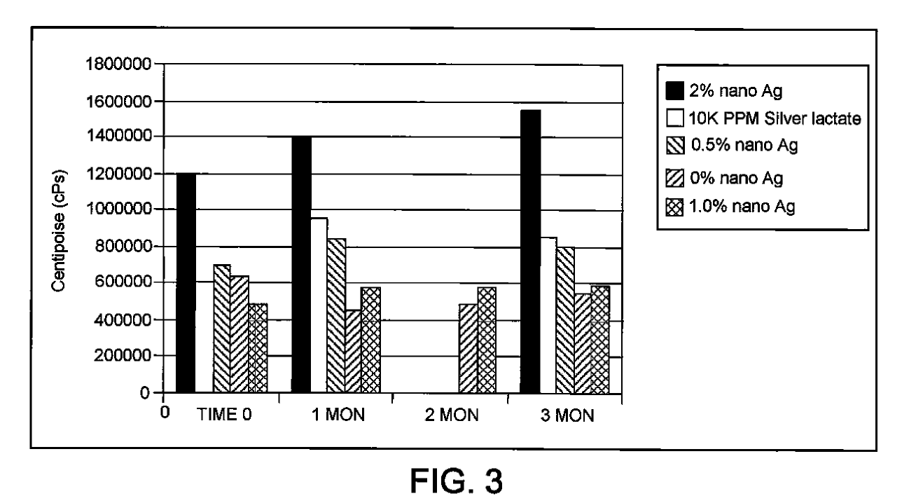

The viscosities of the creams were measured using a Brookfield RV II pro viscometer with T-D spindle measured at 1.0 rpm. FIG. 3 shows the viscosity variation of the creams from Table 5A, stored for three months at 40° C. and 75% relative humidity. The 2% cream from Table 5A was stable maintained a relatively constant viscosity over three months.

### Example 12

### Water Solubility Tests

Five grams of 0%, 1%, or 2% nanocrystalline silver creams of Table 5B were thoroughly mixed with 20 grams or 45 grams of de-ionized water. The mixtures were centrifuged for 45 minutes at 3600 rpm to separate the aqueous phase from the oil and solid phases. The amounts of silver recovered from the aqueous phase were analyzed by atomic absorption spectroscopy. The 1% cream and the 2% cream had similar soluble silver levels, and had an average level of soluble silver in the range from 0.02 to 0.07%. Table 6 shows the water solubility levels of silver for the creams, obtained from two tests.

TABLE 6

| | Water Soluble | Water Soluble |
|---|---|---|
| Nanocrystalline | Silver (%) | Silver (%) |
| silver (% w/w) | Test 1 | Test 2 |
| 2 | 0.02 | 0.005* |
| 2 | 0.03 | 0.02 |
| 2 | 0.03 | 0.02 |
| 2 | 0.03 | 0.02 |
| 2 | 0.07 | 0.07 |
| 2 | 0.07 | 0.13* |
| 1 | 0.03 | 0.05 |
| 1 | 0.03 | 0.03 |
| 0 | <LOQ** | <LOQ |
| 2 | NA | 0.02 |
| 2 | NA | 0.04 |

*Out-lier;
**limit of quantitation.

### Example 13

Compatibility of Nanocrystalline Silver with Excipients

A 10% aqueous solution of each excipients was mixed with nanocrystalline silver in a glass vial and kept at 40° C. overnight. Table 7 shows the reaction of nanocrystalline silver with various excipients.

TABLE 7

| | Reaction |
|---|---|
| Excipient | with nanocrystalline silver |
| Propylene glycol | Discoloration |
| Transcutol | Grey color - no discoloration |
| PEG 400 | Grey color - no discoloration |
| HPMC K100M | Some black precipitate |
| Xantural 75 | Light orange + hazy grey |
| Methylparaben | Brown discoloration |
| Propylparaben | Green discoloration |
| Sodium benzoate | White precipitate |
| Hydroxypropylcellulose (HPC) | Dark grey/red discoloration |
| Methyl cellulose A4M | Discoloration |
| Sodium carboxymethylcellulose | Discoloration |
| Sodium parabens | Precipitation |
| Carbopol 934 | Precipitation |
| Carageena NF | Discoloration |
| Hexylene glycol | Grey - no discoloration |
| Hexylene glycol, EDTA, and parabens | White precipitate |
| Hexylene glycol and parabens | Grey color - no discoloration |
| H₂O₂ | Brown discoloration after drying |
| Ag and EDTA | Purple/brown after drying |
| Methyl parabens and propyl parabens, | pink |
| polyoxyl 40 stearate, and HPC | |
| Benzyl alcohol | Grey/slightly red |
| Polyoxyl 40 stearate | Grey color - no discoloration |
| Cetearyl alcohol | Light brown/green/black |
| Stearic acid | Grey color - no discoloration |
| Isopropyl myristate | Grey color - no discoloration |
| Glycerol monostearate | Brown discoloration |
| Cetyl alcohol | Grey color - no discoloration |
| Xantham gum | Brown discoloration |
| Stearic acid coated titanium dioxide | Light grey/dark grey color |
| White petrolatum | Grey color - no discoloration |

### Example 14

### Fabric Staining Tests

To test the theory that a cream containing a metal-containing material can be removed from most fabrics through normal washing, a worst case test was arranged. Seven kinds of white fabrics were tested with the 2% cream to assess the ease of removal and staining properties of the cream. The fabrics were 100% cotton terry cloth, 65:35 broad cloth polyester-cotton blend, 100% cotton waffle weave, 100% combed cotton, 50:50 knit polyester-cotton blend, 100% polyester, and a wool-blend. The fabrics were stained with the 2% nanocrystalline silver-containing cream of Table 5B by applying 179 mg of the 2% active cream (containing about 3.6 mg of nanocrystalline silver) to a 28 cm² area of each type of fabric. The fabrics were then allowed to stand for two days to allow the creams to adequately dry. The fabrics were laundered in either cold, warm, or hot water using the gentle, normal or heavy cycles using a number of commonly available detergents: Tide™, Tide with Bleach Alternative™, Dreft™ (ultra detergent for babies 0-18 months), Costco™ brand detergent, Woolite™, and a combination of Shout Action Gel™ stain remover and Tide™. The amount of detergent used was per the manufacturer recommendations for “heavily soiled” clothing.

| Material/Equipment | Provider |
|---|---|
| 2% nanocrystalline silver cream | |
| Petri Dish 60 × 15 mm | VWR (Cat #: 25384-060) |
| 7 Commercial fabrics | Fabrics were purchased at Fabric Place in |
| 100% Wool | Woburn, MA. |
| 50/50 Polyester/Cotton | |
| 65/35 Polyester/Cotton | |
| 100% Cotton (waffle weave) | |
| 100% Cotton (terry cloth) | |
| 100% Combed Cotton (T-shirt) | |
| 100% polyester | |
| ColorQuest XE | Hunter Lab: S/N COX2677 |
| Universal Software v 4.10 | |
| Manual v 2.5 | |
| 50 mL Pyrex beakers or 50 mL Erlenmeyer flasks | |
| Watch glasses | |
| 50 mL centrifuge tubes with screw caps | VWR |
| Hot plates | VWR S/N: 050720007 |
| Hot plates | Fisher Scientific S/N: 502N0071 |
| Flame Atomic Absorption Spectrometry AA200 | Perkin Elmer S/N: 200S3062004 |
| Concentrated nitric acid (trace metal grade) | EMD, Lot Numbers: 44123 and 45105 |
| Concentrated sulfuric acid (trace metal grade) | EMD, Lot Number: 45105 |
| High-purity water (18.2 MΩ.cm) | Barnstead Diamond Unit # 2 |
| Mesh Lingerie bag (100% polyester, 45.7 cm × 38 cm) | Target Minneapolis, MN |
| Tide/Clean Breeze | Procter & Gamble Cincinnati, OH 45202 (Lot. |
| | 95393503) |
| Tide with Bleach Alternative/Clean Breeze | Procter & Gamble Cincinnati, OH 45202 (Lot. |
| | 95398409) |
| Dreft (for babies 0-18 months) | Procter & Gamble Cincinnati, OH 45202 |
| | (Lot.95091551) |
| Ultra/New Fresh and Clean | Costco Brand, Kirkland Signature, Seattle WA |
| | Item # 38722) |
| Woolite Original Fabric Wash | Reckitt Benckiser, Inc. Parsippany, NJ 07054 |
| Shout Action Gel | S.C Johnson & Son, Inc. WI 53403 |

Color Quest XE Parameters:

| | |
|---|---|
| Small area view | 0.375″ |
| Port size | RSIN Mode |
| Nominal | UV Filter |

Fabric Measured before Cream Applied: each fabric was cut into a 3×4″ square. Each square was sandwiched between the lid and base of the Petri dish. The lightness value for each square of fabric was measured by placing the Petri dish against the Color Quest's specular, and secured in place with the sample clamp. A color measurement was taken for each sample, and two additional measurements of each sample were also taken by rotating the dish 45°, to expose a slightly different surface. The Color Quest was calibrated before the procedure, and the green tile analyzed to calibrate the color standards after every 25 sample analyses. Standardization was performed in accordance with the manufacturer's recommendations.

Using a plastic ruler, nanocrystalline silver cream was squeezed from its tube, and a 1 cm² (˜179 mg) was measured and sliced from the tube. The 1 cm² cream sample was smeared on the top of the Petri dish by making a circular motion with an index finger. The dish was then pressed against the fabric. To ensure that full amount of the cream was being uniformly applied to each piece of fabric, the dish was rotated in a clockwise motion. After the cream was applied to each fabric sample, it was allowed to dry for 5 min.

After the stain had dried, each fabric was placed sandwiched between the lid and base of the Petri dish, and place against the Color Quest's specular, and secured in place with the sample clamp. Color measurements were taken for each sample; 2 more measurements of each sample were also taken by rotating the dish 45°, to expose a slightly different sample surface.

Samples were put into a mesh lingerie bag; and taken to a commercial laundromat for cleaning using the 4 detergents:

Tide

Tide with Bleach Alternative

Dreft

Costco's (Generic) Brand

Fabrics were washed with 3 water temperatures (hot, cold, and warm), and 3 wash cycles (Gentle, Heavy, and Normal). In each case, detergent was used according to the manufacturer's recommendations.

Stain removal from various fabrics using the various different approaches was assessed using a variety of techniques:

A. Visual Observation Recorded Using Digital Photographs

A set of the seven fabrics were photographed prior to the application of cream. A photograph of a set of the seven fabrics was recorded after a typical application of cream. Finally, photographs were taken of sets of the fabrics after they were washed using the various conditions. A visual examination could then be performed to determine if the fabrics were clean, using the fabrics with no cream, to aid in the evaluation.

B. Color Analyses Using Quantitative Color Measurements

Color analysis was performed to obtain the baseline values for the fabrics, prior to the application of any cream. Color analysis was performed after the application of the cream. Finally, color analysis was performed after the fabrics were washed. In all cases, quantitative color values were obtained.

Note: The Lightness Value for the washed fabric was compared to the value of the corresponding value of the fabric prior to the application of cream.

C. Assay for Silver Using Atomic Spectroscopy

The levels of silver remaining in the fabric were determined by analyzing the fabrics after they were laundered. This approach has the ability to support data generated by both visual observations and quantitative color, by specifically looking for the presence/absence of silver.

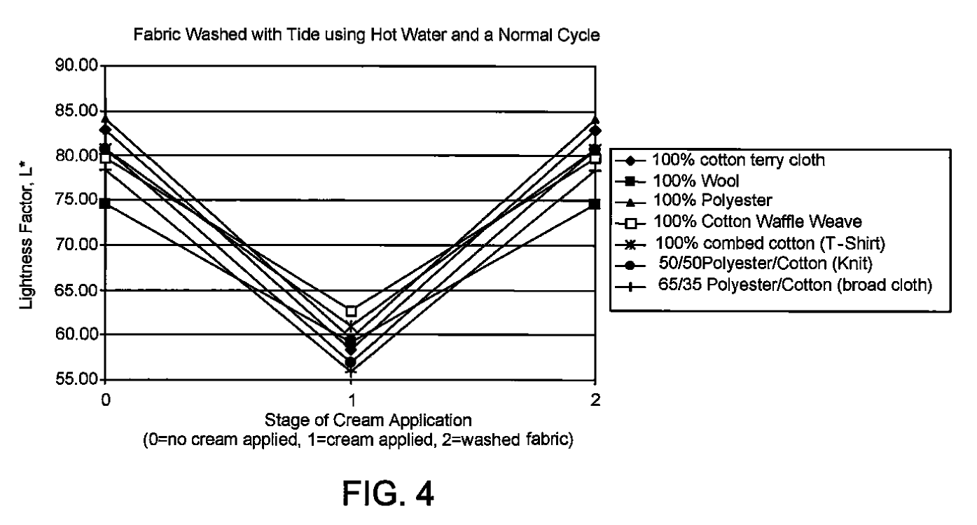

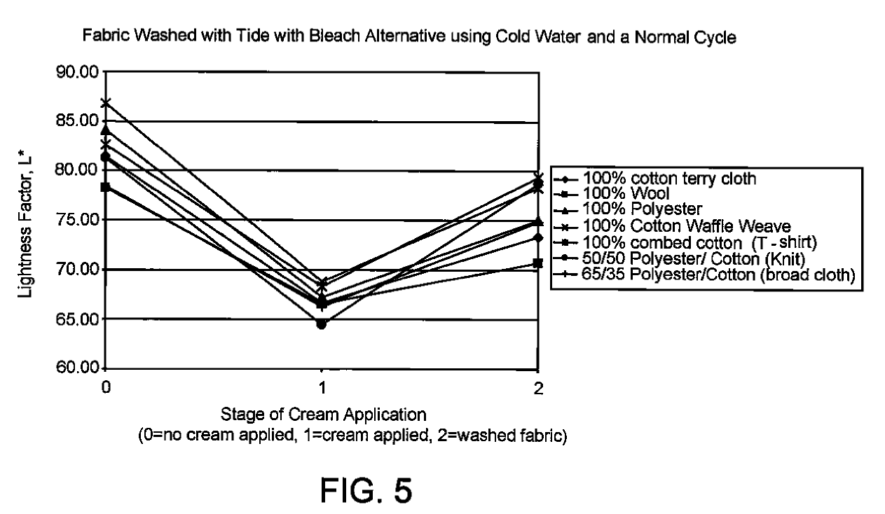

FIGS. 4 and 5 show representative quantitative color measurements of the fabric samples prior to staining, at staining, and after washing. Referring to FIG. 4, fabrics washed with Tide™ using hot water and a normal cycle had approximately the same lightness factor after washing. Referring to FIG. 5, fabrics washed with Tide with Bleach Alternative™ using cold water and a normal cycle were not fully clear of the silver-containing cream. The amount of silver on the fabrics were also measured using atomic spectroscopy, following a nitric acid digest of the fabric samples both before and after washing. The average amount of silver on the fabric were from 1.6-3.3 mg, while the amount of silver after washing ranged from 0.005 mg for a very well-cleaned fabric to 0.94 mg for a partially cleaned fabric. Table 8 shows the fabric cleaning test results for various detergents, at various temperatures and wash cycles. A well-cleaned fabric is designated by +, while a stained fabric is designated by —.

TABLE 8

| | Cold water | | | Warm water | | | Hot water | | |
|---|---|---|---|---|---|---|---|---|---|
| | Gentle | Normal | Heavy | Gentle | Normal | Heavy | Gentle | Normal | Heavy |
| Tide ™ | | | | | | | | | |
| 100% cotton | − | + | − | − | + | + | + | + | + |
| terry cloth | | | | | | | | | |
| 65:35 | − | − | − | − | − | + | + | + | + |
| polyester/cotton | | | | | | | | | |
| 100% cotton | − | − | − | − | − | − | + | + | + |
| waffle weave | | | | | | | | | |
| 100% combed | − | − | − | − | − | − | + | + | + |
| cotton | | | | | | | | | |
| 50:50 | − | − | − | − | − | − | + | + | + |
| polyester/cotton | | | | | | | | | |
| 100% polyester | + | + | + | − | + | + | + | + | + |
| 100% wool | − | − | − | − | + | − | + | + | + |
| Tide with | | | | | | | | | |
| bleach | | | | | | | | | |
| alternative ™ | | | | | | | | | |
| 100% cotton | + | + | + | + | + | + | + | + | + |
| terry cloth | | | | | | | | | |
| 65:35 | − | − | − | + | + | + | + | + | + |
| polyester/cotton | | | | | | | | | |
| 100% cotton | − | − | − | − | − | − | + | + | + |
| waffle weave | | | | | | | | | |
| 100% combed | − | − | − | − | − | − | + | + | + |
| cotton | | | | | | | | | |
| 50:50 | − | − | − | + | + | + | + | + | + |
| polyester/cotton | | | | | | | | | |
| 100% polyester | + | + | + | + | + | + | + | + | + |
| 100% wool | − | − | + | + | + | + | + | + | + |
| Dreft ™ | | | | | | | | | |
| 100% cotton | − | + | + | + | + | + | + | + | + |
| terry cloth | | | | | | | | | |
| 65:35 | − | − | − | + | + | − | + | + | + |
| polyester/cotton | | | | | | | | | |
| 100% cotton | − | − | − | − | − | − | + | + | + |
| waffle weave | | | | | | | | | |
| 100% combed | − | − | − | − | − | − | + | + | + |
| cotton | | | | | | | | | |
| 50:50 | − | − | − | − | − | + | + | + | + |
| polyester/cotton | | | | | | | | | |
| 100% polyester | − | + | + | + | + | + | + | + | + |
| 100% wool | − | − | − | − | − | + | + | + | + |
| Costco brand ™ | | | | | | | | | |
| 100% cotton | − | − | + | − | + | − | + | + | + |
| terry cloth | | | | | | | | | |
| 65:35 | − | − | − | − | − | + | + | + | + |
| polyester/cotton | | | | | | | | | |
| 100% cotton | − | − | − | − | − | − | + | + | + |
| waffle weave | | | | | | | | | |
| 100% combed | − | − | − | − | − | − | − | + | + |
| cotton | | | | | | | | | |
| 50:50 | − | − | − | − | + | − | − | + | + |
| polyester/cotton | | | | | | | | | |
| 100% polyester | − | − | + | − | + | + | + | + | + |
| 100% wool | − | − | − | − | − | − | + | + | + |
| Woolite ™ | | | | | | | | | |
| 100% cotton | − | + | N/A | N/A | N/A | N/A | N/A | N/A | N/A |
| terry cloth | | | | | | | | | |
| 65:35 | − | − | N/A | N/A | N/A | N/A | N/A | N/A | N/A |
| polyester/cotton | | | | | | | | | |
| 100% cotton | − | − | N/A | N/A | N/A | N/A | N/A | N/A | N/A |
| waffle weave | | | | | | | | | |
| 100% combed | − | − | N/A | N/A | N/A | N/A | N/A | N/A | N/A |
| cotton | | | | | | | | | |
| 50:50 | − | − | N/A | N/A | N/A | N/A | N/A | N/A | N/A |
| polyester/cotton | | | | | | | | | |
| 100% polyester | + | + | N/A | N/A | N/A | N/A | N/A | N/A | N/A |
| 100% wool | − | − | N/A | N/A | N/A | N/A | N/A | N/A | N/A |
| Tide ™ and | | | | | | | | | |
| Shout ™ | | | | | | | | | |
| 100% cotton | + | + | + | + | + | + | N/A | N/A | N/A |
| terry cloth | | | | | | | | | |
| 65:35 | + | + | + | + | + | + | N/A | N/A | N/A |
| polyester/cotton | | | | | | | | | |
| 100% cotton | + | + | + | + | + | + | N/A | N/A | N/A |
| waffle weave | | | | | | | | | |
| 100% combed | + | + | + | + | + | + | N/A | N/A | N/A |
| cotton | | | | | | | | | |
| 50:50 | + | + | + | + | + | + | N/A | N/A | N/A |
| polyester/cotton | | | | | | | | | |
| 100% polyester | + | + | + | + | + | + | N/A | N/A | N/A |
| 100% wool | + | + | + | + | + | + | N/A | N/A | N/A |

### Example 15

### Nanodispersion Including Nanocrystalline Silver Material

A nanodispersion having nanocrystalline silver was prepared by sonicating 500 mg of nanocrystalline silver with 500 mg of PVA and 99 ml of water, and sonicated for 10 to 30 minutes using a Hielscher UP400S or a Sonifier Model #250 probe ultrasonicator. The nanodispersion had suspended particles having a maximum dimension of 255 nm. The size of the particles were measured by light scattering. The particle size distribution ranged from 32.7 nm to 255 nm, with a maximum number of particles having a diameter of about 50 nm.

### Example 16

### In Vitro MIC Assays for Nanocrystalline Silver

The efficacy of nanocrystalline silver was determined by Minimal Inhibitory Concentration (MIC) assays. The spectrum of activity of nanocrystalline silver encompasses Gram-positive, Gram-negative, and fungal pathogens, and nanocrystalline silver is as effective against antibiotic-, antiseptic-, and multidrug-resistant bacteria as it is against drug-sensitive bacteria.

Bacteria were grown overnight on nutrient-containing agar. Bacteria from the plate culture were resuspended in nutrient broth to a density equivalent to a McFarland 0.5 standard, as assessed using a Spectronic 20D+ spectrophotometer (Thermo Electron Corporation, Waltham, Mass.). This suspension was diluted 1:5 in nutrient broth, and the 1:5 dilution was incubated at 37° C. until the bacterial density equaled the McFarland 0.5 (or the McFarland 1.0) standard, corresponding to a log-phase bacterial culture. (Note: some bacterial species which are slow-growing were used as 2-3 day growth instead of log-phase cultures). Nanocrystalline silver was added to 0.1 M lactate buffer, pH 4.0 or water, 6% lecithin, and was sonicated and passed through a 0.2-μm cellulose acetate filter (Corning, N.Y.). The concentration of silver in the final solution/nanodispersion was verified by atomic absorbance spectroscopy. Stock solutions/nanodispersions of nanocrystalline silver were added to wells in a 96-well microtiter plate, and were serially diluted by transferring 100 μl of each well into another well containing 100 μl fresh nutrient broth. Bacterial cultures were then diluted 1:30, McFarland 1.0 cultures were diluted 1:60 and added to the serial dilutions of silver formulations. The cultures were incubated for 18 hours at 37° C. and bacterial growth assessed by optical density at at 625 nm, on a Multiskan Ascent spectrophotometer (Thermo Electron Corporation, Waltham, Mass.). Alternatively, growth was assessed by visual observation when automated readout was not possible.

Each serial dilution was then inoculated with 10 μl of the 1:30 (or 1:60) diluted bacterial suspension. Plates were incubated 16-24 hours at 37° C. and growth was assessed by measuring the optical density of the cultures at 620 nm. Each assay was performed in quadruplicate, and the MIC was taken as the concentration of antimicrobial at which none of the four quadruplicate tests demonstrated growth.

For anaerobic bacteria (Propionibacterium, Clostridium, Porphyromonas, and Bacteroides species), the following modification to the MIC assay was used: bacteria cultured in cooked meat medium supplemented with hemin and vitamin K (Remel) were diluted in Brucella Broth (BD) to a density matching a McFarland 0.5 standard. This suspension was then further diluted 1:30 in Brucella Broth. This 1:30 dilution was used as the inoculum for the MIC assay. The serial dilutions of nanocrystalline silver in the microtiter plate used Brucella Broth (BD) supplemented with 5 μg ml-1 hemin (Sigma), 2.5 ng ml-1 vitamin K (Sigma), and 5% laked horse blood (Cedar Lane Laboratories) as the diluent. All cultures were done at 37° C. in an atmosphere of 80% N₂, 10% H₂, 10% CO₂, and growth was assessed visually. Each assay was performed in quadruplicate, and the MIC was taken as the concentration of antimicrobial at which none of the four quadruplicate tests demonstrated growth.

For Helicobacter pylori the macrodilution MIC assay was used: H. pylori was cultured on blood agar slants (PML Microbiological Laboratories, Inc., Wilsonville, Oreg.) at 37° C. under microaerophilic conditions, and were harvested by vortexing, adjusted to an optical density equivalent to a McFarland 0.5 standard, and used as the inoculum for the assay. Stock solutions of nanocrystalline silver were diluted in tryptic soy broth (TSB), were added to test tubes, and were serially diluted by transferring 1 ml of each tube into another tube containing 1 ml fresh TSB. Each serial dilution (0.5 ml) was then added in duplicate to blood agar slants, and the slants were then inoculated with 50 μl of the bacterial suspension. Slants were incubated 48-72 hours at 37° C. under microaerophilic conditions, and growth was assessed visually. Each assay was performed in duplicate, and the MIC was taken as the concentration of antimicrobial at which neither duplicate tests demonstrated growth.

For fungal species (Aspergillis, Candida, Cryptococcus, and Trichophyton species) the following modification to the MIC assay was used: fungi cultured on Potato Dextrose Agar or Sabouraud Dextrose Agar were harvested with a sterile disposable inoculating loop, and were resuspended in Sabouraud Dextrose Broth (SDB; BD) or Potato Dextrose Broth (PDB; BD) to an optical density equivalent to a McFarland 0.5 standard. This suspension was then diluted 1:1000 in fresh SDB or PDB, and the dilution used as the inoculum for the assay.

Stock solutions of nanocrystalline silver or antiseptics were added to wells in a 96-well microtiter plate, and were serially diluted by transferring 100 μl of each well into another well containing 100 μl fresh SDB or PDB. Each serial dilution was then inoculated with 100 μl of the 1:1000 diluted fungal suspension. Plates were incubated 48-72 hours at 23° C. and growth was assessed by measuring the optical density of the cultures at 620 nm, or visually in cases where automated readout was not possible. Each assay was performed in quadruplicate, and the MIC was taken as the concentration of antimicrobial at which none of the four quadruplicate tests demonstrated growth.

Table 9 shows representative MIC values for nanocrystalline silver against gram-positive bacteria, gram-negative bacteria, and fungi. All micro-organisms were tested by a microdilution MIC assay, except for H. pylori, which was tested by macrodilution MIC. Nanocrystalline silver possessed broad spectrum anti-microbial properties.

TABLE 9

| | | MIC (μg/ml) |
|---|---|---|
| Gram positive bacteria | Staphylococcus aureus | 9 |
| | Staphylococcus epidermidis | 1 |
| | Staphylococcus hominis | 4 |
| | Staphylococcus saprophyticus | 8 |
| | Staphylococcus warneri | 4 |
| | Staphylococcus haemolyticus | 8 |
| | Streptococcus pyogenes | 1 |
| | Streptococcus pneumoniae | 1 |
| | Streptococcus agalactiae | 2 |
| | Streptococcus mitis | 4 |
| | Streptococcus bovis | 2 |
| | Propionibacterium acnes | 250 |
| | Clostridium difficile | 9-17 |
| Gram negative bacteria | Pseudomonas aeruginosa | 1-2 |
| | Pseudomonas stutzeri | 4 |
| | Burkholderia multivorans | 2 |
| | Burkholderia dolosa | 2-4 |
| | Citrobacter freundii | 4 |
| | Enterobacter cloacae | 4 |
| | Klebsiella pneumoniae | 1-4 |
| | Proteus mirabilis | 4 |
| | Proteus vulgaris | 1 |
| | Serratia marcescens | 8 |
| | Escherichia coli | 1-64 |
| | Shigella flexneri | 2 |
| | Helicobacter pylori | 1* |
| | Morganella morganii | 8 |
| | Porphyromonas gingivalis | 31 |
| | Bacteroides fragilis | 31 |
| | Bacteroides thetaiotaomicron | 120 |
| Fungi | Aspergillus fumigatus | 16-32 |
| | Aspergillis niger | 16 |
| | Candida albicans | 9-16 |
| | Cryptococcus neoformans | 31 |
| | Trichophyton rubrum | 31 |
| | Trichophyton mentagrophytes | 31 |

*Helicobacter pylori was tested in a macrodilution MIC assay

Because antibiotic resistance has been correlated with heavy metal resistance, a collection of antibiotic-resistant organisms were surveyed for their susceptibility to nanocrystalline silver. None of the antibiotic- or antiseptic-resistant isolates that were examined demonstrated cross-resistance to nanocrystalline silver.

As shown in Tables 10, 11, 12, and 13, average MIC values (μg/ml±s.d.) were obtained for nanocrystalline silver against various antibiotic or antiseptic-resistant and susceptible Staph. aureus isolates. Table 10 shows MIC values for nanocrystalline silver against methicillin-resistant and -susceptible Staph. aureus. Nine samples of methicillin-resistant Staph. aureus and 11 samples of methicillin susceptible Staph. aureus were tested. (P=0.70 by t-test)

TABLE 10

| Methicillin sensitivity | nanocrystalline silver |
|---|---|
| for S. aureus | MIC |
| Sensitive | 10 ± 4 |
| Resistant | 10 ± 4 |

Table 11 shows MIC values (μg/ml) for nanocrystalline silver against vancomycin resistant enterococci (VRE)/and vancomycin-resistant Staphylococcus aureus (VRSA) bacterial strains. Vancomycin-sensistive Staph aureus (VSSA) was used for comparison.

TABLE 11

| | | MIC Nanocrystalline silver |
|---|---|---|
| Species | Phenotype | (μg/ml) |
| Enterococcus faecalis | VRE | 10 |
| Staphylococcus aureus | VRSA | 10 |
| Staphylococcus aureus | VSSA | 10 |

Benzalkonium chloride (BAC) is an antiseptic that is commonly used in healthcare environments to control the spread of infection. Table 12 shows MIC values (μg/ml) obtained for nanocrystalline silver against BAC-resistant P. aeruginosa isolate NP221 and BAC-sensitive isolates NP220 and 222.

TABLE 12

| | BAC MIC | nanocrystalline silver MIC |
|---|---|---|
| P. aeruginosa Isolate | (μg/ml) | (μg/ml) |
| NP220 | 63 | 3 |
| NP221 | 4000 | 6 |
| NP222 | 63 | 6 |

The inducible macrolide-, lincoasamide-, streptogramin-resistant (iMLS) phenotype is mettlesome in infection management since bacteria with this characteristic are easily mistaken for antibiotic-susceptible. Table 13 shows the average MIC values (μg/ml±s.d.) obtained for nanocrystalline silver against Staph. aureus isolates with or without the iMLS resistance phenotype. The data show that iMLS bacteria are just as susceptible to nanocrystalline silver as are antibiotic-sensitive bacteria. P=0.41 by t-test

TABLE 13

| | | nanocrystalline silver |
|---|---|---|
| Inducible resistance | N | MIC |
| Positive | 4 | 11 ± 6 |
| Negative | 16 | 9 ± 3 |

### Example 17

### ATP Modulation Study

The amount of stored biological energy in the form of the molecule adenosine triphosphate (ATP) is frequently taken as an indication of the health of bacterial cells; ATP level can therefore be used as a surrogate measure of antimicrobial efficacy. Additionally, since the metabolic pathway that generates ATP has been implicated as a cellular target of silver, ATP levels also provide useful mechanistic information. Bacteria culture and nanocrystalline silver nanodispersions as described for Example 16 were exposed to nanocrystalline silver, and ATP content was measured by luciferase assay (BacTiter GLo assay kit, Promega, Madison, Wis.).

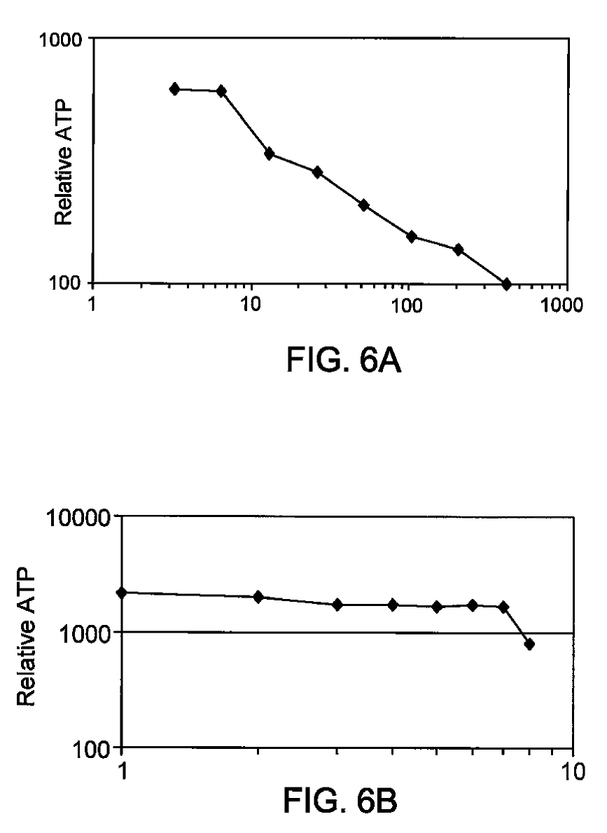

Treatment with nanocrystalline silver decreased the content of ATP in bacterial cells. As shown in FIGS. 6A and 6B, bacterial ATP content was determined by Bac-titer Glo assay after a 20-minute treatment of Ps. aeruginosa with nanocrystalline silver (FIG. 6A) or ciprofloxacin (FIG. 6B). Nanocrystalline silver lowered ATP content of Ps. aeruginosa cells in log-phase growth.

### Example 18

### Comparison of Aerobic and Anaerobic Bactericidal Effects

Surprisingly, in Example 16, the effective concentration of nanocrystalline silver was notably higher against five anaerobic bacterial species (Prop. acnes, C. difficile, Por. gingivalis, B. fragilis, B. thetaiotaomicron) than against other (aerobic or facultative) bacterial species tested. This difference may be attributable to species-dependent differences, culture conditions, the presence/absence of molecular oxygen, and/or the sensitivity of different metabolic pathways to silver. To address the role of oxygen in this discrepancy, MIC values (μg/ml) were obtained under aerobic and anaerobic conditions for nanocrystalline silver and AgNO₃, against facultative anaerobes (i.e. bacteria capable of growing with or without oxygen; Table 14). Nanocrystalline silver is equally effective against aerobic and anaerobic growth of facultative organisms.

TABLE 14

| | |
|---|---|
| Nanocrystalline | |
| silver | AgNO₃ |

Aerobic

| | + | − | + | − |
|---|---|---|---|---|
| Enterobacter cloacae | 11 | 11 | 13 | 25 |
| Serratia marcescens | 11 | 11 | 6 | 13 |
| Klebsiella pneumoniae | 5 | 5 | 3 | 3 |

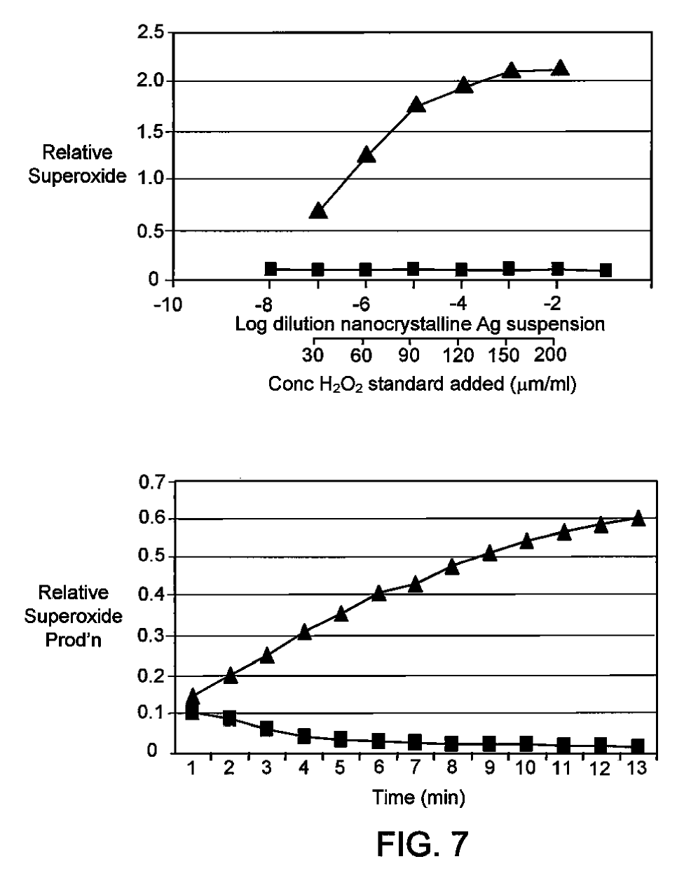

It has been reported that the antibacterial effect of silver ions (Ag+) was dependent upon aeration of the tests samples, and that the antibacterial activity observed under aerobic conditions was inhibited in the presence of agents that scavenge or inactivate reactive oxygen species. Therefore, to further rule out a role of oxygen in antibacterial activity of nanocrystalline silver, we examined whether oxygen radical scavengers impacted the antimicrobial activity. Table 16 shows the MIC values (μg/ml) for nanocrystalline silver and H₂O₂, each tested in the presence and absence of catalase and superoxide dismutase (SOD). Reactive oxygen species hydrogen peroxide and superoxide were neither generated by nanocrystalline silver (FIG. 7) nor did they appear important for the antibacterial activity (Table 15).

TABLE 15

| | |
|---|---|
| E. coli | Ent. cloacae |

SOD

| Catalase | − | + | − | + |
|---|---|---|---|---|
| H₂O₂ | 31 | >500 | 63 | >500 |
| Nanocrystalline silver | 1.4 | 2.8 | 11 | 11 |

Referring to FIG. 7, hydrogen peroxide generation (left) and superoxide generation (right) by nanocrystalline silver (-▪-) were determined in deionized water. Positive controls (-▴-) were reagent-grade hydrogen peroxide and enzymatically generated superoxide, respectively.

Another possible explanation for the difference in MIC is that the anaerobic metabolic pathways used by Prop. acnes, Por. gingivalis, C. difficile Bact. fragilis, and Bact. theaiotaomicron were not as sensitive to nanocrystalline silver as the aerobic metabolisms of the other species tested.

### Example 19

### Treating Biofilm with Silver

Nanocrystalline silver was effective against nascent and established biofilms. Nanocrystalline silver was able to kill Ps. aeruginosa grown in a two-day old biofilm. Nanocrystalline silver concentrations below its MIC could inhibit formation of a biofilm matrix by Ps. aeruginosa.

Bacteria —PAO1 was from Dr. Gerald Pier at BWH. All handling used appropriate biosafety practices.

Bacterial culture—All PAO1 was cultured in either Mueller-Hinton Broth (MHB; Remel, Lenexa, Kans.), or Mueller-Hinton Agar (MHA; BBL) or TSA (PML Microbiological, Mississauga, ON, Canada). Aerobic cultures were done at 32-37° C. Pseudomonas biofilms were grown in Collagen IV or Fibronectin microslides (Discovery Tech Intl, Sarasota, Fla.) using a perfusion flow controller (BioScience Tools, San Diego, Calif.).

Nanocrystalline silver nanodispersion—Nanocrystalline silver was produced by physical vapor deposition using magnetron sputtering, and has a crystallite size of <50 nm. Nanocrystalline silver was added to 0.1 M lactate buffer, pH 4.0 or water, 6% lecithin, and was sonicated and passed through a 0.2-μm cellulose acetate filter (Corning, Corning, N.Y.). The concentration of silver in the final nanodispersion was verified by atomic absorbance spectroscopy.

Biofilm viability staining—Was performed with the BacLight LIVE/DEAD staining reagent (Molecular Probes/Invitrogen, Carlsbad, Calif.) as recommended by the manufacturer.

Results

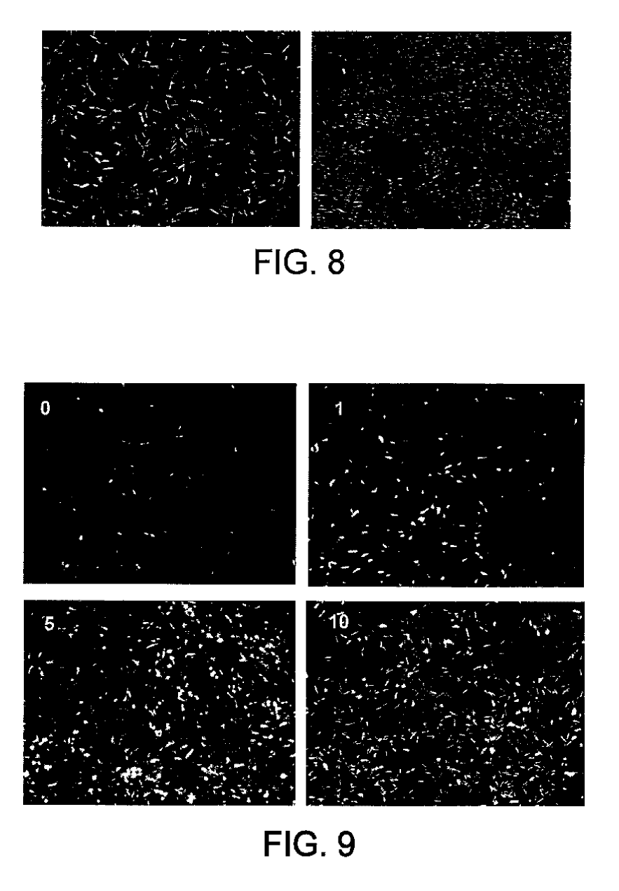

Nanocrystalline silver killed Ps. aeruginosa in established biofilms. As shown in FIG. 8, two-day old Pseudomonas biofilms without (left) and with (right) 2-hour treatment with nanocrystalline silver were stained with a differential staining technique to discern live from dead bacteria. Live cells stain green while dead cells stain red. The cells in the biofilm treated with nanocrystalline silver showed significantly more dead bacteria than the untreated biofilm.

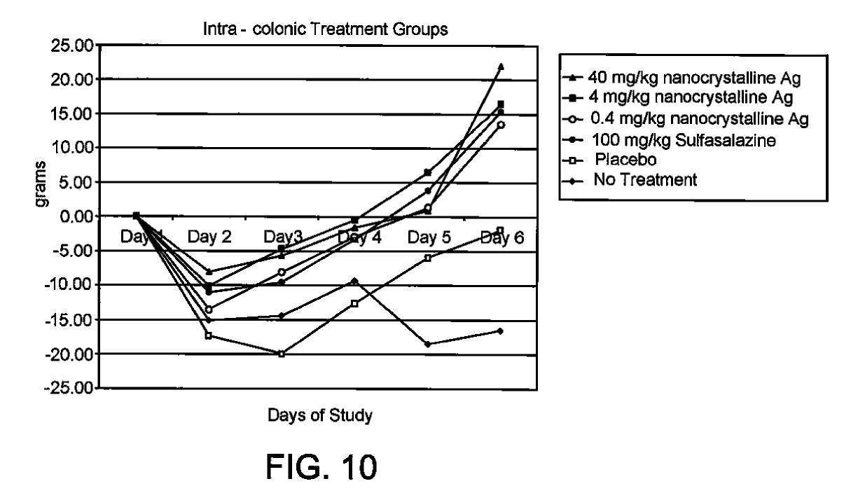

To visualize the effect of nanocrystalline silver on the growth and development of nascent biofilms, Pseudomonas aeruginosa biofilms were grown in a perfusion culture system in the presence of sub-lethal concentrations (e.g., in the presence of 0, 1, 5, or 10 μg/ml (see labels on each panel of FIG. 9)) of nanocrystalline silver. The biofilms were then stained and visualized as described above, live cells stained green while dead cells stained red. The results demonstrate that low concentrations of nanocrystalline silver, below the concentration needed to kill biofilm bacteria, inhibited the production of biofilm carbohydrate matrix (compare the black background of panels marked 1, 5, and 10 to the diffuse green background of the untreated biofilm marked 0). These data suggest that at sub-bactericidal concentrations nanocrystalline silver inhibited the production of biofilm matrix. Without wishing to be bound by theory, it is believed that the matrix is one mechanism of antibiotic resistance among bacterial biofilms. Therefore, biofilms grown in the presence of nanocrystalline silver may be less antibiotic-resistant than biofilms that grow in the absence of nanocrystalline silver.

### Example 20

### In Vitro MBC Assay for Nanocrystalline Silver and Silver Nitrate

Minimal bactericidal concentration (MBC) assay—Overnight-grown MIC plates, prepared as described in Example 16, were evaluated to determine the MIC. Culture was then sampled from wells at and near the MIC using sterile inoculating loops, and transferred to fresh MHA plates to determine if viable bacteria remained. The MHA plates were incubated 18 hours at 37° C. and bacterial growth assessed visually. The MBC was determined as the well in the MIC plate which contained the lowest concentration of silver yielding no bacterial growth on the MHA plate.

Table 16 shows a compilation of direct comparison of MIC to MBC (each indicated in parts silver per million), for nanocrystalline silver as a water-lecithin nanodispersion (Nano Ag), in lactate pH 4.0 (Ag-lactate), and in AgNO₃ solution. The fact that the MBC for each formulation is similar or identical to the corresponding MIC indicates that silver is acting via bactericidal mechanism (i.s. killing the bacteria, as opposed to simply preventing their growth). Abbreviations used for bacterial genera: Staph=Staphylococcus, Ps=Pseudomonas, Strep=Streptococcus, K=Klebsiella, Ser=Serratia, E=Escherichia, Prot=Proteus, C=Citrobacter, B=Burkholderia. Isolates named with only numbers are type strains from the American Type Culture Collection; isolates whose names consist of both letters and numbers are clinical isolates. ni=not interpretable, nt=not tested.

TABLE 16

| | | AgNO₃ | | Nano Ag | | Ag-lactate | |
|---|---|---|---|---|---|---|---|
| Species | Isolate | MIC | MBC | MIC | MBC | MIC | MBC |
| Staph. aureus | NP101 | 6 | 11 | 9 | 19 | 5 | 5 |
| | NP102 | 6 | 6 | 9 | 19 | 5 | 5 |
| | NP103 | 6 | 6 | 19 | 19 | 5 | 10 |
| Ps. aeruginosa | NP201 | 1 | 3 | 5 | 9 | 1 | 3 |
| | NP204 | 1 | 3 | 5 | 9 | 1 | 5 |
| | NP206 | 1 | 1 | 2 | 9 | 0.6 | 0.6 |
| Strep. pyogenes | NP303 | 3 | 10 | 5 | 5 | 3 | 3 |
| | NP304 | 6 | 21 | 9 | 19 | 5 | 6 |
| | NP306 | 6 | 21 | 9 | 9 | 3 | 3 |
| K. pneumoniae | NP401 | 3 | 3 | 9 | 9 | 5 | 5 |
| | NP403 | 6 | 6 | 9 | 9 | 10 | 10 |
| Ser. marcescens | NP402 | 6 | 6 | 9 | 9 | 20 | 10 |
| | NP408 | 6 | 6 | 2 | 1 | 10 | 10 |
| Ser. marcescens | NP402 | 6 | 6 | 19 | 19 | 5 | 5 |
| | NP408 | 11 | 11 | 19 | 19 | 10 | 10 |
| K. pneumoniae | NP403 | 6 | 3 | 9 | 9 | 5 | 5 |
| K. pneumoniae | NP406 | 1 | 1 | 9 | 9 | 1 | 1 |
| E. coli | NP412 | 1 | 1 | 5 | 5 | 1 | 1 |
| Prot. mirabilis | NP405 | 6 | 13 | 19 | 38 | 5 | 10 |
| C. freundii | NP410 | 3 | 3 | 9 | 9 | 3 | 3 |
| B. multivorans | B1 | 3 | 3 | 5 | 10 | 5 | 5 |
| | B2 | 3 | 3 | 5 | 5 | 5 | 5 |
| | B4 | 2 | 3 | 5 | 10 | 3 | 5 |
| B dolosa | A3 | 2 | 3 | 5 | 10 | 3 | 5 |
| | A4 | 2 | 2 | 5 | 5 | 5 | ni |
| Staph epidermidis | 12228 | 3 | 3 | 5 | 5 | 5 | 10 |
| | NP136 | 3 | 3 | 2 | ni | 3 | 5 |
| Staph. aureus | 6538 | 6 | 6 | 5 | 10 | nt | nt |
| Staph. aureus | 700699 | 3 | 6 | 5 | 10 | nt | nt |
| Ps. aeruginosa | 9027 | 1 | ni | 2 | 5 | nt | nt |
| Ps. aeruginosa | 27317 | 2 | 3 | 1 | 2 | nt | nt |
| E. coli | 8739 | 0.4 | 0.4 | 2 | 2 | nt | nt |
| E. coli | 43745 | 1 | 0.4 | 2 | 2 | nt | nt |

### Example 21

### Anti-Microbial Effect of Nanocrystalline Silver Against Mycobacterium tuberculosis

0.1 M lactate buffer, pH 4.0 was prepared by diluting 88% lactic acid (Mallinckrodt) in de-ionized H₂O. The pH was adjusted to 4.0 with 50% aqueous NaOH. Similarly, 0.1 citrate buffer was prepared by dissolving citric acid (OmniPure EM Science) in de-ionized H₂O, Nanocrystalline silver powder was dissolved in lactate buffer or in citrate buffer at a ratio of one mg powder/one ml buffer. The citrate and lactate solutions were each sonicated for 5 minutes then filtered through a 0.2 μm cellulose ester filter (Corning). Portions of the solutions were reserved for determination of dissolved silver content by flame-atomic absorption spectroscopy. Once the silver content was known, the citrate and lactate solutions were then diluted with 0.1 M lactate buffer to a final silver concentration of 400 ppm each.

Silver nitrate solutions were prepared by dissolving 36 milligrams AgNO₃ (Sigma) in 22.8 ml de-ionized H₂O. The solutions were sonicated and filtered as described above, and a portion was reserved for determination of the silver content by flame-atomic absorption spectroscopy. After determination of the silver concentrations, the solution was diluted with de-ionized H₂O to a final silver concentration of 400 ppm.

A silver oxide test solution was prepared by dissolving 13 mg of Ag₂O (Sigma) in 13 ml 0.1M lactate buffer. The solution was sonicated and filtered as described above, and a portion was reserved for determination of actual silver content by flame-atomic absorption spectroscopy. After determination of the silver concentrations, the solution was diluted with de-ionized H₂O to a final silver concentration of 400 ppm.

The nanocrystalline silver, AgNO₃, and Ag₂O formulations were screened against Mycobacterium tuberculosis H37Ra (American Type Culture Collection ATCC 25177) using a calorimetric microdilution broth assay and two-fold dilutions of drug ranging from 0.0098-20 ppm Ag. The antimicrobial activity was evaluated with a calorimetric microdilution broth assay using 96 well microtiter plates. The assay involved diluting each test sample ten-fold in Middlebrook 7H9 broth including 0.2% glycerol and albumin-dextrose-catalase. Twelve serial two-fold dilutions of the initial dilution were prepared in broth and 0.05 mL of each dilution was transferred to appropriate wells in duplicate. Each plate was then inoculated (0.05 mL per well) with culture standardized to 1-2×10⁵ CFU/mL. The assay plates also contained un-inoculated drug and medium controls, and inoculated viability controls. The plates were placed in polyethylene bags and incubated at 36° C. for about six days. Alamar blue was added to each well, the plates were incubated for an additional 18 h and read in an optical plate reader programmed to subtract the absorbance at 600 nm from that at 570 nm to blank out turbidity and absorbance due to oxidized dye. The MIC is reported as the lowest concentration of drug yielding a differential absorbance of zero or less. The color change from blue to red after metabolic reduction of dye is indicative of the concentration at which bacteria growth occurs.

The minimum inhibitory concentrations (MIC) for nanocrystalline silver solutions, Ag₂O solutions, and AgNO₃ solutions were 2.5 μg/ml silver. A positive control using ethambutol resulted in a MIC value of 2.0 μg/ml.

### Example 22

### Bacterial Inhibitory Activity of Nanocrystalline Silver, Ag₂O, and AgNO₃

A minimal inhibitory concentration assay was conducted using a buffered metal-containing solution. A 0.1 M lactate buffer at pH 4.0 was prepared as in Examples 19 and 20. Nanocrystalline silver was dissolved in 0.1 M lactate buffer at pH 4.0 by sonicating a free standing powder of the nanocrystalline silver for five minutes, and filtering the solution through a 0.2-micron filter (Corning, Acton, Mass.) to remove large undissolved crystal aggregates. Buffered lactate solution of Ag₂O and AgNO₃ were also prepared in an analogous manner. The nanocrystalline silver solution, Ag₂O, and AgNO₃ solutions were analyzed by atomic absorption spectrophotometry to determine the dissolved silver content in each solution.

Table 17 shows the MIC values of lactate solutions of nanocrystalline silver, Ag₂O and AgNO₃ against bacterial strains.

TABLE 17

| | P. aerug | | S. aureus | | S. epiderm | | E. coli | | P. aerug. | |
|---|---|---|---|---|---|---|---|---|---|---|
| Silver form | (ATCC) | | (ATCC) | | (ATCC) | | (ATCC) | | (clin.) | |
| Nanocrystalline silver | 0.7-2 | μg/mL | 0.7-2 | μg/mL | 2-6 | μg/mL | 0.5-2 | μg/mL | 1-5 | μg/mL |
| (range for 12 samples) | | | | | | | | | | |
| Ag₂O | 0.8 | μg/mL | 3 | μg/mL | 2 | μg/mL | 0.8 | μg/mL | 2 | μg/mL |
| AgNO₃ | 3 | μg/mL | 11 | μg/mL | 3 | μg/mL | 3 | μg/mL | 3 | μg/mL |

Table 18 shows the MIC values of lactate solutions of nanocrystalline silver, Ag₂O and AgNO₃ against bacterial strains.

TABLE 18

| | S. aureus | | S. pyog | | S. pyog | | S. pneumo | | S. pneumo | |
|---|---|---|---|---|---|---|---|---|---|---|
| Silver form | (clin.) | | (clin. 303) | | (clin. 304) | | (clin 314) | | (clin 315) | |
| Nanocrystalline silver | 8-15 | μg/mL | 2-6 | μg/mL | 3-6 | μg/mL | 5-10 | μg/mL | 3-8 | μg/mL |
| (range for 12 | | | | | | | | | | |
| samples) | | | | | | | | | | |
| Ag₂O | 6 | μg/mL | 2 | μg/mL | 3 | μg/mL | 3 | μg/mL | 2 | μg/mL |
| AgNO₃ | 5 | μg/mL | 3 | μg/mL | 5 | μg/mL | 11 | μg/mL | 21 | μg/mL |

### Example 23

### Bacterial Inhibitory Activity for Different Silver Formulations Against Bacterial Strains

Lactate solutions of nanocrystalline silver were prepared as previously described. Alternatively, nanocrystalline silver powder or AgNO₃ was incorporated into a water-lecithin or water-PVA nanodispersions. The total silver content was determined by atomic absorption spectroscopy.

Table 19 shows MIC values (in ppm) obtained with nine different formulations of nanocrystalline silver or AgNO₃, against P. aeruginosa isolate NP212.

TABLE 19

| | Formulation type | | | | | | | | |
|---|---|---|---|---|---|---|---|---|---|
| | Lactate | | | Water/PVA | | | Water/Lecithin | | |
| | Silver sonrce | | | | | | | | |
| | Nano | | | Nano | | | Nano | | |
| | Ag | AgNO₃ | none | Ag | AgNO₃ | none | Ag | AgNO₃ | none |
| NP212 MIC | 2 | 2 | n.i. | 23 | 3 | n.i. | 8 | 3 | n.i. |

n.i. = no inhibition was observed.

Table 20. MIC values (in ppm) obtained with nine different formulations of nanocrystalline silver or AgNO₃, against P. aeruginosa isolates NP213 and NP 214, and against Staph aureus isolates NP101, NP102, and NP103.

TABLE 20

| | Formulation type | | | | | | | | |
|---|---|---|---|---|---|---|---|---|---|
| | Lactate | | | Water/PVA | | | Water/Lecithin | | |
| | Silver sonrce | | | | | | | | |
| | Nano | | | Nano | | | Nano | | |
| | Ag | AgNO₃ | none | Ag | AgNO₃ | none | Ag | AgNO₃ | none |
| NP213 MIC | 1 | 1 | n.i. | 6 | 1 | n.i. | 2 | 1 | n.i. |
| NP214 MIC | 2 | 1 | n.i. | 6 | 3 | n.i. | 2 | 1 | n.i. |
| NP101 MIC | 9 | 9 | n.i. | 46 | 11 | n.i. | 15 | 11 | n.i. |
| NP102 MIC | 9 | 9 | n.i. | 46 | 11 | n.i. | 15 | 11 | n.i. |
| NP103 MIC | 9 | 9 | n.i. | 46 | 11 | n.i. | 15 | 11 | n.i. |

n.i. = no inhibition was observed.

Table 21 shows MIC values (in ppm) obtained with nine formulations of nanocrystalline Ag or AgNO₃, against isolates of: P. aeruginosa (NP 201, NP202), Strep. pyogenes (NP303, NP304, NP306), Strep. pneumoniae (NP316), Klebsiella pneumoniae (NP401, NP403), Serratia marcescens (NP402, 408), Burkholderia dolosa (SLC6-3, SLC6-4), Burk multivorans (BBM2, BBM4), or Candida albicans (NP501, NP502)

TABLE 21

| | Formulation type | | | | | | | | |
|---|---|---|---|---|---|---|---|---|---|
| | Lactate | | | Water/PVA | | | Water/Lecithin | | |
| | Silver source | | | | | | | | |
| | Nano | | | Nano | | | Nano | | |
| | Ag | AgNO₃ | none | Ag | AgNO₃ | none | Ag | AgNO₃ | none |
| NP201 MIC | 2 | 2 | (200x) | 40 | 2 | n.i. | 6 | 2 | n.i. |
| NP202 MIC | 2 | 1 | n.i. | 20 | 2 | n.i. | 6 | 2 | n.i. |
| NP303 MIC | n.d. | n.d. | n.d. | 10 | 1 | n.i. | n.d. | n.d. | n.d. |
| NP304 MIC | n.d. | n.d. | n.d. | 20 | 1 | n.i. | 3 | 1 | n.i. |
| NP306 MIC | 1 | n.d. | (200x) | 10 | 1 | n.i. | 3 | 2 | n.i. |
| NP316 MIC | 1 | 1 | (100x) | 20 | 1 | n.i. | 3 | 1 | n.i. |
| NP401 MIC | 4 | 2 | n.i. | 40 | 2 | n.i. | 6 | 2 | n.i. |
| NP403 MIC | 1 | 0.6 | n.i. | 20 | 1 | n.i. | 13 | 1 | n.i. |
| NP402 MIC | 8 | 9 | n.i. | 81 | 5 | n.i. | 13 | 5 | n.i. |
| NP408 MIC | 8 | 9 | n.i. | 81 | 10 | n.i. | 13 | 9 | n.i. |
| SLC6-3 MIC | 2 | 1 | (200x) | 20 | 2 | n.i. | 6 | 2 | n.i. |
| SLC6-4 MIC | 4 | 2 | (100x) | 20 | 2 | n.i. | 6 | 2 | n.i. |
| BBM2 MIC | 2 | 2 | n.i. | 20 | 2 | n.i. | 6 | 2 | n.i. |
| BBM4 MIC | 2 | 2 | n.i. | 40 | 2 | n.i. | 6 | 2 | n.i. |
| NP501 MIC | 16 | 36 | n.i. | 133 | 38 | n.i. | 103 | 18 | n.i. |
| NP502 MIC | 9 | 9 | n.i. | 46 | 11 | n.i. | 15 | 11 | n.i. |

n.i. = no inhibition was observed.
n.d. = not determined.
100x and 200x indicate respectively that the lactate control did inhibit growth, but required 100 and 200-fold more lactate buffer than silver-containing formulations.

Table 22 shows MIC values (in ppm) obtained with nine different formulations of nanocrystalline silver or AgNO₃, against strains/isolates of: Aspergillis niger (ATCC16404), A. fumigatis (NP512, NP513), Proteus mirabilis (NP405), Citrobacter freundii (NP410), Strep pyogenes (NP307, NP308, NP309, NP310), Eschericha coli (ATCC700417, ATCC700928)

TABLE 22

| | Formulation type | | | | | | | | |
|---|---|---|---|---|---|---|---|---|---|
| | Lactate | | | Water/PVA | | | Water/Lecithin | | |
| | Silver source | | | | | | | | |
| | Nano | | | Nano | | | Nano | | |
| | Ag | AgNO₃ | none | Ag | AgNO₃ | none | Ag | AgNO₃ | none |
| 16404 MIC | 16 | 7 | n.i. | 130 | 9 | n.i. | 26 | 18 | n.i. |
| NP405 MIC | 4 | 2 | n.i. | 65 | 5 | n.i. | 13 | 5 | n.i. |
| NP410 MIC | 4 | 4 | n.i. | 32 | 5 | n.i. | 13 | 5 | n.i. |
| NP307 MIC | 1 | 0.5 | (500X) | 32 | 1 | n.i. | 2 | 1 | n.i. |
| NP308 MIC | 1 | 0.5 | (500x) | 16 | 1 | n.i. | 2 | 1 | n.i. |
| NP309 MIC | <0.3 | <0.3 | (2000X) | 16 | 0.6 | (1000x) | 0.9 | 0.6 | (1000x) |
| NP310 MIC | 1 | 1 | (500x) | 16 | 1 | n.i. | 2 | 1 | n.i. |
| NP512 MIC | 32 | 17 | n.i. | 260 | 18 | n.i. | 13 | 18 | n.i. |
| NP513 MIC | 16 | 17 | n.i. | 260 | 18 | n.i. | 26 | 18 | n.i. |
| 700417 MIC | 64 | n.d. | n.i. | 130 | 9 | n.i. | 26 | 9 | n.i. |
| 700928 MIC | 1 | 0.5 | n.i. | 32 | 1 | n.i. | 3 | 1 | n.i. |

n.i. = no inhibition was observed.
n.d. = not determined.
500x, 1000x, and 2000x indicate respectively that the placebo control did inhibit growth, but required 500-, 1000-, and 2000-fold more buffer than silver-containing formulations.

Table 23 shows MIC values (in ppm) obtained with nine different formulations of nanocrystalline Ag or AgNO₃, against Shigella flexneri Type strain ATCC 12022.

TABLE 23

| | Formulation type | | | | | | | | |
|---|---|---|---|---|---|---|---|---|---|
| | Lactate | | | Water/PVA | | | Water/Lecithin | | |
| | Silver source | | | | | | | | |
| | Nano | | | Nano | | | Nano | | |
| | Ag | AgNO₃ | none | Ag | AgNO₃ | none | Ag | AgNO₃ | none |
| 12022 MIC | 2 | 2 | n.i. | 9 | 1 | n.i. | 3 | 2 | n.i. |

n.i. = no inhibition was observed.

Table 24 shows MIC values (in ppm) obtained with nine different formulations of nanocrystalline Ag or AgNO₃, against strains and isolates of Pseudomonas aeruginosa (6294 and 6077), Streptococcus agalactiae (ATCC 12403), Strep. mutans (ATCC 33402), Strep. mitis (ATCC 15914), and Strep. bovis (ATCC 35034)

TABLE 24

| | Formulation type | | | | | | | | |
|---|---|---|---|---|---|---|---|---|---|
| | Lactate | | | Water/PVA | | | Water/Lecithin | | |
| | Silver source | | | | | | | | |
| | Nano | | | Nano | | | Nano | | |
| | Ag | AgNO₃ | none | Ag | AgNO₃ | none | Ag | AgNO₃ | none |
| 6294 MIC | 2 | 2 | (200x) | 13 | 2 | n.i. | 5 | 2 | n.i. |
| 6077 MIC | 2 | 2 | (200x) | 26 | 2 | n.i. | 9 | 2 | n.i. |
| 12403 MIC | 2 | 2 | (100x) | 13 | 2 | n.i. | 5 | 2 | n.i. |
| 33402 MIC | <0.2 | <0.3 | (2000x) | 3 | 0.5 | n.i. | 2 | 0.5 | n.i. |
| 15914 MIC | 4 | 4 | (64x) | 13 | 4 | n.i. | 9 | 4 | n.i. |
| 35034 MIC | 2 | 1 | (100x) | 13 | 2 | n.i. | 5 | 2 | n.i. |

n.i. = no inhibition was observed.
64x, 100x, 200x, and 2000x indicate respectively that the placebo control did inhibit growth, but required 64-, 100-, 200- and 2000-fold more buffer than silver-containing formulations.

Table 25 shows MIC values (in ppm) obtained with nine different formulations of nanocrystalline silver or AgNO₃, against strains and isolates of Staphylococcus hominis (ATCC 27844), Staph. saprophyticus (ATCC 15305), Staph. warneri (ATCC 27836), and Staph. haemolyticus (ATCC 29970). Pseudomonas stutzeri (ATCC 11607), Morganella morganii (ATCC 29853), Enterobacter cloacae (NP404), Staph. epidermidis (ATCC 12228)

TABLE 25

| | Formulation type | | | | | | | | |
|---|---|---|---|---|---|---|---|---|---|
| | Lactate | | | Water/PVA | | | Water/Lecithin | | |
| | Silver source | | | | | | | | |
| | Nano | | | Nano | | | Nano | | |
| | Ag | AgNO₃ | none | Ag | AgNO₃ | none | Ag | AgNO₃ | none |
| 27844 MIC | 4 | 4 | (100x) | 26 | 4 | n.i. | 9 | 4 | n.i. |
| 15305 MIC | 8 | 9 | (32x) | 53 | 9 | n.i. | 19 | 9 | n.i. |
| 27836 MIC | 4 | 4 | (64x) | 53 | 4 | n.i. | 19 | 4 | n.i. |
| 29970 MIC | 8 | 9 | (32x) | 53 | 9 | n.i. | 19 | 9 | n.i. |
| 11607 MIC | 4 | 4 | (100x) | 13 | 4 | n.i. | 5 | 5 | (100x) |
| 29853 MIC | 8 | 9 | (64x) | 53 | 4 | n.i. | 19 | 9 | n.i. |
| NP404 MIC | 4 | 2 | (200x) | 26 | 4 | n.i. | 9 | 4 | n.i. |
| 12228 MIC | 1 | 1 | (500x) | 13 | 2 | n.i. | 9 | 2 | n.i. |

n.i. = no inhibition was observed.
32x, 64x, 100x, 200x, and 500x indicate respectively that the placebo control did inhibit growth, but required 32-, 64-, 100-, 200-, and 500-fold more buffer than silver-containing formulations.

Table 26 shows MIC values (in ppm) obtained with nine different formulations of nanocrystalline Ag, or AgNO₃, against strains and isolates of Proteus vulgaris (ATCC 12454), Trichophyton mentagrophytes (ATCC 11481), Trich. Rubrum (ATCC 10218), Cryptococcus neoformans (NP516), Aspergillis niger (ATCC 16404), Aspergillis fumigatis (ATCC 208995, 90906), Propionibacterium acnes (ATCC 11878, 11827), Bacteroides thetaiotaomicron (ATCC 29741), Porphyromonas gingivalis (ATCC 33277), Bacteroides fragilis (ATCC 25285)

TABLE 26

| | Formulation type | | | | | | | | |
|---|---|---|---|---|---|---|---|---|---|
| | Lactate | | | Water/PVA | | | Water/Lecithin | | |
| | Silver source | | | | | | | | |
| | Nano | | | Nano | | | Nano | | |
| | Ag | AgNO₃ | none | Ag | AgNO₃ | none | Ag | AgNO₃ | none |
| 12454 MIC | 1 | 1 | (500x) | 26 | 4 | n.i. | 7 | 2 | n.i. |
| 11481 MIC | 31 | 34 | (8x) | 210 | 17 | n.i. | 27 | 68 | n.i. |
| 10218 MIC | 31 | 34 | (16x) | 210 | 34 | n.i. | 54 | 68 | n.i. |
| NP516 MIC | 31 | 34 | n.i. | 210 | 34 | n.i. | 54 | 34 | n.i. |
| 16404 MIC | 16 | 17 | n.i. | 105 | 17 | n.i. | 27 | 17 | n.i. |
| 208995 MIC | 31 | 34 | n.i. | 210 | 34 | n.i. | 107 | 34 | n.i. |
| 11828 MIC | 249 | 273 | n.i. | >420 | >545 | n.i. | 54 | >136 | n.i. |
| 11827 MIC | 249 | 273 | n.i. | >420 | >545 | n.i. | 54 | >136 | n.i. |
| 29741 MIC | 124 | 137 | n.i. | 53 | 17 | n.i. | 54 | 136 | n.i. |
| 33277 MIC | 31 | 34 | n.i. | 105 | 34 | n.i. | 54 | 34 | n.i. |
| 25285 MIC | 31 | 34 | n.i. | 105 | 34 | n.i. | 54 | 34 | n.i. |
| 90906 MIC | 31 | 34 | n.i. | 105 | 34 | n.i. | 54 | 34 | n.i. |

n.i. = no inhibition was observed.
8x, 16x, and 500x indicate respectively that the placebo control did inhibit growth, but required 8-, 16-, and 500-fold more buffer than silver-containing formulations.

Table 27 shows macrodilution MIC values (in ppm) obtained with four different formulations of nanocrystalline silver or AgNO₃, against Helicobacter pylori Type strain ATCC 43504

TABLE 27

| | Formulation type | | | | |
|---|---|---|---|---|---|
| | Lactate | | Lecithin | | |
| | Silver source | | | | |
| | Nano Ag | AgNO₃ | Nano Ag | AgNO₃ | none |
| 43504 MIC trial 1 | 1 | 9 | 7 | 5 | n.d. |
| 43504 MIC trial 2 | | | 29 | 36 | n.i. |

n.i. = no inhibition was observed.
n.d. = not determined.

All silver-containing formulations inhibited bacterial growth. In lactate buffer the nanocrystalline silver and AgNO₃ behaved similarly. In water nanodispersions, the activity and/or the availability of nanocrystalline silver was reduced, while that of AgNO₃ was unaffected. Nanocrystalline silver was less active when dispersed in PVA, relative to lecithin nanodispersions and lactate formulations. Most assays showed nanocrystalline silver to have a similar (e.g., equal) activity to that of AgNO₃ (e.g., within 10-fold). Where nanocrystalline silver and AgNO₃ are not within 2 fold, they were within 40-fold, indicating that activities of nanocrystalline silver and AgNO₃ were likely very similar.

### Example 24

### Acute Toxicity Study of Nanocrystalline Silver in Mice

The mice were BALB/c mice, weighing 17-20 g (Charles River Lab, Wilmington, Mass.). The mice were acclimatized for 203 days and groups of four animals were placed in metabolic cages and maintained under standardized condition with 12 hours light and dark cycles. The animals were allowed rodent meal and water ad libitum. Different doses of nanocrystalline silver powder in 0.5% carboxymethylcellose were orally gavaged once daily for one day or five days. Animals were examined daily and the mortality rates recorded. Urine and feces were collected daily. The animals were sacrificed after five days of treatment. Liver, lungs, brains, spleens, hearts, kidneys as well as skin and carcass samples were collected and stored frozen for analysis of silver using atomic spectroscopy. For the analysis of silver in feces and carcass samples in which higher levels of silver were detected, Flame atomic absorption spectroscopy (FAAS) was used. All other samples were analyzed using Graphite Furnace Atomic Spectroscopy (GRAAS).

Referring to Table 28, acute toxicity of nanocrystalline silver was assessed daily in mice by administering a single dose of nanocrystalline silver in 0.5% by weight carboxymethylcellulose and monitoring the survival rate of mice.

TABLE 28

| Single oral dose | Dead/Treated | % mortality |
|---|---|---|
| 1200 mg/kg | 4/4 | 100 |
| 700 mg/kg | 3/5 | 75 |
| 600 mg/kg | 1/4 | 25 |
| 350 mg/kg | 0/4 | 0 |
| 150 mg/kg | 0/4 | 0 |

The oral dosing of nanocrystalline silver at 1200 mg/kg and 700 mg/kg was lethal to mice, while doses below 600 mg/kg were well tolerated. The analysis of tissue for silvered showed that the predominant uptake was in the spleen and liver. Analysis of urine and feces indicated that minor amounts of silver were excreted via the urinary system (e.g., μg levels) and the majority of material administered was excreted in the feces (e.g., mg levels).

### Example 25

### Rat Model for Treatment of IBD Using Nanocrystalline Silver

Animals: Male Sprague-Dawley rats, weighing approximately 250-300 gm were purchased from Charles River Laboratories (Raleigh, N.C.). The animals were housed under standard conditions with free access to food and water. They were acclimatized for 2-3 days before commencing the experiments. The study protocol was approved by the Institutional Animal Care and Use Committees (IACUC).

Induction of ulcerative colitis: Colitis was produced according to published methods. Animals were anesthetized by intraperitoneal injection of 75 mg/kg of ketamine plus 5 mg/kg of xylazine. A single intracolonic injection of 120 mg/ml of dinitrobenzenesulfonic acid (DNBS) (Sigma Chemicals, St. Louis, Mo.) in 250 micro liter of 50% ethanol was administered through a Tom Cat catheter (Kendall Sovereign, Mansfield, Mass.) with a closed-end tip, connected to a 1 ml syringe and placed 7 cm proximal to the anus. Lubricant was applied to the tip of the catheter before injection, to protect from mucosal injury. After instillation of DNBS, 0.5 ml of air was insufflated and the catheter was removed.

Treatment: Nanodispersions with different concentrations (40 mg/kg, 4 mg/kg, 0.4 mg/kg) of nanocrystalline silver were prepared by adding desired amounts of nanocrystalline silver and 5.7% polyvinyl alcohol (PVA) (Alfa Aesar, Ward Hill, Mass.) in water then dispersed using a probe type sonicator (Model UP400S, Ultraschallprozessor, GmbH). Sulfasalazine (Sigma Chemicals, St Louis, Mo.), at a concentration of 120 mg/ml for intracolonic treatment and 30 mg/ml for oral treatment was used as the reference drug in all the experiments. Sulfasalazine was first dissolved in 0.1M NaOH at a concentration about 30-fold higher than the required final concentration, and then suspended in 1.5% PVA for oral dosing and 5.7% PVA for intracolonic dosing. The suspension was sonicated using an Ultrasonic model 150D (VWR) for 30 minutes.

In the intracolonic treatment groups, 250 μl of placebo (5.7% PVA), nanodispersions of nanocrystalline silver in 5.7% PVA (40, 4, 0.4 mg/kg) or sulfasalazine (100 mg/kg) was administered intracolonically using a Tom-cat catheter as described for DNBS administration. In the oral treatment groups, 1 ml of 1.5% PVA, nanocrystalline silver nano-dispersions (40, 4, 0.4 mg/kg) or 100 mg/kg of sulfasalazine suspension was administered by oral gavage. One group was not treated and served as no treatment group. Treatment began one day after administration of DNBS.

Macroscopic Observations: Body weight was recorded daily and the animals were examined for their physical appearance. After five days of treatment, animals were sacrificed. The colon was excised between the ileocecal junction and rectum, and opened longitudinally. After scoring of the consistency of the stool the colon was rinsed in normal saline, examined and thickness of the colonic wall and number and size of ulcers were scored. Stool consistency was graded on a scale of 0-3: 0=normal stool, well formed pellets rigid as normal; 1=loose stool, formed pellet with moisture, soft stool keeping in stool shape; 2=loose stool, stools that have abnormal form with much moisture, softer shapeless stool; 3=watery or bloody diarrhea.

Thickness of colonic wall was graded on a scale of 0-4: 0=normal, no thickness; 1=mild thickness in lower portion of the colon; 2=moderate thickness in localized area; 3=moderate thickness extending to the central portion of the colon; 4=severe thickness in the whole colon. Colonic ulceration was graded on a scale of 0-10: 0=no ulcers; 1=one small ulcer less than 2-3 mm; 2=one large ulcer more than 2-3 mm; 3=two large ulcerations more than 2-3 mm in diameter; 4=two or more sites of ulcerations extending 1 cm along the length; 5-10=when an area of ulceration extended more than 1 cm along the length of the colon, the score increases by 1 for each additional cm of involvement. The total IBD score, obtained by the sum of the scores of colonic ulcer, colon thickness and stool consistency for each animal, was calculated for each treatment group.

Histopathology: A piece of colonic tissue at 2-4 cm proximal to the anus was excised and fixed in 10% formalin and the sections were stained with hematoxylin and eosin. The remaining length of the colonic tissues was stored at −80° C. for subsequent biochemical assays. The histology slides were examined by a veterinary pathologist who was blinded to the treatment groups. The extent of microscopic mucosal damage of the colon was scored on scale of 0-5 as described with slight modification: 0=normal histological appearance; 1=histological damage limited to surface epithelium, mild infiltration of the submucosa; 2=focal ulceration and cell disruption limited to mucosa, abnormal appearance of colonic wall, mild infiltration of inflammatory cells; 3=focal, transmural inflammation and ulceration, with mild to moderate infiltration of inflammatory cells; 4=Extensive transmural ulceration and inflammation bordered by areas of normal mucosa, moderate infiltration of inflammatory cells; 5=extensive transmural ulceration and inflammation involving entire section from epithelium to serosa, severe infiltration of inflammatory cells.

Immunohistochemistry: A Universal Streptavidin/Biotin detection system (Thermo Shandon, Pittsburgh, Pa.) was used for immunohistochemical staining of formalin fixed, paraffin embedded biopsies of the colon. After dewaxing, and hydration, sections were treated with protein blocking agent to reduce non-specific binding of antibodies. The tissues were then incubated with rabbit anti-rat TNF-α, or mouse anti-rat IL-12 p70 antibodies (Biosource International, Inc, Camarillo, Calif.); rabbit anti-rat IL-1β (Serotec, Raleigh, N.C.) or rabbit anti-rat MMP-9 antibody (Chemicon International, Telecuma, Calif.) in 1% bovine serum albumin in phosphate buffered saline. Incubations with secondary antibodies, streptavidin/biotin, and chromogenic detection of peroxidase were performed according to the manufacturer's protocol. Sections were counterstained, dehydrated, mounted and examined. The intensity of the immuno-peroxidase staining was graded as described previously. Expression of cytokines was graded as: 0, no staining in the section; 1, less than 25% of the section; 2, 25-50% of the section; 3, 50-75% of the section; 4, >75% of the section with significant staining.

Gelatin Zymography Activities of MMP-9 and MMP-2 in colonic tissue homogenates were determined by gelatin zymography as described previously. Briefly, colonic homogenates were mixed with equal volume of zymograph sample buffer. Samples were loaded on a 10% SDS-polyacrylamide gel containing 0.1% gelatin. After electrophoresis, the SDS was removed from the gel by incubation with renaturing buffer containing 2% Triton-X-100 for 1 hour. The gel was incubated overnight in developing buffer containing 50 mM Tris-HCl, pH 7.8, 5 mM CaCl₂, 0.2 M NaCl, 0.02% Brij-35. The gel was subsequently stained with Coumassie blue for 30 minutes and destained with destaining solution containing 8% acetic acid and 4% methanol. Gelatinolytic activity was determined as transparent bands against the blue background of stained gelatin. MMPs were identified by their molecular weight compared with the standards. Absence of a transparent band on the gel was evidenced as no MMP-9 or MMP-2 activity of the sample tested.

Statistical Analysis: The results are expressed as the Mean±SE. The statistical significance in the mean total IBD score, histopathological inflammation score, and the immunohistochemical staining of cytokines or MMP-9 was determined using Tukey-Kramer's multiple comparison tests following one-way analysis of variance (ANOVA). The data were calculated with the aid of Instat Graph Pad Software. Differences with a P value less than 0.05 were considered significant.

Results:

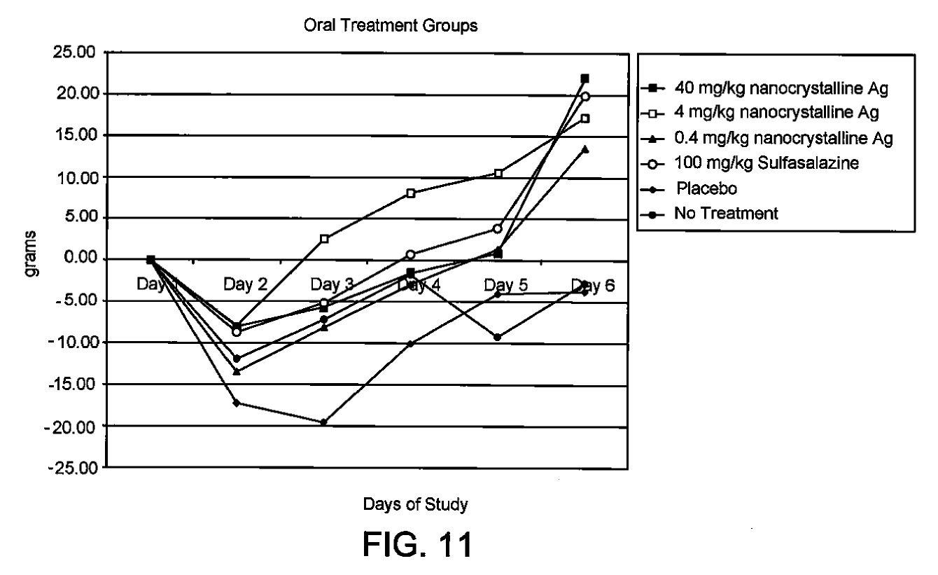

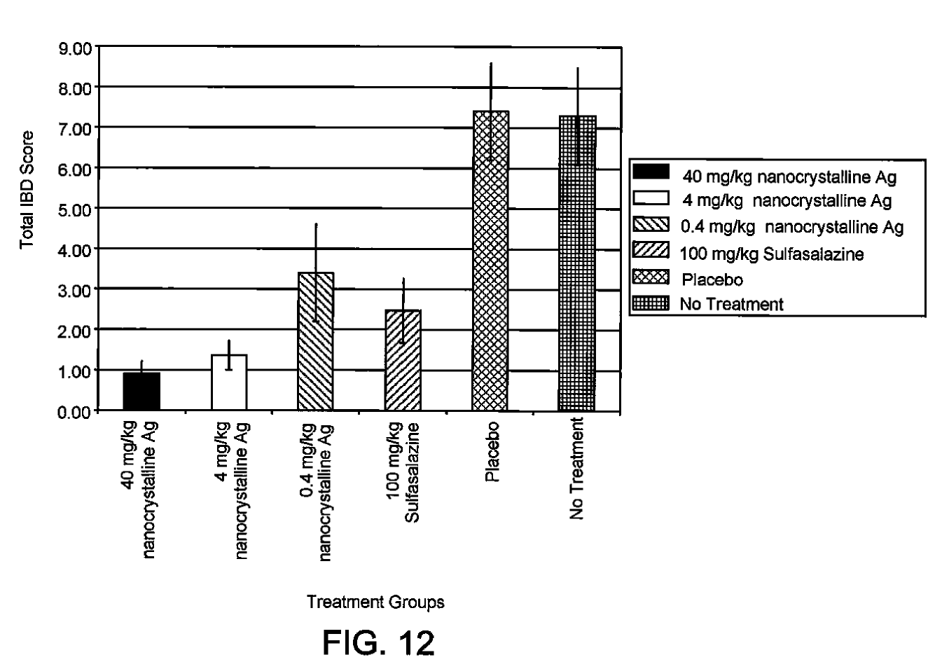

Effects of treatment with nanocrystalline silver and sulfasalazine on DNBS-induced colitis: Reduction of body weight was observed in the majority of animals in all groups one day after induction of ulcerative colitis (FIGS. 10, 11). The animals in groups treated with nanocrystalline silver and sulfasalazine started gaining body weight after one day of treatment, whereas body weight of the animals in the no treatment group or placebo group did not regain original body weight by the end of the study.

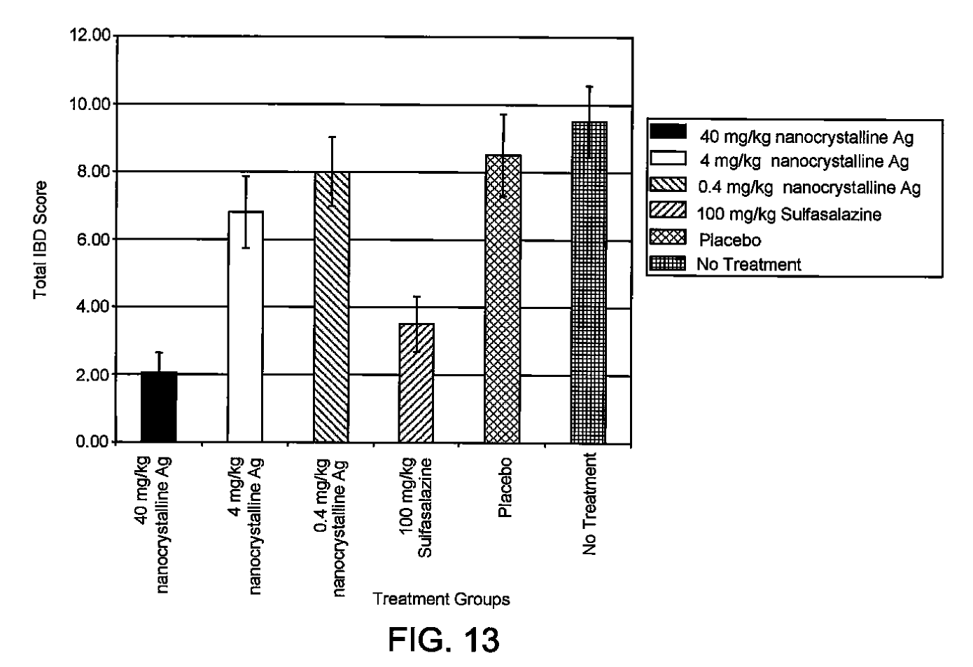

Intracolonic treatment of nanocrystalline silver nanodispersion with 40 mg/kg and 4 mg/kg, but not 0.4 mg/kg, demonstrated a significant reduction of total IBD score after five days of treatment, compared to the placebo and no treatment groups (FIG. 12). The animals treated with placebo demonstrated that PVA alone was unable to produce reduction of the IBD score, compared to untreated group.

Significant reduction of IBD score was observed after five days of intracolonic treatment of 100 mg/kg of sulfasalazine.

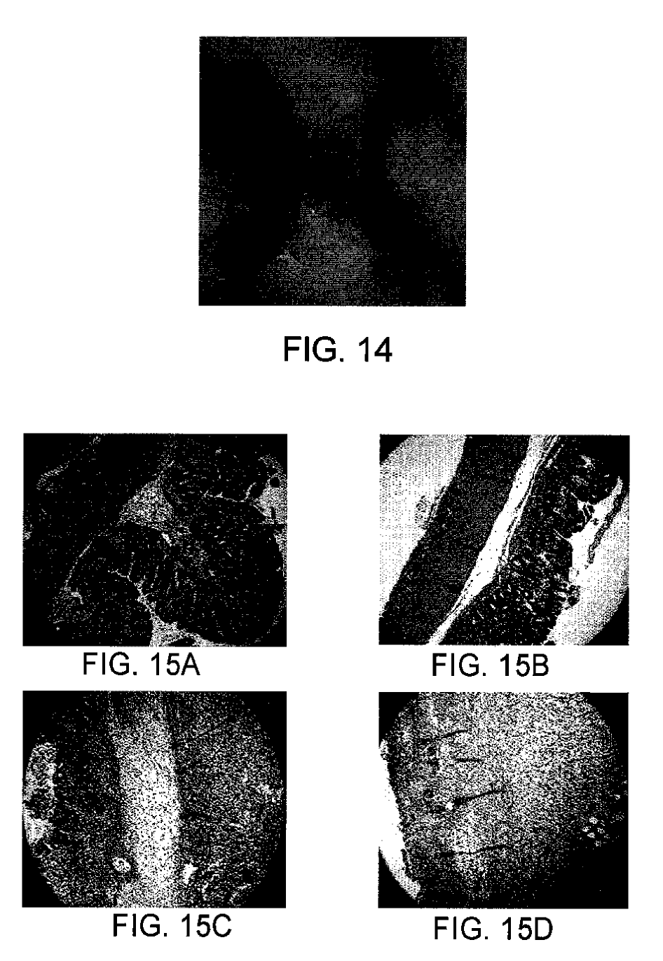

Oral treatment of 40 mg/kg of nanocrystalline silver, but not 4 mg/kg and 0.4 mg/kg, for five days demonstrated a significant reduction of the total IBD score compared to the placebo and no treatment groups (FIG. 13). A significant reduction of the IBD score was observed after five days of oral treatment with 100 mg/kg of sulfasalazine.

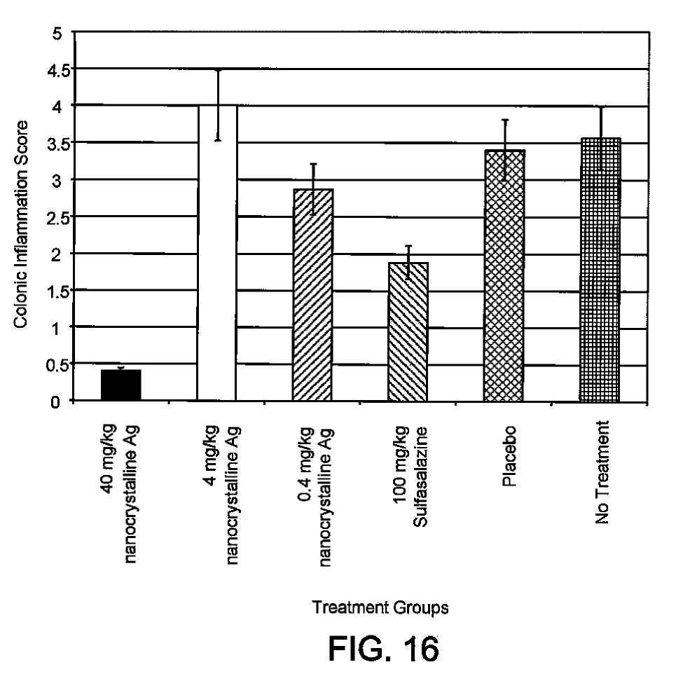

Referring to FIG. 14, colons non-treated and treated with nanocrystalline silver were visually different upon inspection. The non-treated colons were ulcerated and swollen, while the treated colons showed decreased inflammation and appeared healthier.

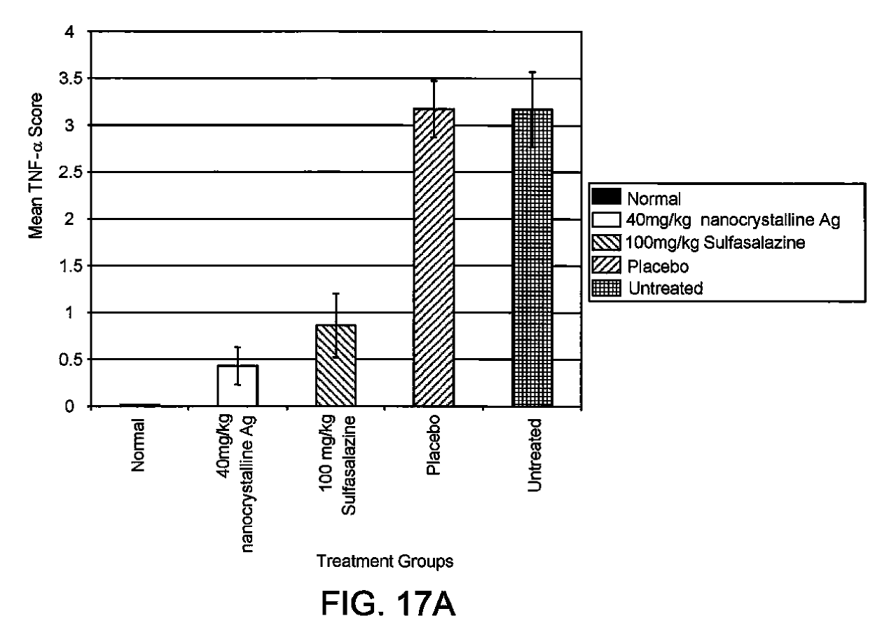

Histopathological observation: Colonic sections from the oral treatment groups were microscopically examined. Generally, colonic damage consisted of transmural ulceration extending through the serosal surface, with presence of edematous muscularis mucosa and submucosa, infiltration of neutrophils, lymphocytes (FIG. 15A-15D). FIG. 15A-15D are representative photographs showing sections of hematoxylin and eosin stained colon tissues after treatment with nanocrystalline silver. 15A was treated with 40 mg/kg nanocrystalline silver, 15B was treated with 100 mg/kg sulfasalazine, 15C was treated with placebo, and 15D was untreated. There was healed mucosa and few residual inflammatory infiltrate in the sections of rat treated with nanocrystalline silver and sulfasalazine (16A, 16B), while there was extensive ulceration and inflammatory infiltrate in the sections of placebo treated and untreated rats (16C, 16D)

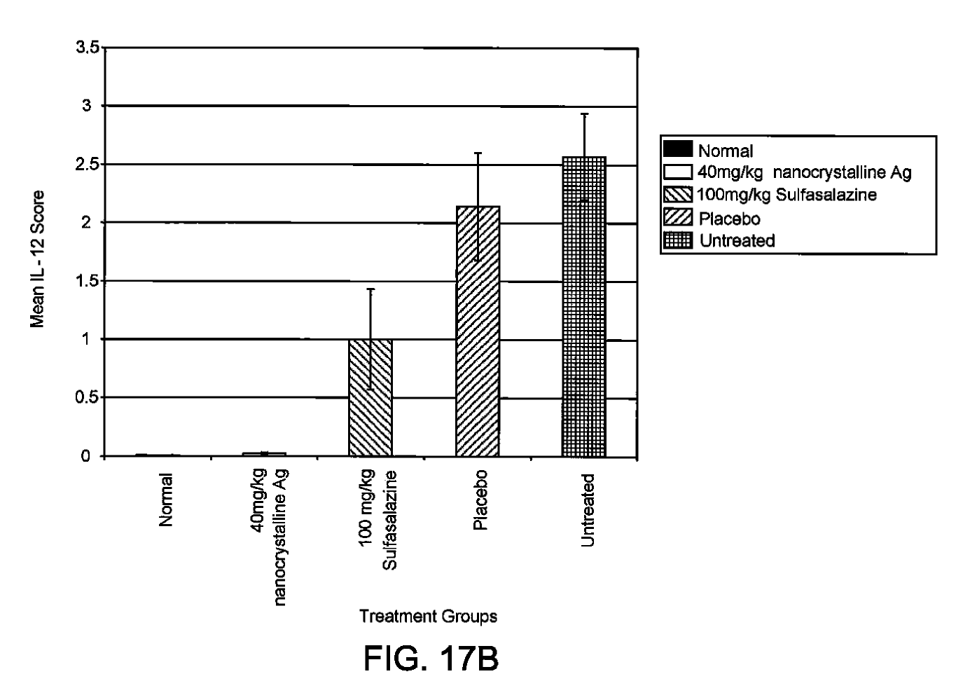

Consistent with the observed effect of oral nanocrystalline silver on the total IBD score, the highest dose (40 mg/kg), but not the lower doses (4 mg/kg and 0.4 mg/kg), significantly reduced the histopathological inflammation score compared to placebo and untreated groups (FIG. 16). Placebo treatment was unable to reduce histopathological inflammation in the colon, compared to untreated group. Sulfasalazine significantly reduced the histopathological inflammation compared to placebo and untreated groups, but 40 mg/kg nanocrystalline silver showed significantly greater reduction than sulfasalazine treatment.

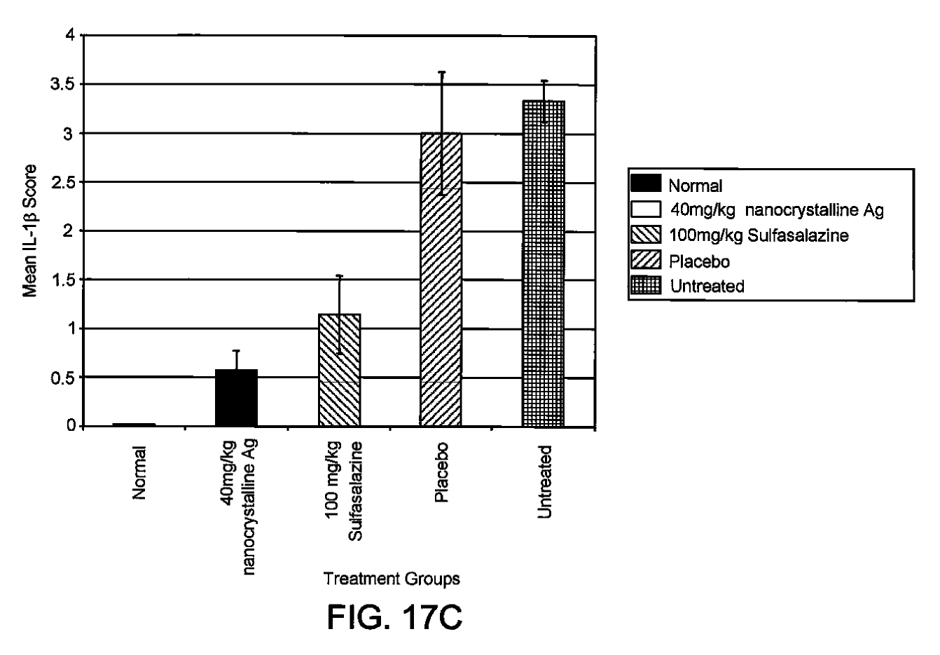

Effect of nanocrystalline silver on modulation of inflammatory cytokines: Protein expression of inflammatory cytokines TNF-α, IL-12 and IL-1β was examined by immunohistochemical staining of the colonic tissues. TNF-α, IL-12B, and IL-1β were up regulated in the colon after DNBS-induced colitis in rat (FIGS. 17A, 17B, 17C). Nanocrystalline silver markedly reduced the expression of these cytokines. Interestingly, sulfasalazine significantly reduced the expression of TNF-α and IL-1β, but not IL-12.

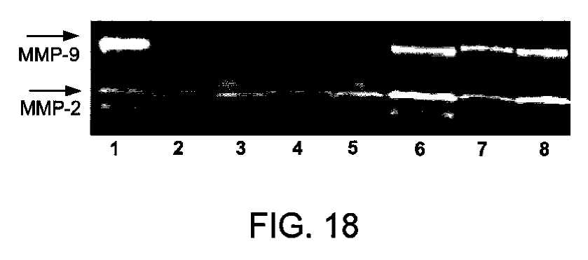

Effect of nanocrystalline silver on MMP-9 and MMP-2: DNBS induction of colitis resulted in the appearance of MMP-9 activity. FIG. 18 shows a representative picture of a gelatin zymography using colonic homogenates of DNBS-induced colitis after oral treatment with nanocrystalline silver and sulfasalazine. Lane 1, standard; 2, normal colon; 3, sulfasalazine 100 mg/kg; 4, nanocrystalline silver 40 mg/kg; 5, nanocrystalline silver 4 mg/kg; 6, nanocrystalline silver 0.4 mg/kg; 7, placebo; and 8, untreated. There is an absence of MMP-9 activity, but not MMP-2 activity, in the homogenates from 40 mg/kg and 4 mg/kg nanocrystalline silver and sulfasalazine treated rat and presence of MMP-9 and MMP-2 activities in the homogenates from rat treated with 0.4 mg/kg nanocrystalline silver, placebo, untreated and normal rat; 90% of the animals with oral treatment of 40 mg/kg of nanocrystalline silver reduced the MMP-9 activity to undetectable levels whereas only 40% of animals treated with 4 mg/kg and no animal in the 0.4 mg/kg treated group reduced the MMP-9 activity.

The results indicate that 100 mg/kg of sulfasalazine, 40 mg/kg of nanocrystalline silver and 4 mg/kg of nanocrystalline silver reduced MMP-9 activity, but not MMP-2 activity, measured by gelatin zymography. Nanocrystalline silver 0.4 mg/kg and placebo treatments were unable to inhibit the MMP-9 and MMP-2 activity.

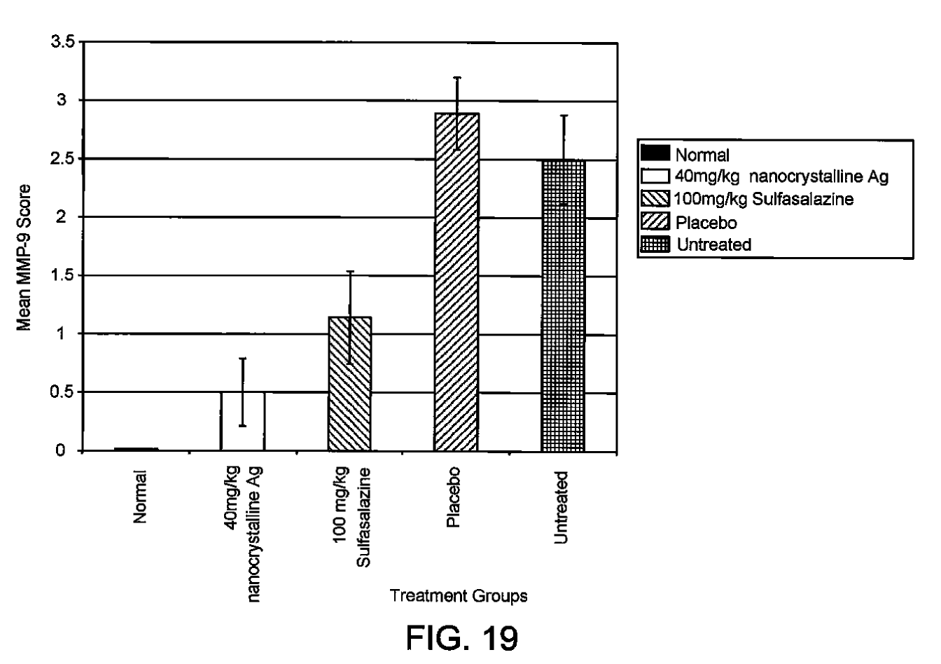

Expression of MMP-9 protein was also examined by immunohistochemical staining of the sections of colonic tissues with antibody to MMP-9. Nanocrystalline silver as well as sulfasalazine significantly reduced the expression of MMP-9 in the colonic tissues, compared to placebo and no treatment groups (FIG. 19).

Thus, intracolonic administration of DNBS induces reproducible ulcerative colitis in the rat model and this model has been used to study the efficacy of various anti-inflammatory compounds. The model was used to examine the anti-inflammatory effects of nanocrystalline silver as a nano-dispersion when administered intracolonically or orally. Quantitative macroscopic and microscopic observations in this study revealed that intracolonic treatment of nanocrystalline silver at the concentrations of 40 mg/kg, and 4 mg/kg significantly reduced colonic inflammation in the DNBS-induced ulcerative colitis in rat and was shown to be as effective as sulfasalazine, 100 mg/kg, whereas intra-colonic treatment of nanocrystalline silver at a concentration of 0.4 mg/kg was not effective.

Oral treatment of 40 mg/kg nanocrystalline silver also markedly improved colonic lesions and was as effective as oral treatment with 100 mg/kg of sulfasalazine. Nanocrystalline silver at concentrations of 4 mg/kg and 0.4 mg/kg were not effective orally. The increased potency of nanocrystalline silver with intracolonic treatment suggests that nanocrystalline silver is more effective when delivered locally to the target organ.

The effects seen with nanocrystalline silver were equivalent to those observed using sulfasalazine 100 mg/kg. Sulfasalazine is commonly used to treat IBD. Chemically, sulfasalazine is salicylazosulfapyridine (SASP) where sulfapyridine is linked to a salicylate radical by an azo bond. When taken orally, only a small proportion of the ingested dose is absorbed from the small intestine and the bulk of the sulfasalazine reaches the colon intact. The colonic bacteria split the azo bond with the liberation of sulfapyridine and 5-aminosalicylic acid (ASA), which is the active substance and exerts its anti-inflammatory activity locally.

As an approach to investigate the mechanism of the anti-inflammatory activity of nanocrystalline silver against colitis, the effect of oral nanocrystalline silver on the expression of inflammatory cytokines and modulation of MMP-9 was examined by measuring the protein expression of TNF-α, IL-12, IL-1 β and also studied the activity of MMP-9 by gelatin zymography. These cytokines and MMP-9 were up regulated in the colonic tissues of rat after induction of colitis. Nanocrystalline silver significantly suppressed the protein expression of TNF-α, IL-12 and IL-1β, whereas sulfasalazine significantly suppressed TNF-α, IL-1 β, but not IL-12. In addition, animals treated with nanocrystalline silver and sulfasalazine showed significant reduction of MMP-9 expression as well as gelatinase activity.

It has been reported that inflammatory cytokines including TNF-α, IL-12 and IL-1 and MMP-9 are up-regulated in experimental and human IBD. IL-1 and TNF-α share a multitude of proinflammatory properties and appear to be critical to the amplification of mucosal inflammation in IBD. Both cytokines are primarily secreted by monocytes and macrophages upon activation and up-regulate production of prostaglandin, proteases and other inflammatory and chemotactic cytokines.

The pathogenic role of IL-12 has been reported. IL-12 is produced by macrophages and lymphocytes after activation and stimulates NK cell activity and induces differentiation of CD4+ cells into Th1 cells.

MMP-9, also known as gelatinase B, is a zinc-dependent, calcium-requiring metalloproteinase, capable of degrading collagens as well as gelatins. MMP-9 has been implicated in the pathogenesis of inflammatory diseases. MMP-9 can be released by several cells in response to proinflammatory cytokines such as IL-1 and TNF-α and may be a key enzyme responsible for the accelerated breakdown of extra cellular matrix in colitis. It has been demonstrated that MMP-9 is abundantly expressed in patients with ulcerative colitis as well as experimental colitis. Furthermore, study has been shown that targeted deletion of MMP-9 attenuates experimental colitis suggesting the pathogenic role of this enzyme.

These results suggest that nanocrystalline silver, at a dose as low as 4 mg/kg when administered intracolonically, is effective to decrease signs of colitis in this model and is as effective as 100 mg/kg of sulfasalazine. In addition, it is suggested that the effect of nanocrystalline silver on suppression of inflammatory cytokines and MMP-9 may be responsible for its anti-inflammatory activity. This study suggests that nanocrystalline silver administered intracolonically or orally may have therapeutic potential for treatment of IBD.

### Example 26

### Anti-Inflammatory Activity of Nanocrystalline Silver-Containing Cream

The anti-inflammatory activity of 5% nanocrystalline silver-containing topical cream including 73.5% purified water, 1.5 percent polyoxyl 40 stearate, 1% glycerol monostearate, 5% stearic acid, 3% white petrolatum, 4% isopropyl myristate, 5% titanium dioxide, and 5% nanocrystalline silver, made as described in Example 10, and 2%, 1%, and 0% (placebo) nanocrystalline silver-containing topical cream formulations of Table 5B were evaluated using a guinea pig model of allergic contact dermatitis.

Methods: Allergic contact dermatitis was induced on the back of the guinea pigs using 5% dinitrochlorobenzene dissolved in acetone. Once the dermatitis was established, the test creams were applied topically once daily to the lesions for five days. Erythema and edema were evaluated daily (on a score of 0 to 4, from absent to very severe) and by histopathology of the skin biopsies after five days of treatment.

Results: This study demonstrates that placebo cream was unable to produce significant reduction of erythema and edema over five days of treatment. The groups treated with 5%, 2%, and 1% nanocrystalline-silver containing creams showed significant differences in edema score after one day and erythema score after two days of treatment, compared to placebo or no treatment groups (P<0.05). Skin biopsies, evaluated for degree of inflammatory response, mirrored the macroscopic observations. This study suggests that 5%, 2%, and 1% nanocrystalline silver-containing creams exhibit anti-inflammatory effects in this model.

Materials and Methods:

Two experiments were conducted. Where appropriate some results were combined.

Animals: Guinea pigs (female), weighing 250-300 gm, were purchased from Charles River Laboratories, Kingston, N.Y. The animals were housed in individual cages with 12-hour light and dark cycles, and allowed standard guinea pig chow and water ad libitum.

Chemicals: DNCB (1-chloro-2,4 dinitrochloro benzene lot #A0164370), and Acetone (lot # B0502510) were purchased from Acros Chemicals, Somerville, N.J.

Induction of allergic contact dermatitis in guinea pigs: allergic contact dermatitis was induced in guinea pigs as described in published literature. Guinea pigs were shaved and four 2-cm diameter areas were tattooed on their backs using a fine point permanent marker, two left and two right of the spinal midline. Animals were sensitized by applying 100 μl of 5% DNCB, dissolved in acetone, to the tattooed areas. Dermatitis was elicited 9 days later (day 10) with 50 μl of 5% DNCB applied to the same sites. Erythema and edema at each test site were graded on a scale of 0-4 as described below: 0: no visible erythema, no edema; 1: barely perceptible erythema, mild edema; 2: moderate numbers of pink-red erythema, moderate edema; 3: severe bright red erythema, severe edema; 4: very severe, marked erythema and crusting, extensive edema.

Treatment Groups: The following test compounds were used.

| Nanocrystalline silver formulation | Number of animals used |
|---|---|
| Placebo cream | 18 |
| 5% nanocrystalline silver cream | 8 |
| 2% nanocrystalline silver cream | 18 |
| 1% nanocrystalline silver cream | 18 |
| Control animals with ACD | 18 |

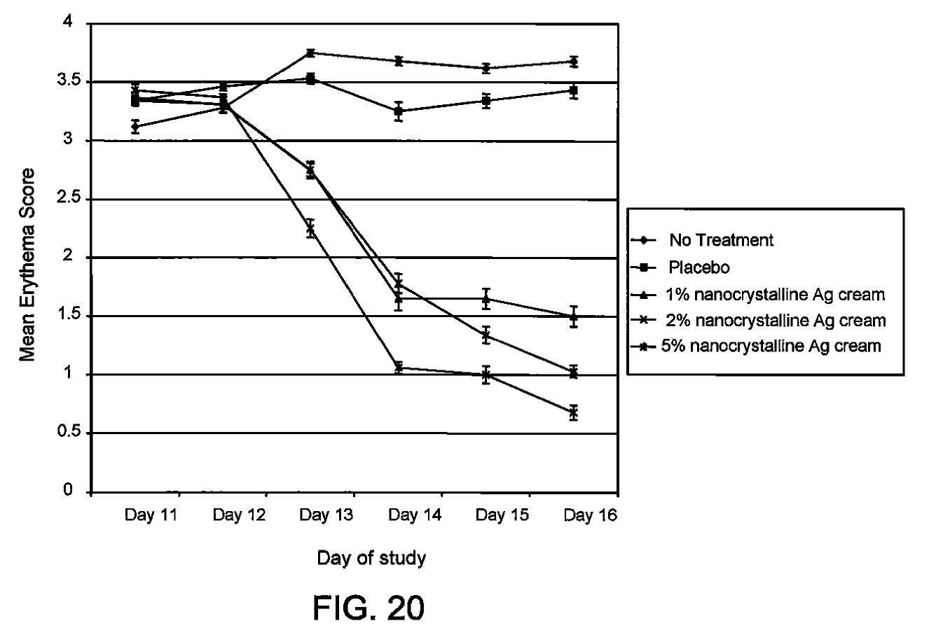

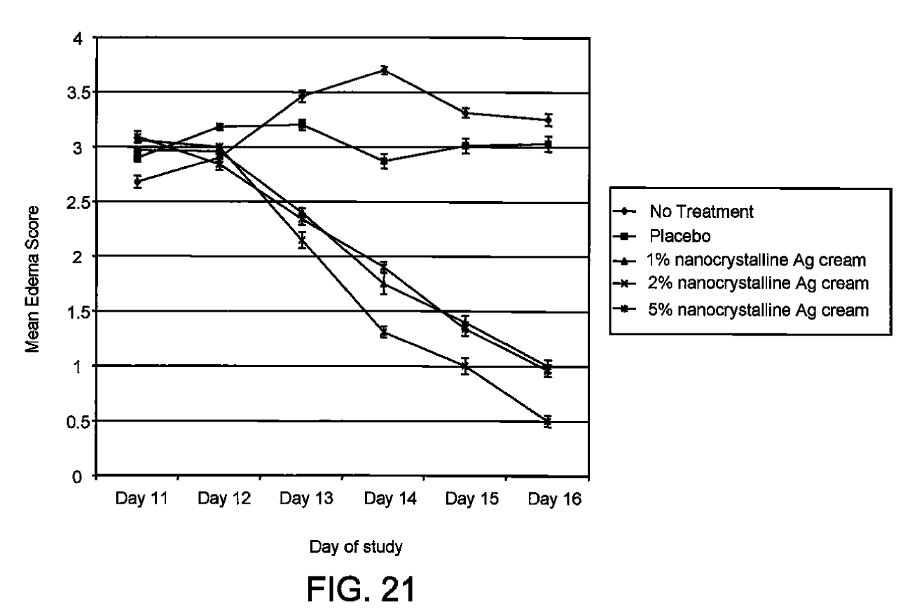

Treatment Procedure: One day after elicitation of dermatitis (day 11), the guinea pigs were evaluated for the presence of clinical development of erythema and edema, and divided arbitrarily into 5 groups of 8-10 animals. For all treatment groups, approximately 100 mg of test compounds was applied topically to each test site once daily for a total of five applications.
Histopathology: After five days of treatment (day 16), the animals were euthanized by intraperitoneal injection of pentobarbital (150 mg/kg). Using a sterilized punch (Uni Punch, Primer Medical Products, King of Prussia, Pa.), a four mm diameter full thickness punch biopsy was taken from one of the test sites arbitrarily selected from each animal. The biopsies were fixed in formalin solution and sent to Ameri Path (Boston, Mass.) for sectioning and staining with hematoxylin and eosin. The stained slides were blindly examined by a board certified pathologist (Department of Pathology, New England Medical Center, Tufts University, Boston, Mass.). The histological changes and the cellular infiltrates in the epidermis, superficial dermis, deep dermis and subcutis were graded as described below.
0: No changes or few scattered cells
1+: Mild changes, few inflammatory cells in several microscopic fields.
2+: Moderate changes, few to numerous inflammatory cells in all microscopic fields.
3+: Severe changes, numerous inflammatory cells in almost all microscopic fields,
4+: Very severe and extended changes, numerous inflammatory cells in masses, in all fields.
Statistical Method Scores for erythema and edema of each animal were obtained by averaging the four test sites. Scores for histopathological changes were obtained by taking the sum of the scores for epidermis, superficial dermis, deep dermis and subcutis. The statistical significance of differences for the erythema, edema and histopathological changes were assessed by Tukey-Kramer's multiple comparison tests following one-way analysis of variance (ANOVA). The data were calculated with the aid of INSTAT Graph Pad Software (San Diego, Calif.). The data were expressed as Mean±S.E. P-values less than 0.05 were considered statistically significant.
Results:
Two experiments were conducted. Where appropriate some results were combined.
Clinical evaluation: All guinea pigs developed dermatitis with clinical signs of erythema and edema after 24 hours of elicitation (second application of DNCB). The insult was moderate to very severe in almost all the test sites in all animals. The erythema and edema score ranged from 3+ to 4+ and 2+ to 4+, respectively. In the control group with no treatment, moderate to very severe erythema and edema persisted throughout the study (Tables 29, 30).
Effect of placebo cream on allergic contact dermatitis in guinea pigs: gross observation of the test sites demonstrated that the placebo cream did not produce significant reduction of erythema and edema compared to no treatment group. The mean scores of erythema and edema were 3.6±0.08 and 3.3±0.11 before treatment and after five days slightly decreased to 3.5±0.10 and 2.9±0.1, respectively (Tables 29 and 30, FIGS. 20 and 21).
Effect of 5% nanocrystalline silver-containing cream on allergic contact dermatitis in guinea pigs: The test sites treated with 5% nanocrystalline silver-containing cream showed a gradual decrease in erythema and edema (Tables 29 and 30, FIGS. 20 and 21). Significant differences in edema and erythema scores were observed within one and two days of treatment compared to the placebo (P<0.05). Significant differences in the erythema scores were observed during days 2 and 3 post treatment compared to 1% and 2% creams.
Effect of 2% nanocrystalline silver-containing cream on allergic contact dermatitis in guinea pigs: The test sites treated with 2% nanocrystalline silver-containing cream showed a gradual decrease in erythema and edema (Tables 29 and 30, FIGS. 20 and 21). Significant differences in edema and erythema scores were observed within one and two days of treatment compared to the placebo group (P<0.05).
Effect of 1% Nanocrystalline Silver-Containing Cream on Allergic Contact Dermatitis in Guinea Pigs:

The test sites treated with 1% nanocrystalline silver-containing cream showed a gradual decrease in erythema and edema (Tables 29 and 30, FIGS. 20 and 21). Significant differences in edema and erythema scores were observed within one and two days of treatment compared to the placebo group (P<0.05).

TABLE 29
Mean ± SE of erythema score (n = 8-18 animals) in the guinea pig model of
allergic contact dermatitis

| | | Day 12 - | Day 13 - | Day 14 - | Day 15 - | Day 16 - |
|---|---|---|---|---|---|---|
| | Day 11 - | 1 day | 2 days | 3 days | 4 days | 5 days |
| Treatment | day 0 of | after | after | after | after | after |
| groups | treatment | treatment | treatment | treatment | treatment | treatment |
| Placebo cream | 3.6 ± 0.08 | 3.8 ± 0.05 | 3.7 ± 0.07 | 3.5 ± 0.1 | 3.5 ± 0.09 | 3.5 ± 0.1 |
| 5% | 3.4 ± 0.05 | 3.3 ± 0.02 | 2.2 ± 0.07*† | 1.06 ± 0.04*† | 1.0 ± 0.07* | 0.68 ± 0.06* |
| nanocrystalline | | | | | | |
| Ag cream | | | | | | |
| 2.0% | 3.7 ± 0.09 | 3.5 ± 0.1 | 3.0 ± 0.1* | 1.8 ± 0.13* | 1.3 ± 0.16* | 1.2 ± 0.19* |
| nanocrystalline | | | | | | |
| Ag cream | | | | | | |
| 1.0% | 3.6 ± 0.07 | 3.4 ± 0.08 | 2.9 ± 0.12*† | 1.9 ± 0.18* | 1.4 ± 0.22* | 1.2 ± 0.19* |
| nanocrystalline | | | | | | |
| Ag cream | | | | | | |
| No Treatment | 3.5 ± 0.12 | 3.6 ± 0.09 | 3.7 ± 0.06 | 3.6 ± 0.07 | 3.4 ± 0.09 | 3.4 ± 012 |

*= P < 0.05 (Compared to placebo or No treatment groups)
†= P < 0.05 (Compared to 1 and 2% creams)

TABLE 30
Mean ± SE of edema score (n = 8-18 animals) in the guinea pig model of
allergic contact dermatitis.

| | | Day 12 - | Day 13 - | Day 14 - | Day15 - | Day16 - |
|---|---|---|---|---|---|---|
| | Day 11 - | 1 day | 2 days | 3 days | 4 days | 5 days |
| Treatment | day 0 | after | after | after | after | after |
| groups | treatment | treatment | treatment | treatment | treatment | treatment |
| Placebo cream | 3.3 ± 0.11 | 3.5 ± 0.09 | 3.5 ± 0.09 | 3.1 ± 0.12 | 3.3 ± 0.1 | 2.9 ± 0.1 |
| 5% | 3.06 ± 0.02 | 3.0 ± 0.0* | 2.1 ± 0.07* | 1.3 ± 0.04* | 1.0 ± 0.07* | 0.5 ± 0.05* |
| nanocrystalline | | | | | | |
| Ag cream | | | | | | |
| 2.0% | 3.3 ± 0.11 | 2.9 ± 0,06* | 2.6 ± 0.09* | 1.8 ± 0.08* | 1.3 ± 0.16* | 1.09 ± 0.16* |
| nanocrystalline | | | | | | |
| Ag cream | | | | | | |
| 1.0% | 3.3 ± 0.09 | 3.1 ± 0..06* | 2.5 ± 0.11* | 1.7 ± 0.17* | 1.3 ± 0.19* | 0.95 ± 0.15* |
| nanocrystalline | | | | | | |
| Ag cream | | | | | | |
| No Treatment | 3.08 ± 0.14 | 3.3 ± 0.12 | 3.5 ± 0.09 | 3.6 ± 0.1 | 3.1 ± 0.1 | 2.8 ± 0.1 |

*= P < 0.05 (Compared to placebo group)

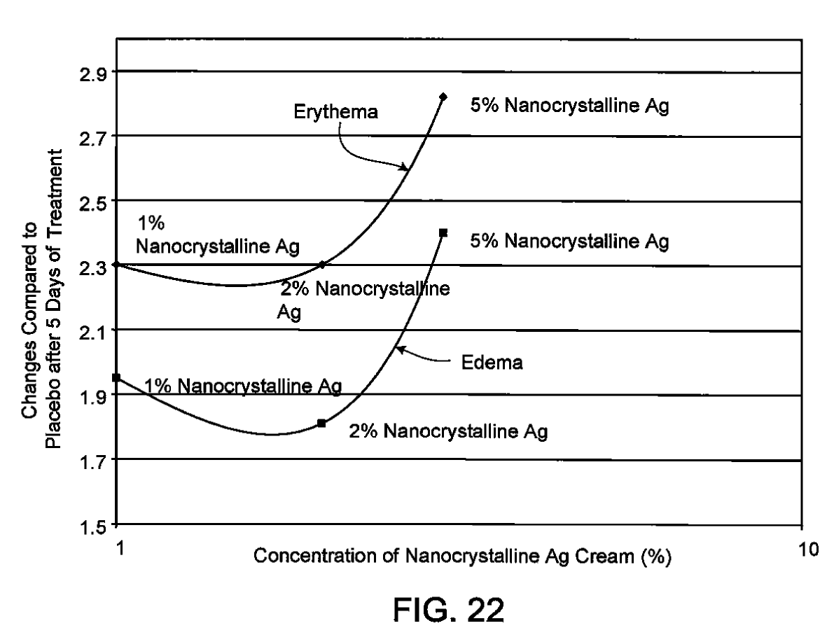

Concentration Response: When the difference in changes in erythema and edema was plotted against the different concentrations (5%, 2%, 1%) of nanocrystalline Ag creams, a dose response curve was obtained (FIG. 22).
Histopathological Observations: The histopathological changes and cellular infiltration in the epidermis, superficial dermis, deep dermis and subcutis were evaluated and graded as previously described. Generally the changes observed were epidermal vascularization, hyperkeratosis of the epidermis, ulcerated epidermis with large accumulations of necrotic neutrophils, necrotic and hemorrhagic blisters, vascular congestion, and intracellular edema. The cellular infiltrates consisted of mononuclear cells, neutrophils and eosinophils.

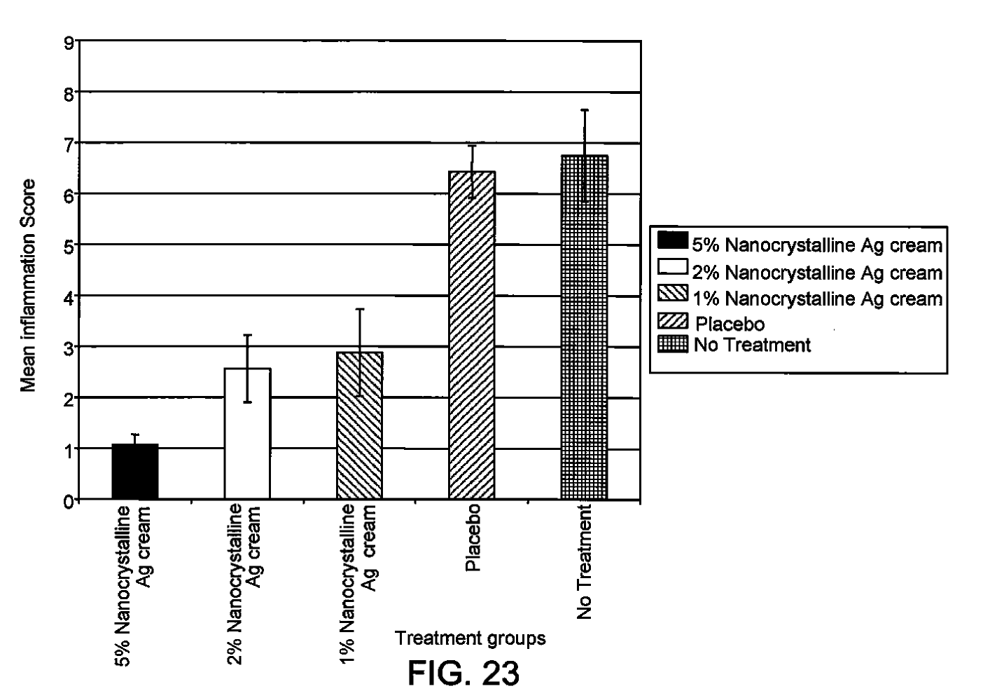

The sum of the individual scores from all the layers was calculated and the data plotted as the mean and standard error (Table 31, FIG. 23).

The histopathological analysis of the skin biopsies after five days of treatment demonstrated that creams containing 5%, 2%, and 1% nanocrystalline silver significantly reduced the inflammation compared to placebo and no treatment groups. No significant differences were observed among the groups treated with 5%, 2%, 1% creams. Placebo cream was unable to produce significant reduction of histopathological inflammation compared to the no treatment group.

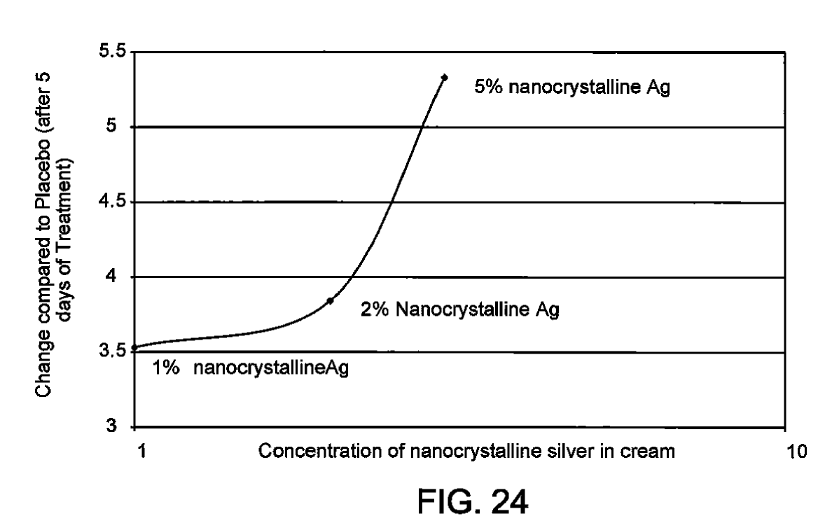

When the amount of inflammation was compared by subtracting the mean histopathological score among the nanocrystalline silver containing-cream treatments containing different concentrations of nanocrystalline silver and the placebo, a dose response was obtained. (FIG. 24).

TABLE 31
mean ± S.E of histopathological inflammation in the guinea
pig dermatitis model (n = 7-18). (The sum of the scores of
epidermis, superficial dermis, deep dermis and subcutis):

| | Mean ± S.E of |
|---|---|
| | histopathological inflammation |
| Treatment groups | score |
| Placebo cream | 6.4 ± 0.51 |
| 5% nanocrystalline Ag | 1.07 ± 0.2* |
| cream | |
| 2.0% nanocrystalline Ag | 2.56 ± 0.66* |
| cream | |
| 1.0% nanocrystalline Ag | 2.87 ± 0.85* |
| cream | |
| No Treatment | 6.75 ± 0.9 |

*= P < 0.05 compared to placebo and no treatment group.

The macroscopic and microscopic observations in this study revealed that the placebo cream did not suppress the inflammatory process in this guinea pig model of contact dermatitis. 5%, 2%, and 1% nanocrystalline silver-containing creams demonstrated the anti-inflammatory effect after two days of treatment. After five days of treatment, no statistically significant differences in erythema, edema or histopathological inflammation were observed among the groups treated with 5%, 2%, and 1% nanocrystalline silver-containing creams. The reduction in histopathological inflammation by 5%, 2% and 1% nanocrystalline silver-containing creams was statistically significant compared to placebo cream and no treatment groups, without significant differences among them.

In conclusion, this study suggests that nanocrystalline silver at the concentrations of 5%, 2%, and 1% nanocrystalline silver-containing creams are effective in reducing inflammation in this guinea pig model of allergic contact dermatitis.

### Example 27

### Effects of Silver Oxide in a Rat Model of DNBS-Induced Colitis

Methods: Colitis was induced in rats using DNBS as described in Example 25. Animals were treated intracolonically (250 μl of the test reagents) once a day for five days. Sonicated nanodispersions of Ag₂O or nanocrystalline silver were prepared as described in Example 15. Homogenized mixtures of Ag₂O were prepared by homogenizing a powder of Ag₂O using a ULTRA-TURRAX T 50 basic laboratory/pilot scale disperser/homogenizer (IKA Works).

| Group | | Actual % | Actual Dose | Number of |
|---|---|---|---|---|
| number | Description | of Ag | mg/kg | animals |
| 1 | 0.5% Ag₂O in | 0.41 | 3.4 | 9 |
| | in 1% PVA/Sonicated | | | |
| 2 | 0.25% Ag₂O in | 0.16 | 1 | 10 |
| | in 1% PVA/sonicated | | | |
| 3 | 0.5% Ag₂O in | 0.66 | 6.1 | 8 |
| | in 1% PVA/homogenized | | | |
| 4 | 0.25% Ag₂O in | 0.30 | 2.5 | 10 |
| | in 1% PVA/homogenized | | | |
| 5 | 0.5% nanocrystalline | 0.44 | 3.6 | 10 |
| | silver in 0.5% PVA | | | |
| | nanodispersion | | | |
| 6 | 0.25% nanocrystalline | 0.23 | 1.9 | 8 |
| | silver in 0.5% PVA | | | |
| | nanodispersion | | | |
| 7 | Sulfasalazine | | 100 | 10 |
| | 120 mg/ml | | | |
| 8 | Placebo/PVA | | | 8 |

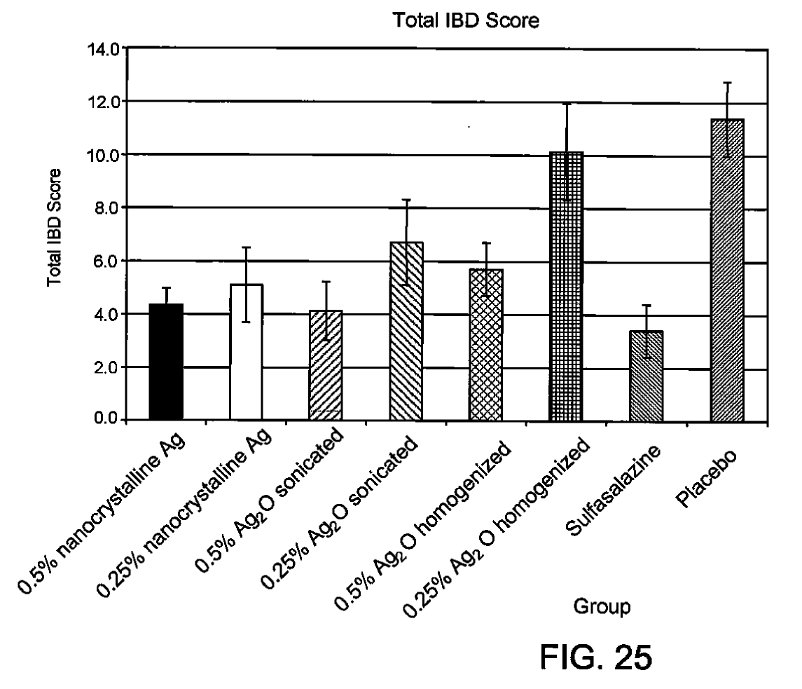

Referring to FIG. 25, nanocrystalline silver nanodispersion (0.5% and 0.25%) significantly reduced total IBD score compared to placebo group. Silver oxide (0.5% sonicated) but not 0.25% sonicated, significantly reduced the total IBD score compared to placebo. Silver oxide 0.5% and 0.25% (homogenized) were unable to show significant reduction of total IBD score compared to placebo. Sulfasalazine significantly reduced total IBD score compared to placebo. No significant differences in total IBD score were observed among 0.5%, 0.25% nanocrystalline silver nanodispersion, 0.5%, 0.25% sonicated silver oxide and 0.5%, 0.25% homogenized silver oxide. Sulfasalazine showed better reduction of total IBD score than 0.25% homogenized silver oxide.

This study shows that nanocrystalline silver nanodispersion (0.5% and 0.25%) was effective and as good as 100 mg/kg of sulfasalazine. Silver oxide 0.5% (sonicated) was effective in this model. Silver oxide (0.5%) homogenized was unable to show significant reduction of colonic inflammation. Nanocrystalline silver nanodispersion (0.5% and 0.25%) and 0.5% of sonicated silver oxide showed similar results.

### Example 28

### Concentration Response of Silver Oxide in a Rat Model of DNBS-Induced Colitis

Methods: Colitis was induced in rats using DNBS as described in Example 25. Animals were treated intracolonically (250 μl of the test reagents) once a day for five days. Sonicated nanodispersions of Ag₂O or nanocrystalline silver were prepared as described in Example 15.

Treatment Groups:

| Group | | Actual % | Actual Dose | | Number of |
|---|---|---|---|---|---|
| number | Description | of Ag | mg/kg | | animals |
| 1 | 1.5% Ag₂O in | 1.9 | 15.8 | | 10 |
| | in 1% PVA/sonicated | | | | |
| 2 | 0.5% Ag₂O in | 0.38 | 3.1 | | 10 |
| | in 1% PVA/sonicated | | | | |
| 3 | 0.15% Ag₂O in | 0.11 | 0.91 | | 9 |
| | in 1% PVA/sonicated | | | | |
| 4 | 0.05% Ag₂O in | 0.05 | 0.41 | | 10 |
| | in 1% PVA/sonicated | | | | |
| 5 | 0.5% nanocrystalline | 0.40 | 3.3 | | 10 |
| | silver in 0.5% PVA | | | | |
| 6 | Placebo | NA | NA | | 10 |
| 7 | Sulfasalazine | NA | 100 | mg/kg | 9 |
| | 120 mg/ml | | | | |

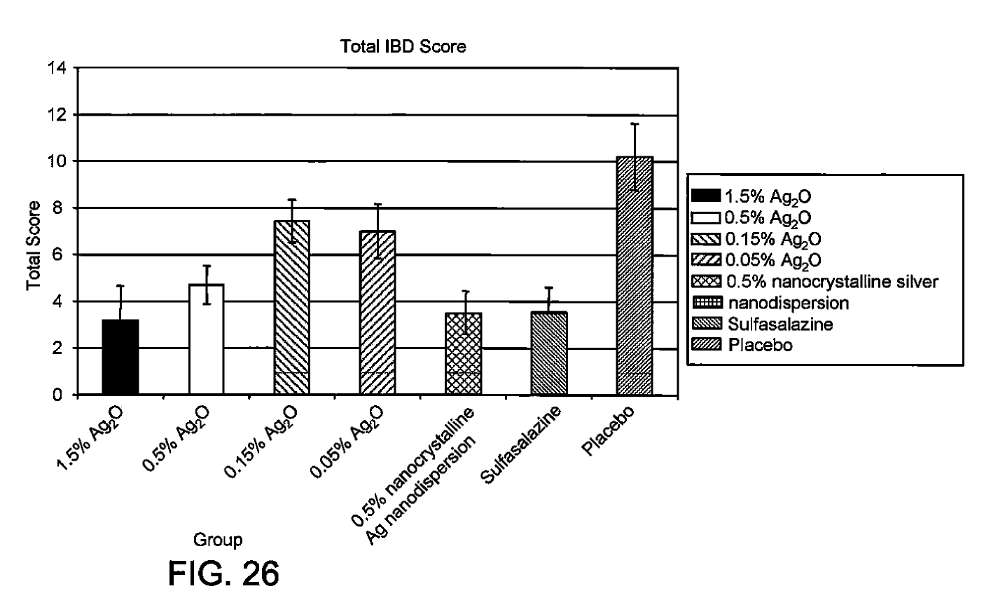

Referring to FIG. 26, nanocrystalline silver nanodispersion (0.5%) and sulfasalazine (100 mg/kg) significantly reduced total IBD score compared to placebo group. Silver oxide (Ag₂O) at the concentrations of 1.5% and 0.5% but not 0.15% and 0.05%, significantly reduced the total IBD score compared to placebo (without significant differences among them). No significant differences were observed among 0.5% nanocrystalline silver nanodispersion, 0.5% and 1.5% Ag₂O and sulfasalazine.

This study shows that nanocrystalline silver in nanodispersion (at a 0.5% concentration) was effective and as good as 100 mg/kg of sulfasalazine. Silver oxide nanodispersion at 1.5% and 0.5% were effective in this model. No statistical significant differences in the total IBD score were noticed among the groups treated with 1.5%, 0.5% silver oxide and 0.5% of nanocrystalline silver. No statistical significant differences in the total IBD score were noticed among the groups treated with 1.5%, 0.5% silver oxide and 100 mg/kg sulfasalazine. This study shows a concentration-response for Ag₂O in decrease of colitis in this model.

### Example 29

### Moisture Barrier Properties of Nanocrystalline Silver Containing Cream

Materials: 2% nanocrystalline silver cream (Table 5B), 1% nanocrystalline silver cream (Table 5B), placebo cream (Table 5B), 1% nanocrystalline silver in 8% White petrolatum cream, Eucerin Plus Intensive Repair Hand Cream, Eucerin Plus Intensive Repair Lotion, Eucerin Calming Cream, Whatman filter paper (lot H718144015014).

Procedure: 5 g of deionized water was weighed into each of 18 scintillation vials. Whatman filter paper was cut into 47 mm diameter circles, one for each vial. A Whatman membrane filter was fixed to the top of each vial using a rubber band. The edge of each vial (on top of the membrane and rubber band) was wrapped with parafilm. The weight of each vial with the membrane was recorded. A thin layer of cream weighing 50 mg was applied to the filter paper. Two vials were prepared for each cream and two vials without cream served as a control. Again the weight of the vials+membrane+cream (time zero) was recorded. The vials were placed into a 35° C./20% relative humidity stability chamber. The weight of the vials was recorded each day for 10 days to observe water loss. Table 32 shows the percent water loss of cream formulations over a 10 day period.

TABLE 32

| Total % Water | Day | | | | | | | |
|---|---|---|---|---|---|---|---|---|
| Lost | 0 | 1 | 2 | 3 | 7 | 8 | 9 | 10 |
| 2% nanocrystalline | 0 | 4.8063 | 9.9273 | 14.8576 | 34.7740 | 39.1907 | 44.8838 | 51.1343 |
| Ag cream | | | | | | | | |
| 1% nanocrystalline | 0 | 5.0278 | 10.0160 | 14.8174 | 34.4621 | 38.7419 | 44.2817 | 50.2579 |
| Ag cream | | | | | | | | |
| Placebo cream | 0 | 4.8411 | 9.2997 | 13.7762 | 31.8172 | 35.7769 | 40.7261 | 46.1362 |
| 1% nanocrystalline | 0 | 5.0004 | 10.4029 | 15.6717 | 37.4491 | 42.2051 | 48.3385 | 54.9848 |
| Ag, 8% white | | | | | | | | |
| petrolatum | | | | | | | | |
| White petrolatum | 0 | 4.4438 | 6.5031 | 9.8488 | 23.5375 | 26.5832 | 30.5012 | 34.9303 |
| Eucerin Hand | 0 | 3.9754 | 9.5850 | 14.0589 | 31.2674 | 34.9353 | 39.4192 | 44.2698 |
| Cream | | | | | | | | |
| Eucerin Lotion | 0 | 4.8304 | 22.2372 | 32.7081 | 74.4946 | 83.6129 | 96.7970 | 99.5339 |
| Eucerin Calming | 0 | 4.1107 | 16.0502 | 24.2520 | 57.5336 | 64.3708 | 73.6599 | 83.5881 |
| Cream | | | | | | | | |
| Control (no cream) | 0 | 2.9309 | 26.8962 | 39.4738 | 88.4426 | 98.3306 | 99.2519 | 99.2549 |

All references, such as patent applications, publications, and patents, referred to herein are incorporated by reference in their entirety.

Other embodiments are in the claims.

What is claimed is:

1. A method of treating inflammatory bowel disease, comprising:
contacting a gastrointestinal area of a subject with a therapeutically effective amount of a composition in the form of a nanodispersion,
wherein the composition comprises a pharmaceutically acceptable carrier, from 0.01 to five weight percent of a metal-containing material in the pharmaceutically acceptable carrier, and from 0.1 to ten percent by weight of a stabilizing agent in the pharmaceutically acceptable carrier.

2. The method of claim 1, wherein the metal-containing material comprises a nanocrystalline metal-containing material.

3. The method of claim 1, wherein the metal-containing material comprises an atomically disordered metal-containing material.

4. The method of claim 1, wherein the metal-containing material is selected from the group consisting of silver-containing compounds, platinum-containing compounds, palladium-containing compounds, and combinations thereof.

5. The method of claim 1, wherein the metal-containing material comprises nanocrystalline silver.

6. The method of claim 1, wherein the metal-containing material comprises silver oxide.

7. The method of claim 1, wherein the metal-containing material has a maximum dimension of 400 nanometers or less.

8. The method of claim 1, wherein the metal-containing material has a maximum dimension of 200 nanometers or less.

9. The method of claim 1, wherein the metal containing material has a dimension of at least ten nanometers.

10. The method of claim 1, wherein the stabilizing agent is selected from the group consisting of docusate sodium, sodium lauryl sulfate, cetrimide, PEG, povidone, propylene glycol, propylene glycol alginate, benzalkonium chloride, poloxamer, polyethylene alkyl ethers, sorbitan esters, xanthan gum, polyvinyl alcohol, lecithin, pectin, polysorbate, sorbitan, and combinations thereof.

11. The method of claim 1, wherein the composition comprises from 0.1 to two percent by weight of the stabilizing agent.

12. The method of claim 1, wherein prior to incorporation into the composition the metal-containing material has a surface charge, and when incorporated into the composition the stabilizing agent attenuates the surface charge.

13. The method of claim 1, wherein the composition further comprises a buffered solution in the pharmaceutically acceptable carrier.

14. The method of claim 13, wherein the buffered solution is selected from the group consisting of lactate buffer, EDTA buffer, citrate buffer, and gluconate buffer.

15. The method of claim 13, wherein, prior to incorporation in the composition, the buffer solution has a pH of from 3 to 9.

16. The method of claim 1, wherein the composition is in a form selected from the group consisting of an emulsion, an enema, a foam, a wash, a drop, and a spray.

17. The method of claim 1, wherein the therapeutically effective amount is at least 0.4 mg of metal-containing material per kg of the subject.

18. The method of claim 1, wherein the therapeutically effective amount is at least 4 mg of metal-containing material per kg of the subject.

19. The method of claim 1, wherein the therapeutically effective amount is at least 40 mg of metal-containing material kg of the subject.

20. The method of claim 1, wherein the composition comprises at most 600 mg of metal-containing material per dose of the composition.

21. The method of claim 1, wherein the composition comprises at least 20 mg of metal-containing material per dose of the composition.

22. The method of claim 1, wherein contacting the area comprises a dosage of the composition at least one dose per 24 hours.

23. The method of claim 1, wherein contacting the area comprises a dosage of the composition at least one dose per 12 hours.

24. The method of claim 1, wherein contacting the area comprises a dosage of the composition at least one dose per six hours.

25. The method of claim 1, wherein contacting the area comprises a dosage of the composition at least one dose per three hours.

26. A method of treating inflammatory bowel disease, comprising:
contacting a gastrointestinal area of a subject with a composition at a dosage of from one dose per three hours to once per 24 hours,
wherein the composition comprises a pharmaceutically acceptable carrier, a metal-containing material, and a stabilizing agent,
the composition comprises from 20 to 600 mg of the metal-containing material per dose, and
the composition is a nanodispersion.

27. A method of treating inflammatory bowel disease, comprising:
contacting a gastrointestinal area with a therapeutically effective amount of a composition,
wherein the composition comprises a freeze-dried powder including a metal-containing material, a stabilizing agent, and a bulking agent.

28. The method of claim 27, wherein the freeze-dried powder is in the form of a suppository.

29. The method of claim 27, wherein the freeze-dried powder is in the form selected from the group consisting of a tablet, a pill, and a capsule.

30. The method of claim 27, wherein the metal-containing material comprises silver.

31. The method of claim 30, wherein the bulking agent is selected from the group consisting of mannitol, glycine, gelatin, dextran, glucose, sucrose, lactose, and combinations thereof.

32. The method of claim 27, wherein the freeze-dried powder further comprises a cryoprotectant.

33. The method of claim 32, wherein the cryoprotectant is selected from the group consisting of glycine, glucose, fructose, sucrose, lactose, and combinations thereof.

34. The method of claim 27, wherein the stabilizing agent is selected from the group consisting of docusate sodium, sodium lauryl sulfate, cetrimide, PEG, povidone, propylene glycol, propylene glycol alginate, benzalkonium chloride, poloxamer, polyethylene alkyl ethers, sorbitan esters, xanthan gum, polyvinyl alcohol, lecithin, pectin, polysorbate, sorbitan, and combinations thereof.

35. The method of claim 27, wherein the metal-containing material comprises a nanocrystalline metal-containing material.

36. The method of claim 27, wherein the metal-containing material comprises an atomically disordered metal-containing material.

37. The method of claim 27, wherein the metal-containing material is selected from the group consisting of silver-containing compounds, platinum-containing compounds, palladium-containing compounds, and combinations thereof.

38. The method of claim 27, wherein the metal-containing material comprises nanocrystalline silver.

39. The method of claim 27, wherein the metal-containing material comprises silver oxide.

40. A method of treating inflammatory bowel disease, comprising:
contacting a gastrointestinal area of a subject with a composition at a dosage of from one dose per three hours to once per 24 hours,
wherein the composition comprises a pharmaceutically acceptable carrier, a metal-containing material, and a stabilizing agent,
the composition comprises at least 0.4 mg of metal-containing material per kilogram of subject per dose, and
the composition is a nanodispersion.

41. A method of treating inflammatory bowel disease, comprising:
reconstituting a freeze-dried powder of a metal-containing material to form a composition comprising the metal-containing material; and
contacting a gastrointestinal area of a subject with a therapeutically effective amount of the composition.

42. The method of claim 41, wherein the composition comprises a pharmaceutically acceptable carrier, from 0.01 to five weight percent of a metal-containing material in the pharmaceutically acceptable carrier, and from 0.1 to ten percent by weight of a stabilizing agent in the pharmaceutically acceptable carrier.
